(19) United States
(12) Patent Application Publication
(10) Pub. No.: US 2006/0258652 A1
(43) Pub. Date: Nov. 16, 2006

(54) Beta-blockers having antioxidant and no-donor activity

(76) Inventors: Abdullah I Haj-Yehia, NEVE SHALOM (IL); Mohamed Amin Khan, Morgan Hill, CA (US)

Correspondence Address: MORRISON & FOERSTER LLP, 755 PAGE MILL RD, PALO ALTO, CA, 94304-1018, US

(21) Appl. No.: 10/535,620

(22) PCT Filed: Nov. 21, 2003
(86) PCT No.: PCT/IL03/00986

§ 371 Date: Jan. 4, 2006

Related U.S. Application Data
(60) Provisional application No. 60/428,502, filed on Nov. 22, 2002.
(60) Provisional application No. 60/428,733, filed on Nov. 22, 2002.

(51) Int. Cl. A61K 31/5383; A61K 31/452; A61K 31/433; A61K 31/381; A61K 31/4025; A61K 31/385; A61K 31/407

(52) U.S. Cl. 514/230.5; 514/362; 514/411; 514/416; 514/439; 514/440; 514/422; 514/320

(57) ABSTRACT

Multifunctional beta-antagonist compounds are provided, that combine β-adrenergic receptor blocking activity with capability to scavenge superoxide and other reactive oxygen species, and that may further function as nitric oxide donors. The compounds are useful for preventing or treating disorders in which the treatment with β-antagonists is indicated, or which are associated with oxidative stress.

### TECHNICAL FIELD

The present invention relates to multifunctional β-adrenergic receptor antagonist compounds (β-blockers) that are capable, in addition to their β-blocking activity, of acting as scavengers of superoxide or other reactive oxygen species, and optionally also as nitric oxide donors. The invention further relates to methods of using such compounds in the treatment of various pathological conditions.

### BACKGROUND OF THE INVENTION

NO (nitric oxide) is formed from the amino acid L-arginine by several forms of NO synthases, and plays a role in a number of physiological functions, including the relaxation of airway smooth muscle. NO formed in endothelial cells in response to chemical agonists and to physical stimuli plays a key role in the regulation of vascular tone, in the platelet aggregation and adhesion, as well as in modulating smooth muscle proliferation [Haj-Yehia et al: Drug. Development Res. 50 (2000) 528-536]. NO overproduction has also been associated with numerous disease states (WO 99/66918).

Publications disclosing nitric oxide donor compounds or compounds which promote the synthesis of nitric oxide include WO 98/42661, WO 99/37616, WO 00/31060, WO 97/34871, WO 00/35434, WO 99/62509, WO 97/25984, WO 00/67754, WO 9961018, WO 99/61430, WO 97/31654, WO 96/32946, WO 00/53191, U.S. Pat. Nos. 6,248,895 and 6,232,331, and Wolf et al: J. Neurosurg. 89 (1998) 279-288]. Publications disclosing nitric oxide scavenger compounds include WO 98/55453.

The endothelium, in addition to producing NO, also produces superoxide (SO) anion and other reactive oxygen species (ROS) under physiological conditions. Despite SO being a reducing agent that is itself incapable of initiating oxidative reactions, SO is considered to be the most important source of oxidative stress. Compounds for the removal of SO are described in the art, including WO 96/39409 and U.K. Pat. App. No. 2349385A.

Many disease states, including diabetes mellitus and various cardiovascular diseases, are associated with oxidative stress and endothelial dysfunction. Nitroglycerin (GTN) has been used for the treatment of various types of myocardial ischemia. Because of its pathogenic nature (chronicity with acute exacerbation), prophylactic and acute treatments are necessary to prevent complications with potentially fatal outcomes (>25% death for acute MI). However, the phenomenon of tolerance to the anti-anginal effects of GTN and to all other existing organic nitrates is of a special clinical significance. In particular, early development of tolerance to the drug is by far the most serious drawback of nitrate therapy.

A number of cardiovascular conditions have been recognized, (e.g., angina, hypertension, arrhythmias, congestive heart failure) and a number of other conditions (e.g., migraine, tachycardia (e.g., sinus, pheochromocytoma, thyrotoxicosis), tension, anxiety, and the symptoms of hyperthyroidism) have been recognized, many of which have overlapping and interacting etiologies.

Various compounds and treatments for cardiovascular conditions are disclosed in the art, for example, in U.S. Pat. Nos. 6,444,702, 6,417,207, 6,255,296, 6,051,571, 6,440,961, 6,429,219, 6,423,724, and 6,248,895.

Similarly, compounds and treatments for migraines are disclosed in the art, for example, U.S. Pat. Nos. 6,458,840, 6,458,830, 6,444,702, 6,376,550, 6,414,505, 6,403,627, 6,355,689, 6,331,553, 6,265,441, 6,423,724, and 6,455,549.

Various compounds and treatments for sinus tachycardia are disclosed in the art, for example, U.S. Pat. No. 6,100,297.

Compounds and treatments for hypertension are disclosed in the art, for example, U.S. Pat. Nos. 6,440,961, 6,429,219, 6,423,724, 6,214,817, and 6,455,542.

Various compounds and treatments for the symptoms of hyperthyroidism are also disclosed in the art, for example, U.S. Pat. Nos. 6,110,959, 6,121,309, and 6,437,165.

β-Adrenergic receptor antagonist agents remain the cornerstone for therapy of all stages of ischemic heart disease. They constitute the standard therapy for effort angina, mixed effort and rest angina, and unstable angina They decrease mortality in acute-phase myocardial infarction and in the post-infarct period. In addition to their primary role in the treatment of ischemic heart disease, β-blockers retain their leading position among basic therapies for other cardiovascular conditions including hypertension, arrhythmias, cardiomyopathy, and congestive heart failure.

β-antagonists also possess other properties that turn them useful for the treatment of non-cardiovascular conditions. These include central indications (e.g., anxiety, essential tremor, migraine prophylaxis, alcohol withdrawal), endocrine conditions (e.g., thyrotoxicosis), gastrointestinal (e.g., esophageal varices), and ocular conditions (e.g., topical use for glaucoma, reduction of intraocular fluid).

Adverse effects of β-antagonists are generally related to the pharmacological consequences of blockade of the β receptors. These adverse effects may include induced congestive hear failure, induced or exacerbated heart failure in individuals with compensated heart failure, acute myocardial infarction or cardiomegaly. Additionally, β-antagonists may cause the blockage of β₂-receptors in bronchial smooth muscle, thereby increasing airway resistance. Treatment with β-antagonists may also result in adverse effects linked to the central nervous system, such as fatigue, sleep disturbances, memory loss and depression, while the use of β-antagonists may also mask the symptoms of hypoglycemia in diabetic individuals. Thus, β-antagonists should be used with caution for individuals with diabetes. See also Cotran et al., “Robbins Pathologic Basis of Disease” 4th Ed. 1989, W.B. Saunders Co., Philadelphia, Pa.

There is a need for improved drugs for the treatment of cardiovascular conditions, (e.g., angina, hypertension, arrhythmias, congestive heart failure) and a number of other conditions (e.g., migraine, glaucoma, sinus tachycardia, tension, and the symptoms of hyperthyroidism, etc.).

### BRIEF SUMMARY OF THE INVENTION

This invention relates to multifunctional beta-antagonist compounds possessing, beside β-antagonist activity, also antioxidant activity that enables scavenging reactive oxygen species (ROS), and optionally also nitric oxide (NO) donating capability.

This invention is also directed to a method for treating and preventing a disorder selected from the group consisting of disorders in which treatment with a β-antagonist is indicated, disorders associated with oxidative stress and free radical injury, and disorders in which treatment with a smooth muscle relaxant is indicated, comprising administering said multifunctional β-blocker compounds. The use of said compounds in the preparation of a medicament is further provided. Preferred disorders to be treated and prevented according to this invention comprise cardiovascular, pulmonary, neurological, hormonal, gastrointestinal, and ocular disorders, examples being ischemia, ischemia-reperfusion tissue injury, acute and chronic inflammatory conditions, angina, atherosclerosis, impotence, hypertension, pulmonary hypertension, systemic hypertension, obesity or pregnancy-induced hypertension, palpitations, arrhythmias, cardiomyopathy, congestive heart failure, hyperthyroidism, anxiety, tremor, migraine, alcohol withdrawal, tachycardia, thyrotoxicosis, pheochromocytoma, esophageal varices, glaucoma, conditions associated with excess intraocular fluid, diabetes mellitus, and carcinogenesis. Said multifunctional compounds may be also used for treating or preventing an adverse effect caused by other β-antagonists.

This invention further provides a multifunctional β-adrenergic receptor antagonist compound comprising i) a β-blocker component, ii) at least one ROS-scavenger component, and optionally iii) at least one NO-donor component. Said β-blocker component may comprise compounds used in medicine as β-adrenergic blockers or derivatives thereof, as well as other compounds exhibiting affinity for β-receptors. Said multifunctional antagonist comprises a ROS-scavenger component that may be an antioxidant reacting with ROS, such as superoxide, hydroxyl radicals, peroxynitrite, and hypochlorite. A preferred ROS-scavenger component may be, for example, selected from a substituted N-oxide free radical, a substituted or unsubstituted lipoic acid moiety; examples of said NO-donor component comprise —ONO₂, —ONO, —SNO, and —NONOate. Said β-blocker component may be selected from β-antagonists used in medicine, or their derivative. Examples of such known β-antagonists comprise Carteolol, Oxprenolol, Nadolol, Propranolol, Metoprolol, Metipranol, Pindolol, Betaxolol, Atenolol, Esmololol, Levobunolol, Labetalol, and Tomolol.

In a preferred embodiment of this invention, a multifunctional antagonist may have Formula I 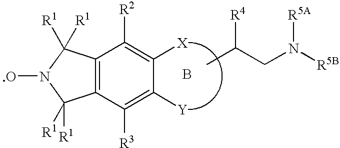
wherein R¹ may be for example alkyl, R¹ and R³ may be for example hydrogen or CH₂OH, R⁴ may be for example OH, R^{5A} may be for example hydrogen, R^{5B} may be for example N-oxide free radical, X may be for example O, Y may be for example (CH₂)₂, B may be a 5 membered ring.

In another preferred embodiment of this invention, a multifunctional antagonist may have formula Formula II

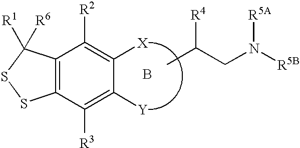

wherein R¹ and R⁶ may be for example alkyl, R² and R³ may be for example hydrogen, R⁴ may be for example OH, R^{5A} may be for example hydrogen, R^{5B} may be for example N-oxide free radical, X may be for example O, Y may be for example (CH₂)₂, and B may be a 5 membered ring R^{5A}.

In still another preferred embodiment of this invention, a multifunctional antagonist may have Formula IA

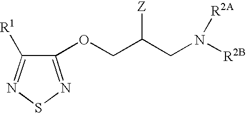

wherein R¹ may be for example a ROS scavenger group, Z may be for example nitrato, R^{2A} and R^{2B} may be for example hydrogen and alkyl.

In a preferred embodiment of this invention, multifunctional antagonists, comprising both new antagonists as discosed here and antagonists used in the art, have formula III

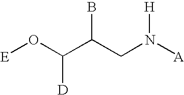

wherein A is C₁-C₄ alkyl or ROS-scavenger group;

B is selected from OH, O—NO₂ and SH;

D is H, or D is (CH₂)₂ that is connected to E forming together with the neighboring atoms a 5-6 membered ring consisting of carbon atoms and one oxygen atom; and

E is phenyl condensed with optionally substituted phenyl or optionally substituted 5-6 membered heterocycle containing one of —N—, —O—, and —S—S—; or

E is thiadiazolyl substituted with morpholinyl or pyrrolidinyl-N-oxide, said morpholinyl being optionally substituted with one of OH, NO-donor group, and ROS-scavenger group, and said pyrrolidinyl-N-oxide group being bound to said thiadiazolyl.

A pharmaceutical composition is further provided, comprising at least one multifunctional beta-antagonist compound, or a solvate, optical isomer, and salt thereof, and at least one pharmaceutically acceptable excipient, diluent, propellant, etc.

### BRIEF DESCRIPTION OF THE DRAWINGS

FIGS. 1 to 3 show exemplary beta-antagonist compounds which may be modified to form multifunctional beta-antagonist compounds.

FIG. 4 shows exemplary multifunctional beta-antagonist compounds 14, 15, 20 and 21.

FIG. 5 shows multifunctional beta-antagonist compounds 22-25.

FIG. 6 shows multifunctional beta-antagonist compounds 26-30.

FIG. 7 shows multifunctional beta-antagonist compounds 31-35.

FIG. 8 shows multifunctional beta-antagonist compounds 36-40.

FIG. 9 shows multifunctional beta-antagonist compounds 41-45.

FIG. 10 shows multifunctional beta-antagonist compounds 46-50.

FIG. 11 shows multifunctional beta-antagonist compounds 51-55

FIG. 12 shows multifunctional beta-antagonist compounds 56-60.

FIG. 13 shows multifunctional beta-antagonist compounds 61-65.

FIG. 14 shows multifunctional beta-antagonist compounds 66-70.

FIG. 15 shows multifunctional beta-antagonist compounds 71-75.

FIG. 16 shows multifunctional beta-antagonist compounds 1′, 2′, and 17′-20′.

FIG. 17 shows multifunctional beta-antagonist compounds 21′-24′.

FIG. 18 shows multifunctional beta-antagonist compounds 7′-10′.

FIG. 19 shows multifunctional beta-antagonist compounds 11′-13′.

FIG. 20 shows multifunctional beta-antagonist compounds 14′-16′.

### DETAILED DESCRIPTION OF THE INVENTION

Provided are multifunctional β-antagonist compounds, and compositions comprising the multifunctional β-antagonist compounds, which may be used in methods of treating ocular, cardiovascular, pulmonary, neurological, endocrine, gastrointestinal, and other conditions as described herein. The multifunctional β-antagonist compounds, compositions comprising the multifunctional β-antagonist compounds and methods for use of such multifunctional beta-antagonist compounds described herein are also directed to avoiding adverse effects, development of tolerance (e.g., desensitization) or hypersensitivity on repeated administration.

The multifunctional beta-antagonist compound of this invention includes a beta-antagonist component, a ROS scavenger component (e.g., SOD mimic) and, optionally, a nitric oxide donor component. Thus, in one embodiment, a beta-antagonist is provided in modified form and includes a SOD mimic component and a nitric oxide donor component capable of releasing NO in a charged or neutral form. The beta-antagonist component may be linked to at least one ROS scavenger component, and optionally to at least one nitric oxide donor component. The anticipated superior beneficial therapeutic effects of multifunctional beta-antagonist compounds may be attributed to their simultaneous multi-mechanistic actions as β-receptor blockers (see diverse pharmacological actions described herein), SOD-mimics and/or ROS scavengers (antioxidant and anti-inflammatory that provide additional cellular protection), and, optionally, as NO-donors (vasodilator, antioxidant, anti-proliferative, cellular protectant). These properties are vital for adequate prevention and/or treatment of cardiovascular conditions involving ischemia, angina, hypertension, palpitations, arrhythmias (e.g., supraventricular, ventricular), cardiomyopathy, and congestive heart failure, as well as other conditions for which the use β-receptor blocking agents is indicated.

The multifunctional beta-antagonist compounds and functionalized beta-antagonist compounds described herein may also be used in the manufacture of a medicament for treating conditions in which treatment with a beta-antagonist is indicated, as described herein.

In particular, described herein are nitrosated or nitrosylated β-receptor blocking agents possessing SOD-mimic and/or ROS scavenger components which can optionally be substituted with at least one ONO, SNO, or ONO₂ moiety, or a compound that donates, transfers, or releases nitric oxide in either a neutral or a charged form.

The multifunctional beta-antagonist compounds offer a new strategy for the treatment of various diseases that can alter not only the clinical symptoms of the disease, but also its pathogenesis, natural course and outcome.

The multifunctional β-antagonist compounds, and compositions comprising the multifunctional β-antagonist compounds, may be used in methods of treating ocular conditions (e.g., glaucoma) as well as cardiovascular conditions and other conditions.

The multifunctional β-antagonist compounds, and compositions comprising the multifunctional β-antagonist compounds described herein not only provide a source of nitric oxide, which acts in the regulation of cardiopulmonary function, but, in acting as an antioxidant scavenger of superoxide anion and other reactive oxygen species, give rise to a direct benefit derived from removal of injurious superoxide anion and other reactive oxygen species, and to a benefit in protecting both ambient and endogenous, as well as liberated exogenous, NO from inactivation by superoxide anion and other reactive oxygen species. These properties of the multifunctional beta-antagonist compounds create a beta-receptor (e.g., β₂) antagonist with superior characteristics as compared with non-functionalized beta-antagonists (e.g., more potent vasodilators, ability to administer lower dosages, reduced toxicity). These factors prevent the development of an interference from tolerance, and reduce toxicity levels (from the donated NO or the parent molecule) compared to non-functionalized beta-antagonist compounds or beta-antagonist functionalized with NO-donor alone. Additionally, oxidative stress plays an important role in the pathogenesis, progression and severity of the diseases mentioned above (cardiovascular, ocular etc) by acting in concert with other pathogenic mediators. Therefore, multifunctional β-antagonist compounds, have the advantage of beneficially modulating multiple pathways that determine the pathogenesis, progression and severity of the disease at the site of required drug action.

When describing the multifunctional beta-antagonist compounds comprising one or more NO-donor component and one or more ROS scavenger component, pharmaceutical compositions comprising the multifunctional beta-antagonist compounds and methods making or using the multifunctional beta-antagonist compounds, the following terms have the following meanings unless otherwise specified.

As used herein, the term “multifunctional β-antagonist compound” refers to a compound containing a β-antagonist component, and additionally at least one antioxidant component, such as an ROS scavenger component, and optionally at least one NO donor component. The components may be linked, for example directly, indirectly and/or via a sharing of atoms, as described herein. The use of the term “multifunctional β-antagonist compound” is not intended to necessarily require that the compound was formed by chemical modification of a β-antagonist, since the synthesis would not necessarily involve a starting material that was a β-antagonist that is further modified, and other routes of synthesis are contemplated. Rather, a “multifunctional β-antagonist compound” is meant to be a molecule that not only includes a β-antagonist component with β-antagonist activity, but also the additional antioxidant functionality (such as ROS scavenger), and possibly a NO-donor components. Thus, in one embodiment, multifunctional β-antagonist compounds are provided that are β-antagonists in a modified form wherein they include an a ROS scavenger component and NO donor component. The terms “multifunctional β-antagonist compound”, “multifunctional beta-antagonist compound”, “multifunctional beta-receptor blocking compound” and “multifunctional beta-blocker compound”, may be used interchangeably herein.

NO Donors

Groups that can act as nitric oxide donors are capable of acting as a source of nitric oxide (NO). The nitric oxide donor component is, for example, an —NO₂—ONO, —SNO or —NONOate group. In particular embodiments the NO donor component is —NO₂ or —SNO. The NO donor component, for example, donates, transfers, or releases nitric oxide in either a neutral or a charged form. The nitric oxide donor component may comprise any group capable of acting as a source of nitric oxide (NO) in a charged or uncharged form, including nitrosonium (NO+), nitroxyl (NO−) or nitric oxide (NO.).

Reactive Oxygen Species Scavengers

The multifunctional β-antagonist compound includes a chemical moiety that can function as an antioxidant component, preferably without affecting the stability and action of an eventually present NO-donor component. The antioxidant component can be a reactive oxygen species (ROS) scavenger. As used herein, the term “reactive oxygen species (ROS) scavenger component” refers to a moiety capable of acting as a scavenger of, or reacting with, superoxide (O₂⁻) or other reactive oxygen species (ROS) including hydroxyl radicals, peroxynitrite, hypochlorous acid and hydrogen peroxide. An antioxidant that preferentially scavenges, or reacts with, superoxide is termed a “superoxide dismutase mimic” (“SOD-mimic”), superoxide scavenger, or “superoxide dismutase mimetic” (“SOD-mimetic). The reactive oxygen species superoxide (O₂⁻), hydroxyl radicals, peroxynitrite, hypochlorous acid and hydrogen peroxide are considered biologically undesirable, while nitric oxide, as described above, may be biologically beneficial. Thus, the antioxidant or ROS scavenger component preferably does not react with, or scavenge, nitric oxide.

The multifunctional β-antagonist compounds described herein may include one or more antioxidant or ROS scavenger components. In some embodiments, the reactive oxygen species scavenger component is a nitroxide free radical (NO.) group. In certain embodiments the compounds as described herein may comprise more than one ROS scavenger component, for example at least one, at least two, at least three or at least four ROS scavenger components.

As used herein, the ROS scavenger component itself is not intended to be a group capable of donating nitric oxide (NO). Further, the ROS scavenger component is provided in addition to the β-antagonist component of the multifunctional β-antagonist compound.

The antioxidant component, such as an ROS scavenger component, may be for example an alkenyl group; aryl group; substituted aryl group, where the aryl group is substituted with, for example, —OH, —NH₂, —NHCHO or a NO donor group; sulfhydryl (in a protected form) or dithiol in oxidized or reduced form; or a group that is, or is capable of being converted in vivo into, a sulfhydryl in its oxidized or reduced form.

As used herein, the term “alkyl” includes branched or unbranched hydrocarbon chains, having for example, about 1 to about 18 carbons, such as methyl, ethyl, n-propyl, iso-propyl, n-butyl, sec-butyl, iso-butyl, tert-butyl, octa-decyl and 2-methylpentyl. Alkyl may also include cyclic alkyl groups, for example, including about 5-8 carbons, such as cyclopentyl, cyclohexyl, cycloheptyl, or cyclooctyl. The term “lower alkyl” refers to an alkyl group having from 1 to 6 carbon atoms.

“Substituted alkyl” refers to alkyl substituted with one or more functional groups such as hydroxyl, bromo, fluoro, chloro, iodo, mercapto or thio, cyano, alkylthio, aryl, carboxyl, carbalkoyl, alkenyl, nitro, amino, amido, and an NO donor group. A cyclic alkyl group may be substituted with a straight or branched alkyl group. Substituted alkyl further refers to acyl, acylamino, acyloxy, alkoxy, substituted alkoxy, alkoxycarbonyl, alkoxycarbonylamino, substituted amino, aminocarbonyl, aminocarbonylamino, aminocarbonyloxy, aryloxy, azido, keto, thioalkoxy, substituted thioalkoxy, thioaryloxy, thioketo, alkyl-S(O)—, aryl-S(O)—, alkyl-(O)₂— or aryl-S(O)₂—. Substituted alkyl may further refer to an alkyl group having from 1 to 5 substituents, or from 1 to 3 substitutents.

The term “aryl” includes a chain of carbon atoms which form at least one aromatic ring having, for example, between about 6-14 carbon atoms, such as phenyl, naphthyl, anthracenyl, and azulenyl.

In some embodiments, the aryl group includes multiple condensed rings (e.g., naphthyl or anthryl) and aryl groups may be substituted with from 1 to 5 substituents, or 1 to 3 substituents such as hydroxyl, bromo, fluoro, chloro, iodo, mercapto or thio, cyano, cyanoamido, alkylthio, heterocycle, aryl, heteroaryl, carboxyl, carbalkoyl, alkyl, alkenyl, nitro, amino, alkoxyl, amido, acyl, acylamino, acyloxy, alkenyl, substituted alkenyl, alkoxy, substituted alkoxy, alkoxycarbonyl, alkyl, substituted alkyl, alkynyl, substituted alkynyl, substituted amino, aminocarbonyl, aminocarbonylamino, aminocarbonyloxy, aryloxy, azido, carboxyl, cycloalkyl, substituted cycloalkyl, hydroxyl, thioalkoxy, substituted thioalkoxy, thioaryloxy, thiol, alkyl-S(O)—, aryl-S(O)—, alkyl-(O)₂— or aryl-S(O)₂—. In certain embodiments, the aryl group may be further substituted with groups capable of donating NO, such as ONO, ONO₂, SNO, and N(NO)₂.

The term “heteroaryl” includes a ring system including one or more aromatic rings and containing one or more heteroatoms, N, O, or S, in the aromatic ring. Heteroaryl groups can be unsubstituted or may be substituted for example as described for alkyl and aryl groups. Examples of heteroaryl groups include, but are not limited to, pyridinyl, pyrazinyl, pyrimidinyl, benzothialozyl, pyrazolyl, benzoxazolyl, imidazolyl, pyrrolyl, thiadiazolyl, oxazolyl, isoxazolyl, pyridazinyl, triazolyl, thiazolyl, isothiazolyl, thiophenyl, furanyl, and quinolinyl.

The term “acyl” includes groups such as —C(O)R^{a}. In certain embodiments, R^{a} is, for example, hydrogen, substituted or unsubstituted alkyl, substituted or unsubstituted aryl or substituted or unsubstituted cycloalkyl.

The term “acylamino” includes groups such as —NR^{b}C(O)R^{b}. In some embodiments, each R^{b} is independently hydrogen, substituted or unsubstituted alkyl, substituted or unsubstituted aryl or substituted or unsubstituted cycloalkyl.

The term “acyloxy” includes groups such as —OC(O)R^{c}. In certain embodiments, R^{c} may be hydrogen, substituted or unsubstituted allyl, substituted or unsubstituted aryl or substituted or unsubstituted cycloalkyl.

The term “alkenyl” includes a monovalent, branched or unbranched, unsaturated hydrocarbon group (e.g., from 2 to 10 carbon atoms, or 2 to 6 carbon atoms) and having at least 1 (e.g, 1-2) sites of carbon-carbon double bond unsaturation. In some embodiments, the alkenyl groups include ethenyl (—CH═CH₂), n-propenyl (—CH₂CH═CH₂), isopropenyl (—C(CH₃)═CH₂), and the like.

The term “substituted alkenyl” includes alkenyl groups having from, for example, 1 to 5 substituents or 1 to 3 substituents. In some embodiments, the substituents may independently be substituted or unsubstituted acyl, substituted or unsubstituted acylamino, substituted or unsubstituted acyloxy, substituted or unsubstituted alkoxycarbonyl, alkoxycarbonylamino, substituted or unsubstituted amino, aminocarbonyl, aminocarbonylamino, aminocarbonyloxy, substituted or unsubstituted aryl, substituted or unsubstituted aryloxy, azido, carboxyl, cyano, substituted or unsubstituted cycloalkyl, halogen, hydroxyl, keto, nitro, substituted or unsubstituted thioalkoxy, thioaryloxy, thioketo, thiol, alkyl-S(O)—, aryl-S(O)—, alkyl-S(O)₂— and aryl-S(O)₂—.

The term “alkoxy” includes the group —OR^{d} where R^{d} is substituted or unsubstituted alkyl (e.g., 1-10 carbons). In some embodiments, the alkoxy groups may be, for example, methoxy, ethoxy, n-propoxy, isopropoxy, n-butoxy, tert-butoxy, sec-butoxy, n-pentoxy, or n-hexoxy, 1,2-dimethylbutoxy.

The term “substituted alkoxy” includes an alkoxy group having from 1 to 5 substituents (e.g., 1 to 5 substituents), where the substituents may independently include substituted or unsubstituted acyl, substituted or unsubstituted acylamino, substituted or unsubstituted acyloxy, substituted or unsubstituted alkoxy, alkoxycarbonyl, alkoxycarbonylamino, substituted or unsubstituted amino, aminocarbonyl, aminocarbonylamino, aminocarbonyloxy, substituted or unsubstituted aryl, aryloxy, azido, carboxyl, cyano, substituted or unsubstituted cycloalkyl, halogen, hydroxyl, keto, nitro, substituted or unsubstituted thioalkoxy, substituted or unsubstituted thioaryloxy, thioketo, thiol, alkyl-S(O)—, aryl-S(O)—, alkyl-S(O)₂— or aryl-S(O)₂—.

The term “alkoxycarbonyl” includes the group —C(O)OR^{e} where R^{e} may be substituted or unsubstituted alkyl optionally or substituted cycloalkyl.

“Alkoxycarbonylamino” includes the group —NR^{f}C(O)OR^{g}, where R^{f} may be, for example, hydrogen, substituted or unsubstituted alkyl, substituted or unsubstituted aryl or substituted or unsubstituted cycloalkyl, and R^{g} may be, for example, substituted or unsubstituted alkyl or substituted or unsubstituted cycloalkyl.

The term “alkylene” includes divalent branched or unbranched saturated hydrocarbon having, for example, from 1 to 10 carbon atoms. This term is exemplified by groups such as methylene (—CH₂—), ethylene (—CH₂CH₂—), the propylene isomers (e.g., —CH₂CH₂CH₂— and —CH(CH₃)CH₂—) and the like.

The term “substituted alkylene” includes alkylene groups having, for example, from 1 to 5 substituents, where the substituents may independently include, for example, substituted or unsubstituted acyl, acylamino, acyloxy, substituted or unsubstituted alkoxy, alkoxycarbonyl, alkoxycarbonylamino, substituted or unsubstituted amino, aminocarbonyl, aminocarbonylamino, aminocarbonyloxy, substituted or unsubstituted aryl, aryloxy, azido, carboxyl, cyano, substituted or unsubstituted cycloalkyl, halogen, hydroxyl, keto, nitro, substituted or unsubstituted thioalkoxy, substituted or unsubstituted thioaryloxy, thioketo, thiol, alkyl-S(O)—, aryl-S(O)—, alkyl-S(O)₂— or aryl-S(O)₂—.

The term “alkynyl” includes monovalent branched or unbranched unsaturated hydrocarbon groups (e.g., having from 2 to 10 carbon atoms) having at least 1 (e.g., 1, 2) sites of carbon-carbon triple bond unsaturation. Exemplary alkynyl groups include ethynyl (—C≡CH), propargyl (—CH₂C≡CH) and the like.

“Substituted alkynyl” includes alkynyl groups having from 1 to 5 substituents, where the substituents may independently include, for example, substituted or unsubstituted acyl, acylamino, acyloxy, substituted or unsubstituted alkoxy, alkoxycarbonyl, alkoxycarbonylamino, substituted or unsubstituted amino, aminocarbonyl, aminocarbonylamino, aminocarbonyloxy, substituted or unsubstituted aryl, aryloxy, azido, carboxyl, cyano, substituted or unsubstituted cycloalkyl, halogen, hydroxyl, keto, nitro, substituted or unsubstituted thioalkoxy, substituted or unsubstituted thioaryloxy, thioketo, thiol, alkyl-S(O)—, aryl-S(O)—, alkyl-(O)₂— or aryl-S(O)₂—.

The term “substituted amino” includes groups such as —N(R^{h})₂ where each R^{h} may independently be, for example, hydrogen, allyl, substituted allyl, alkenyl, substituted alkenyl, alkynyl, substituted alkynyl, substituted or unsubstituted aryl, cycloalkyl, substituted cycloalkyl, or where the R^{h} groups join to form an substituted or unsubstituted alkylene group. When both R^{h} groups are hydrogen, —N(R^{h})₂ is an amino group.

The term “aminocarbonyl” includes groups such as —C(O)NR^{j}R^{k} where R^{j} and R^{k} may independently be hydrogen, alkyl, aryl and cycloalkyl, or where R^{j} and R^{k} join to form an alkylene group, which is substituted or unsubstituted.

The term “aminocarbonylamino” includes groups —NR¹C(O)NR^{m}Rⁿ where R¹, R^{m}, and Rⁿ may independently be hydrogen, alkyl, aryl and cycloalkyl, or where R^{m} and Rⁿ join to form an alkylene group, which is substituted or unsubstituted.

The term “aminocarbonyloxy” includes groups such as —OC(O)NR^{p}R^{q} where R^{p} and R^{q} may independently be hydrogen, alkyl, aryl and cycloalkyl, or where R^{p} and R^{q} join to form an alkylene group, which is substituted or unsubstituted.

The term “aryloxy” includes groups such as —OR^{S} where R^{S} is an substituted or unsubstituted aryl group.

The term “cycloalkyl” includes cyclic alkyl groups of, for example, 3 to 10 carbon atoms having a single cyclic ring or multiple condensed or bridged ring. The rings may be substituted or unsubstituted with from, for example, 1 to 3 alkyl groups. Such cycloalkyl groups include, by way of example, single ring structures such as cyclopropyl, cyclobutyl, cyclopentyl, cyclooctyl, 1-methylcyclopropyl, 2-methylcyclopentyl, 2-methylcyclooctyl, and the like, or multiple or bridged ring structures such as adamantanyl and the like. The term “lower cycloalkyl” refers to a cycloalkyl group having from 3 to 6 carbon atoms.

“Substituted cycloalkyl” includes cycloalkyl groups having, for example, from 1 to 5 substituents, where the substituents may independently include, for example, substituted or unsubstituted acyl, acylamino, acyloxy, substituted or unsubstituted alkoxy, alkoxycarbonyl, alkoxycarbonylamino, substituted or unsubstituted amino, aminocarbonyl, aminocarbonylamino, aminocarbonyloxy, substituted or unsubstituted aryl, aryloxy, azido, carboxyl, cyano, substituted or unsubstituted cycloalkyl, halogen, hydroxyl, keto, nitro, substituted or unsubstituted thioalkoxy, substituted or unsubstituted thioaryloxy, thioketo, thiol, alkyl-S(O)—, aryl-(O)—, alkyl-S(O)₂— or aryl-S(O)₂—.

“Cycloalkoxy” includes groups —OR^{t} where R^{t} may be, for example, cycloalkyl, as described above. Such cycloalkoxy groups include, by way of example, cyclopentoxy, cyclohexoxy and the like.

“Cycloalkenyl” includes, for example, a cyclic alkenyl group of from 4 to 10 carbon atoms having a single cyclic ring and at least one point of internal unsaturation which can be substituted or unsubstituted with from 1 to 3 alkyl groups. Examples of suitable cycloalkenyl groups include, for instance, cyclopent-3-enyl, cyclohex-2-enyl, cyclooct-3-enyl and the like.

“Substituted cycloalkenyl” includes cycloalkenyl groups having, for example, from 1 to 5 substituents, where the substituents may independently include, for example, substituted or unsubstituted acyl, acylamino, acyloxy, substituted or unsubstituted alkoxy, alkoxycarbonyl, alkoxycarbonylamino, substituted or unsubstituted amino, aminocarbonyl, aminocarbonylamino, aminocarbonyloxy, substituted or unsubstituted aryl, aryloxy, azido, carboxyl, cyano, substituted or unsubstituted cycloalkyl, halogen, hydroxyl, keto, nitro, substituted or unsubstituted thioalkoxy, substituted or unsubstituted thioaryloxy, thioketo, thiol, alkyl-S(O)—, aryl-S(O)—, alkyl-S(O)₂— or aryl-S(O)₂—.

“Thioalkoxy” includes groups —SR^{r} where R^{r} is, for example, substituted or unsubstituted alkyl as described above.

“Substituted thioalkoxy” includes thioalkoxy group having, for example, from 1 to 5 substituents, where the substituents may independently include, for example, substituted or unsubstituted acyl, acylamino, acyloxy, substituted or unsubstituted alkoxy, alkoxycarbonyl, alkoxycarbonylamino, substituted or unsubstituted amino, aminocarbonyl, aminocarbonylamino, aminocarbonyloxy, substituted or unsubstituted aryl, aryloxy, azido, carboxyl, cyano, substituted or unsubstituted cycloalkyl, halogen, hydroxyl, keto, nitro, substituted or unsubstituted thioalkoxy, substituted or unsubstituted thioaryloxy, thioketo, thiol, alkyl-S(O)—, aryl-S(O)—, alkyl-S(O)₂— or aryl-S(O)₂—.

“Thioaryloxy” includes the group —SR^{u} where R^{u} is substituted or unsubstituted aryl, for example, as described above.

In particular embodiments, the ROS scavenger component may be an N-oxide free radical, wherein optionally the nitrogen of the N-oxide free radical is within a 3-, 4-, 5-, 6- or 7-membered ring, wherein the ring may be substituted or unsubstituted with, for example, straight or branched chain C₁-C₇, or C₁-C₃ alkyl groups, alkoxy groups and groups capable of donating NO.

The N-oxide free radical is preferably substituted. In particular embodiments the N-oxide free radical is fully substituted at positions alpha to the nitroxide free radical, and may optionally be substituted at other positions on the ring. Exemplary substituents for the alpha positions include methyl or ethyl. Exemplary substituents for other ring positions include NO donor groups.

The nitrogen of the substituted N-oxide free radical may also be linked to the beta-antagonist at the backbone amine of the beta-antagonist (e.g., R^{5A} or R^{5B} of Formula I).

In certain other embodiments the substituted N-oxide free radical may also be substituted within the ring with an additional heteroatom, for example, —O— or —S—, (see structures Ia and Ib, below). Exemplary substituted N-oxide free radicals include substituted pyrrolidinyloxy free radicals (e.g., PROXYL), substituted piperidinyloxy free radicals (e.g., TEMPO), substituted oxazolidinyloxy free radicals (e.g., DOXYL), substituted oxazinyloxy free radicals, substituted thiazolidinyloxy free radicals and substituted thiazinyloxy free radicals.

In certain embodiments, the ROS scavenger(s) may be independently selected from the group consisting of substituted piperidinyloxy free radical, substituted 3-pyrrolidin-1-yloxy free radical, substituted oxazolidinyloxy free radical (e.g., DOXYL), and an substituted or unsubstituted lipoic acid moiety.

Examples of substituted N-oxide free radical moieties which may be incorporated into the multifunctional β-antagonist compounds include a 2,2,6,6-tetramethylpiperidinyloxy free radical (TEMPO) moiety (Ia, below, where X═C), a 2,2,5,5-tetramethyl-3-pyrrolidin-1-yloxy free radical (PROXYL) moiety (Ib, below, where X═C); 4,4-dimethyl-3-oxazolidinyloxy (DOXYL) free radical moiety, and a 2,2,4,4-tetramethyl-3-oxazolidinyloxy free radical moiety (Ib, below, where X═O). In structures Ia-f below, X is for example —S—, —C— or —O—. The substituted N-oxide free radical moiety may be linked to the β-antagonist moiety for example, directly, indirectly, via a linker (e.g., through an alkyl substituent group, see, for example Ic and Id), and/or via sharing of atoms, for example as shown in structures Ie and If below. The linkage may be to various carbon atoms on the ring, including those shown in structures Ic-If below. Additionally, the substituted N-oxide free radical moiety may be linked to the beta-antagonist component via incorporation in a fused ring system.

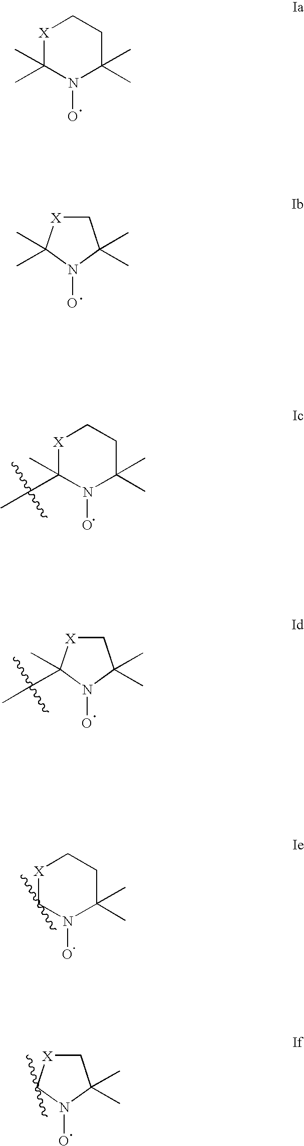

In other embodiments the ROS scavenger component comprises a lipoic acid moiety or may be derived from the lipoic acid moiety. The lipoic acid moiety may be substituted or unsubstituted and is shown below: 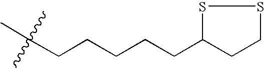
The lipoic acid moiety may be independently substituted by one or more groups such as straight or branched chain C₁-C₁₅ alkyl groups, C₁-C₁₅ alkoxy groups, hydroxy groups, amino groups (NH₂), —NHCHO groups, —CH₂OH groups, and groups capable of donating NO in a charged or neutral form.

In other embodiments, the ROS scavenger component may be a pantothenic acid SH-containing derived moiety as shown below, in either an oxidized or reduced form: 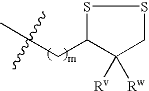
wherein, m is for example, 1-6, and R^{v} and R^{w} are for example independently C₁-C₃ alkyl or H.

In other embodiments, the lipoic acid moiety may be modified by varying the length of the aliphatic chain connecting the heterocyclic ring to the β-antagonist component of the multifunctional β-antagonist compound. The chain may be for example (CH₂)ₙ wherein n is an integer from 1-15. In particular embodiments, n is 3 or 12 as shown below.

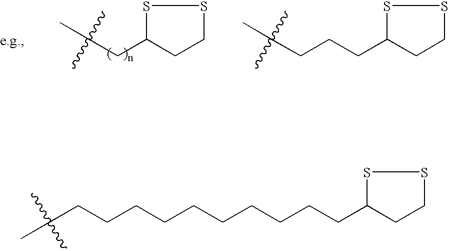

In other embodiments, the ROS scavenger/SOD mimic component may be a component as shown below, comprising an aromatic ring (e.g., phenyl), which may be part of the beta-antagonist component, fused with a substituted N-oxide free radical component, where the nitrogen of the N-oxide free radical is contained with a cyclic or heterocyclic ring (e.g., a 5-, 6-, or 7-membered ring); substituted or unsubstituted S—S-containing ring (e.g., a 5-, 6-, or 7-membered ring); or a substituted or unsubstituted aromatic heterocycle (e.g., a 5-, 6-, or 7-membered ring, such as furan or pyrrole).

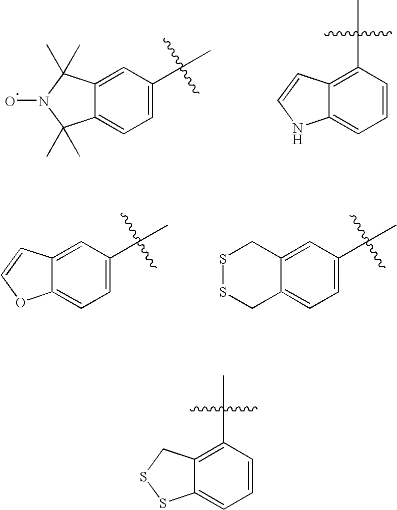

The ROS scavenger/SOD mimic component may also comprise a substituted N-oxide free radical, where the nitrogen of the N-oxide free radical is contained with a cylic ring (e.g., a 5-, 6-, or 7-membered ring) and is linked to the beta-antagonist at the backbone amine of the beta-antagonist component (e.g., R^{5A}/R^{5B} of Formula I). Exemplary N-oxide free radicals are shown below, where the NH as pictured below 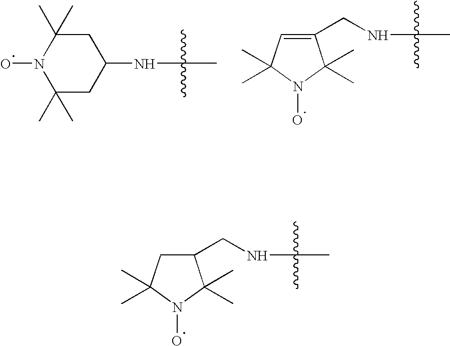
may form part of the beta-antagonist component.

In some embodiments, the ROS scavenger/SOD mimic component may comprise a substituted or unsubstituted S—S-containing ring (e.g., 5-, 6-, or 7-membered ring), e.g., as shown below, where the NH as pictured below may form part of the beta-antagonist component

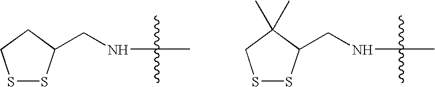

In certain embodiments the ROS scavenger component, including those described above, may be independently substituted with one or more alkyl groups such as C₁-C₁₅ alkyl groups, alkoxy such as C₁-C₁₅ alkoxy groups, hydroxy groups, amino groups including NH₂, —NHCHO groups, —CH₂OH groups, and groups capable of donating NO in a charged or neutral form.

In particular embodiments, the ROS scavenger component(s) comprises one or more PROXYL moieties, one or more TEMPO moieties, one or more DOXYL moieties, one or more 2,2,4,4-tetramethyl-3-oxazolidinyloxy free radical moieties and/or one or more substituted or unsubstituted lipoic acid moieties. In particular embodiments the groups comprising N-oxide free radical moieties are independently substituted by one or more C₁-C₃ alkyl groups, for example methyl, ethyl or butyl, or one or more C₁-C₃ alkoxy groups.

The multifunctional β-agonist compounds may be modified to include one or more of the same or different SOD mimic component and/or ROS scavenger component.

βAntagonists

The β-antagonist component of any of a variety of β-antagonist compounds for the treatment of cardiovascular and other conditions disclosed herein can be present in the multifunctional β-antagonist compounds. In one embodiment, a known β-antagonist is provided in multifunctional form which further includes at least one NO donor component and at least one ROS scavenger component. The β-antagonist compound or component is one that is capable of blocking β-adrenoreceptors. Once incorporated into the multifunctional β-antagonist compounds, the multifunctional β-antagonist compounds may be used to treat any of the indications for which treatment with β-antagonist is indicated.

Exemplary β-antagonists include compounds used in the treatment of cardiovascular conditions and others described herein that selectively block the βadrenergic receptors. β-Adrenergic receptor antagonist agents (also referred to as “β-blockers”, “β-blocking agents”, “β-receptor blocking agents” or “β-antagonists”) remain the cornerstone for therapy of all stages of ischemic heart disease. They constitute the standard therapy for effort angina, mixed effort and rest angina, and unstable angina. They decrease mortality in acute-phase myocardial infarction and in the post-infarct period. In addition to their primary role in the treatment of ischemic heart disease, β-blockers retain their leading position among basic therapies for other cardiovascular conditions including hypertension, arrhythmias, cardiomyopathy, and congestive heart failure. β-Blockers also possess other properties that turn them useful for the treatment of non-cardiovascular conditions. These include, but are not limited to, central indications (e.g., anxiety, essential tremor, migraine prophylaxis, alcohol withdrawal), endocrine conditions (e.g., thyrotoxicosis), gastrointestinal (e.g., esophageal varices), and ocular conditions (e.g., topical use for glaucoma).

Situated on the cardiac sarcolemma, the β₁-receptor is part of the adenyl cyclase system. The G-protein system links the receptor to adenyl cyclase, when the G-protein is in the stimulatory configuration. The link is interrupted by the inhibitory form of the G-protein, the formation of which results from muscarinic stimulation following vagal activation. When activated, adenyl cyclase produces cyclic AMP (cAMP), which is the intracellular second messenger of β₁-stimulation. Cyclic AMP mediates several signaling pathways, among which is the “opening” of calcium channels to promote a positive inotropic effect and increased re-uptake of cytosolic calcium into the sarcoplasmic reticulum (relaxing or lusitropic effect). In the sinus node, the pacemaker current is increased (positive chronotropic effect), and the rate of conduction is accelerated (positive dromotropic effect). The effect of a given β-blocking agent depends not only on the way it is absorbed, bound to plasma proteins, and on its metabolism, but also on the extent to which it inhibits the β-receptor (lock-and-key fit). Some blockers also have the capacity to activate the receptor, hence the term partial agonist activity (PAA), also called intrinsic sympathomimetic activity (ISA), as epitomized in pindolol. ISA tends to avoid resting bradycardia and confers some vasodilatory activity on the β-blocker.

During β₁-adrenergic stimulation, the increased contractile activity, resulting from the greater and faster rise of cytosolic calcium, is coupled to increased breakdown of ATP by the myosin ATPase and increased formation of cAMP. Thus, the uptake of calcium is enhanced with a more rapid rate of fall of cytosolic calcium, thereby accelerating relaxation. Increased cAMP also increases the phosphorylation of troponin-1, so that the interaction between the myosin heads and actin ends more rapidly. Therefore, the β-blocked heart not only will beat more slowly by inhibition of the depolarizing currents in the sinoatrial node, but also will have a decreased force of contraction and a decreased rate of relaxation.

β-Blockers were originally designed to counteract the effects of increased adrenergic stimulation. By blocking the cardiac β-receptors, these agents induce the well-known inhibitory effects on sinus node, atrioventricular node, and on myocardial contraction. These are, respectively, the negative chronotropic, dromotropic, and inotropic effects. Of these, it is especially the bradycardia and the negative inotropic effects of β-blockers that are relevant to their therapeutic effect in angina pectoris, because these changes decrease the myocardial oxygen demand. The longer diastolic filling time, resulting from the decreased heart rate in exercise, leads to better diastolic myocardial perfusion, which is an important therapeutic benefit. The inhibitory effect on the AV node is of special relevance in the therapy of supraventricular tachycardias, or when β-blockade is used to control the ventricular response rate in atrial fibrillation.

The above effects explain why β₁-blockers are anti-anginal, as predicted by their developers. Antihypertensive effects are less well understood. In the absence of the peripheral dilatory action of some agents, β-blockers initially decrease the resting cardiac output by about 20%, with a compensatory reflex rise in the peripheral vascular resistance. Thus, within the first 24 hours of therapy, the arterial pressure is unchanged. The peripheral resistance starts to fall after 1 to 2 days, and the arterial pressure declines. The mechanism of this hypotensive process is unclear, but may involve inhibition of β-receptors on the terminal neurons that facilitate the release of norepinephrine; central nervous system effects with reduction of adrenergic outflow; lessening of the activity of renin-angiotensin system, because β₁-receptors mediate rennin release.

β-blockers are now recognized as an integral part of anti-heart failure therapy. However, despite the increasingly impressive results of β-blocker therapy in heart failure, the mechanism(s) of action is still unclear. Several mechanisms may be involved, as described below.

β-Blockade may act, at least in part, by reduction of heart rate. Bradycardia may improve coronary blood flow and decrease the myocardial oxygen demand.

The circulating concentrations of norepinephrine found in severe heart failure are high enough to be directly toxic to the myocardium, resulting in damaging the membrane and promoting subcellular destruction. Arrhythmias are promoted via increased formation of cAMP. The impressive beneficial effect of β-blockers may therefore be due to their ability to protect cells from catecholamine toxicity.

In advanced heart failure in humans, there is prominent down-regulation of the β₁-adrenergic receptor and its signaling pathways. Prolonged excess stimulation leads to increased activity of the β₁-adrenergic receptor kinase (β₁-ARK) that in turn phosphorylates and inhibits the β₁-receptors to decrease contractile activity. Treatment with β₁-blockers decreases the expression of β₁-ARK and increases the activity of adenyl cyclase. It is hence concluded that β₁-blockers own their beneficial effect in the treatment of heart failure to re-sensitization of the down-regulated receptor system, thereby improving contractile function.

Another explanation for the benefits of β-blockade in heart failure stems from the current recognition that the coupling of the β₂-receptor to the inhibitory G-protein may be anti-apoptotic. A role for apoptosis in the progression of heart failure, although controversial, is increasingly postulated. Anti-apoptosis is new albeit still hypothetical therapeutic principle in heart failure. β₁-blockade could therefore leave unopposed the protective β₂-receptor pathway.

When added to prior to ACE inhibitor or angiotensin receptor blocker therapy, β₁-blockade decreases circulating rennin and angiotensin II levels, increasing the blockade of the rennin-angiotensin system, which is considered as an integral part of the anti-heart failure properties of β₁-receptor blockers.

β₁-Receptor blockers many also be used for many other non-cardiac indications, as described herein.

Beta-antagonists may also be used in conjunction with vascular surgery. Perioperative deaths from cardiac causes and myocardial infarction were reduced by use of β-blockers in high-risk patients undergoing vascular surgery.

Beta-antagonists have also been successfully used in the treatment of thyrotoxicosis. Used together with antithyroid drugs or radioiodine, or as the sole therapy before surgery, β-blockade is commonly used in thyrotoxicosis to control symptoms, although the hypermetabolic state is not decreased. β-Blockade controls tachycardia, palpitations, tremor, and nervousness and reduces the vascularity of the thyroid gland, thereby facilitating operation.

Beta-antagonists have also been successfully used in the treatment of anxiety states. Although propranolol is most widely used in anxiety (and licensed for this purpose in several countries, including the United States), probably all β-blockers are effective, acting not centrally but by a reduction of peripheral manifestations of anxiety such as tremor and tachycardia. In a double-blind study of anxiety in hypertensive patients, atenolol was considerably better than propranolol.

Beta-antagonists have also been successfully used in the treatment of other CNS indications. β-Blockers are very effective in postalcoholic withdrawal syndrome. In subarachnoid hemorrhage, early treatment by β-blockers with long-term follow-up appeared beneficial. Patients taking β-blockers at the start of the stroke appeared to have some protection.

Propranolol (licensed in the United States) acts prophylactically to reduce the incidence of migraine attacks in 60% of patients. The mechanism is presumably by beneficial vasoconstriction. The use of timolol for the prophylaxis of migraines is also known in the art.

The use of local β-blockers is now established for open-angle glaucoma. Among the agents approved for treatment of glaucoma in the United States are the nonselective agents timolol, carteolol, levobunolol, betaxol and metipranolol. The cardioselective betaxolol may be an advantage in avoiding side effects in patients with bronchospasm. Thus, in certain embodiments, the invention relates to multifunctional β-antagonist compounds for the treatment of ocular conditions (e.g., glaucoma).

In some embodiments, beta-antagonists which may be used to provide the beta-antagonist component of the multifunctional beta-antagonist compounds described herein may include Acebutolol, Alprenolol (Aptin), Amosulalol, Arotinolol, Atenolol (Atehexal), Befunolol, Betaxolol, Bevantolol, Bisprolol (Zebeta), Bopindolol, Bucumolol, Bufetolol, Bufuralol, Bunitrolol, Bupranolol, Butidrine Hydrochloride, Butofilolol, Carazolol, Carteolol, Carvedilol (Coreg, Dilatrend, Kredex), Carvidilol, Celiprolol, Cetamolol, Cloranolol, Dilevalol, Disopryamide (Norpace), Epanolol, Esmolol, Indenolol, Labetalol, Levobunolol, Mepindolol, Metipranolol, Metohexal (Meijoprolol), Metopolol (Betaloc), Metoprolol, Moprolol, Nadolol, Nadoxolol, Nebivolol, Nifenalol, Nipradilol, Oxprenolol (Corbeton), Penbutolol, Pindolol, Practolol, Pronethalol, Propranolol, Quinidine Gluconate (Quinaglute), Quinidine Polylgalacturonate (Cardioquin), Quinidine Sulfate (Quinidex, Cin-quin), Sotalol (Sotocor, Sotahexal), Sulfinalol, Talinolol, Tertatolol, Tilisolol, Timolol, Toliprolol, Toprol XL, or Xibenolol.

In certain embodiments, the beta-antagonists which may be used to provide the beta-antagonist component of the multifunctional beta-antagonist compounds described herein may include timolol, carteolol, levobunolol, betaxol and metipranolol. These beta-antagonists may be of particular use as beta-antagonist components for the treatment of ocular conditions with the multifunctional beta-antagonists as described herein. The cardioselective betaxolol may be used advantageously in avoiding side effects in patients with bronchospasm, and cardiovascular conditions (e.g., hypertension, arrhythmias, cardiomyopathy and congestive heart failure) other conditions.

In some embodiments, the beta-antagonists which may be used to provide the beta-antagonist component of the multifunctional beta-antagonist compounds described herein may include propranolol or timolol. These beta-antagonists may be of particular use as beta-antagonist components for the treatment of migraines with the multifunctional beta-antagonists as described herein.

Multifunctional β-Antagonist Compounds

The multifunctional β-antagonist compound may include a β-antagonist component, at least one antioxidant component such as a reactive oxygen species (ROS) scavenger (e.g., a SOD mimic), and at least one NO-donor component. The multifunctional β-antagonist compound may include a β-antagonist component linked to at least one NO-donor component and at least one antioxidant component. The term “linked” as used herein is intended to include direct and indirect linkages and shared atoms (including, for example, where the nitrogen of the substituted N-oxide free radical is part of a fused ring system) between any of the NO donor component, antioxidant component, such as ROS scavenger component, and β-antagonist component. The components may be linked in any order, for example, the ROS scavenger component may be linked to both the NO donor component and the β-antagonist component, or the ROS scavenger component may be linked only to the β-antagonist component while the β-antagonist component is also linked to the NO-donor component (e.g., according to Formula I or II).

In some embodiments, functionalized β-receptor blocking compounds are provided that include at least one ROS scavenger component (e.g., SOD mimic) linked to a β-receptor blocking component, which can be made and used as described herein for multifunctional β-receptor blocking compounds.

Also included within the scope of the invention are salts of the compounds disclosed herein and stereoisomers thereof. The compounds of the present invention contain one or more asymmetric atoms and may exist in diastereomeric, racemic and optically active forms. All such compounds and compositions comprising these compounds are contemplated to be within the scope of this invention. Therefore, where a compound is chiral, the separate enantiomers, substantially free of the other, are included within the scope of the invention. Thus, one enantiomer may be, for example, in 95% or higher purity. Further included are all mixtures of enantiomers or diastereomers.

Optically active forms of the compounds can be prepared using any method known in the art, including by resolution of the racemic form by recrystallization techniques, by chiral synthesis, extraction with chiral solvents, or by chromatographic separation using a chiral stationary phase. Examples of methods to obtain optically active materials include transport across chiral membranes, a technique whereby a racemate is placed in contact with a thin membrane barrier. The concentration or pressure differential causes preferential transport across the membrane barrier. Separation occurs as a result of the non-racemic chiral nature of the membrane which allows only one enantiomer of the racemate to pass through. Chiral chromatography, including simulated moving bed chromatography, is used in one embodiment. A wide variety of chiral stationary phases are commercially available.

Since superoxide anion is an available and continuously-formed by-product generated through normal metabolic processes, and since its elimination is mediated either by dismutation by the enzyme SOD or via its reaction with NO to form the potentially hazardous peroxynitrite, without being limited to any theory, the compounds are believed to be capable of simultaneously and favorably affecting both components, NO and O₂⁻. By virtue of the β-antagonist activity, NO donation and superoxide scavenging properties being simultaneously delivered by the same molecule, the compounds of the present invention can increase the level of NO and reduce levels of superoxide thereby avoiding high levels of peroxynitrite and oxidant metabolites thereof and consequently increasing the effectiveness of the beta-antagonist component. Furthermore, the multifunctional beta-antagonist compounds as described herein can also be administered at more predictable doses compared to non-functionalized beta-antagonists due to the ability of the ROS scavenger component to scavenge ROS species which can interfere with beta-antagonist activity or NO donated in vivo.

Therefore one embodiment of the invention provides multifunctional beta-antagonist compounds comprising a functionalized β-receptor blocking component which contains at least one moiety that affords SOD-mimic and/or ROS scavenger activity, and at least one ONO, SNO, or ONO₂ component that confers on the ROS scavenger-β-blocker an additional relaxant effect with all other beneficial biological actions expected from an NO-donor. In other embodiments, functionalized β-receptor blocking compounds are provided that include at least one ROS scavenger and/or SOD mimic component linked to a β-receptor blocking component, which can be made and used as described herein for multifunctional β-receptor blocking compounds.

The multifunctional β-antagonist compound includes in one embodiment, a β-antagonist component; an antioxidant component, such as a reactive oxygen species scavenger (e.g., SOD mimic); and a nitric oxide donor component

Thus, in one embodiment, a β-antagonist is provided in modified form and includes a reactive oxygen species (ROS) scavenger component and a nitric oxide donor component capable of releasing NO in a charged or neutral form. The β-antagonist component may be linked to at least one reactive oxygen species (ROS) scavenger component and at least one nitric oxide donor component.

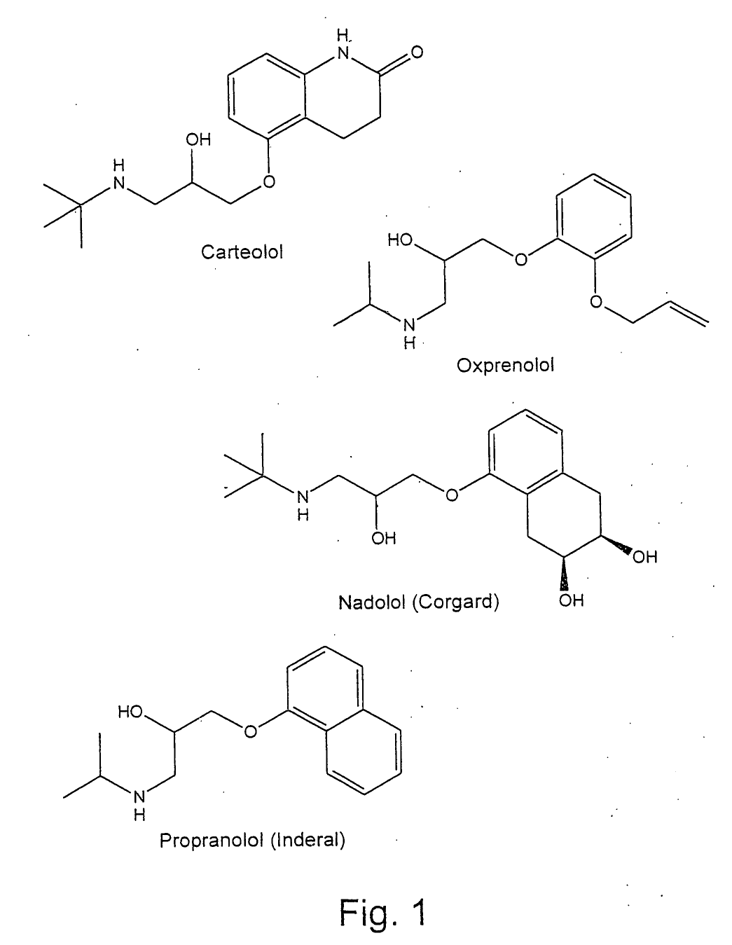

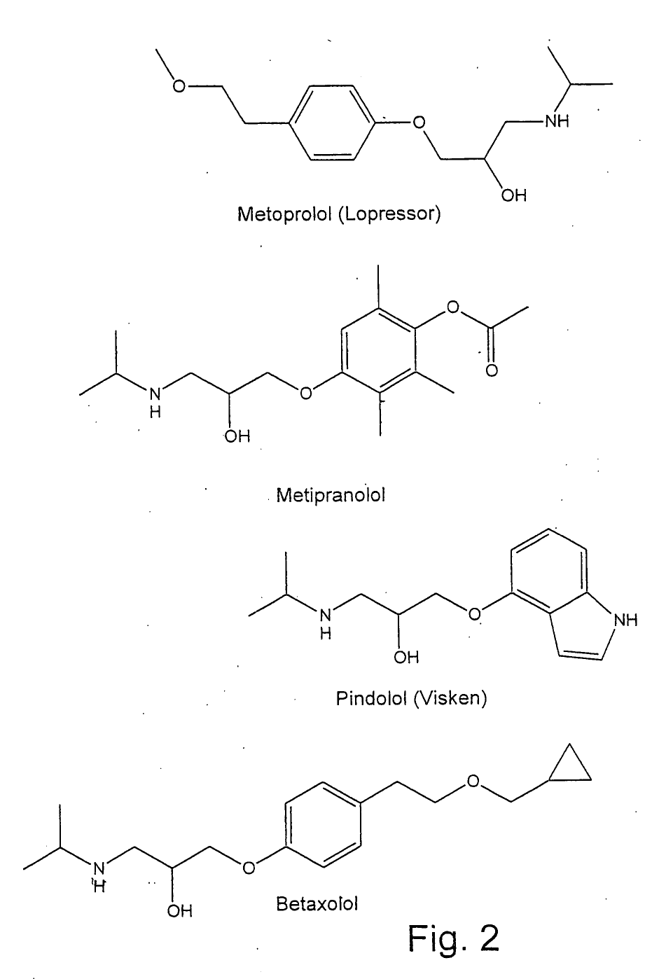

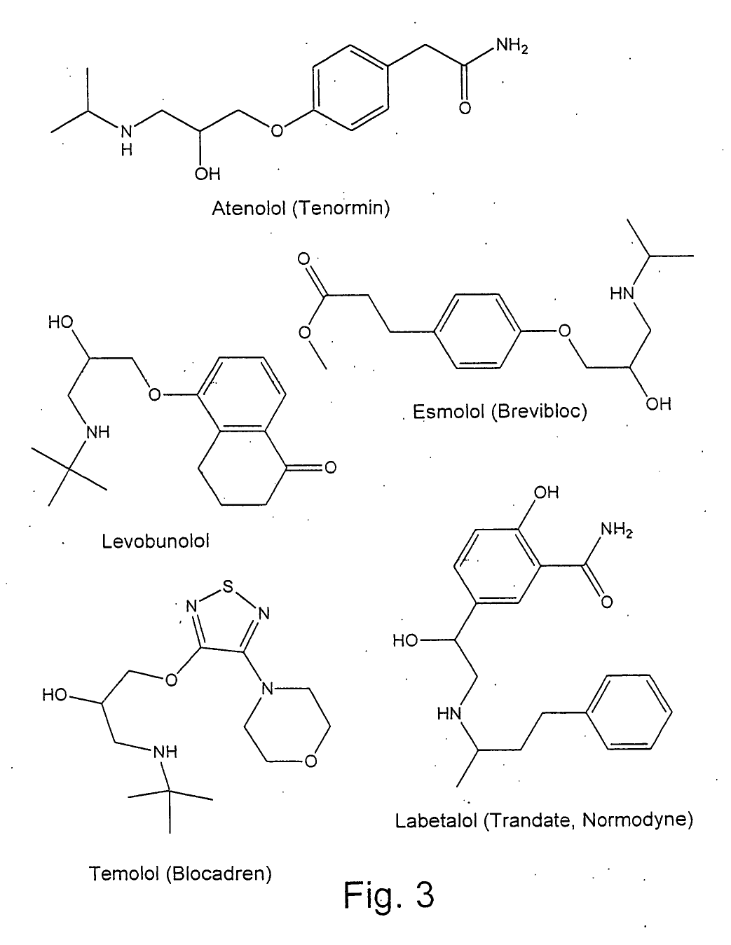

Exemplary β-antagonists which may be modified to provide the beta-antagonist component include those described herein. Certain exemplary beta-antagonist structures from which beta-antagonist components may be selected are also shown in FIGS. 1-3.

In some embodiments, the beta-antagonist component is a propranolol, metipranolol or timolol.

In some embodiments the at least one ROS scavenger component may be a SOD mimic.

The nitric oxide donor components may include —ONO, —ONO₂, —SNO and —NONOate.

The antioxidant component, such as a ROS scavenger component is, for example, a substituted N-oxide free radical, wherein the nitrogen of the N-oxide is contained within a ring (e.g., a 5-, 6-, or 7-membered ring); alkenyl group; aryl group; substituted aryl group, where the aryl group is substituted with, for example, —OH, —NH₂, —NHCHO or a NO donor group; or a group that is, or is capable of being converted in vivo into, a sulfhydryl in oxidized or reduced form (e.g., a group incorporating a lipoic acid moiety).

In some embodiments, are provided novel multifunctional beta-antagonist compounds comprising a beta-antagonist component, at least one NO-donor component and at least one superoxide ion (O₂⁻) scavenger component and their use as therapeutic agents for the treatment of glaucoma, cardiovascular conditions and other conditions in which treatment with beta-antagonists is indicated without producing undesired side effects

In certain embodiments, the beta-antagonist component may be betaxol, carteolol, levobunalol, metipranolol or timolol. In certain embodiments the beta-antagonist component is timolol.

In other embodiments, the beta-antagonist component may be propranolol or timolol. In certain embodiments the beta-antagonist component is timolol.

In other embodiments, functionalized β-antagonist compounds are provided that include at least one ROS scavenger component linked to a β-receptor blocking component, which can be made and used as described herein for multifunctional β-receptor blocking compounds.

Consequently, the present invention relates to β-receptor blocking agents with either SOD (e.g., Formula I) or anti-ROS (e.g., Formula II) activity possessing NO donation properties of the general Formulae I and II. The anticipated superior beneficial therapeutic effects of compounds comprising these Formulae may be attributed to their simultaneous multi-mechanistic actions as β-blockers (see diverse pharmacological actions above), SOD-mimics/anti-ROS (antioxidant and anti-inflammatory that provide additional cellular protection), and as NO-donors (vasodilator, antioxidant, anti-proliferative, cellular protectant with potent vascular smooth muscle relaxing properties). These properties are most needed for adequate prevention and/or treatment of cardiovascular conditions involving ischemia, hypertension, arrhythmias, cardiomyopathy, congestive heart failure, as well as other conditions for which the use β-receptor blocking agents has proven beneficial (anxiety, tremor, migraine, alcohol withdrawal, thyrotoxicosis, esophageal varices, and glaucoma).

In particular, the invention relates to nitrosated or nitrosylated β-receptor blocking agents with SOD-mimic/Anti-ROS actions which can optionally be substituted with at least one ONO, SNO, or ONO₂ moiety, or a compound that donates, transfers, or releases nitric oxide in either a neutral or a charged form.

The suggested compounds offer a new strategy for the treatment of various diseases that can affect not only the clinical symptoms of the disease, but also its pathogenesis, natural course and outcome.

Therefore one embodiment of the invention provides compounds comprising a functionalized β-receptor blocking agents which contain at least one moiety that affords ROS scavenger and/or SOD mimic activity, and at least one ONO, OSNO, or ONO₂ moiety that confers on the resulting ROS scavenger/SOD-mimic-β-blocker an additional relaxant effect with all other beneficial biological actions expected from an NO-donor.

In some embodiments, the ROS scavenger may be a SOD mimic (e.g., substituted pyrolidinyloxy N-oxide free radical); at least one NO donor group is ONO, SNO or ONO₂; and the beta-antagonist component can be from timolol, carteolol, levobunolol, betaxol, metipranolol, or propranolol.

In certain embodiments, the ROS scavenger may be a SOD mimic (e.g., substituted pyrolidinyloxy N-oxide free radical); at least one NO donor group is ONO, SNO or ONO₂; and the beta-antagonist component can be from timolol, carteolol, levobunolol, betaxol, or metipranolol.

In some embodiments, the ROS scavenger may be a SOD mimic (e.g., substituted pyrolidinyloxy N-oxide free radical); at least one NO donor group is ONO, SNO or ONO₂; and the beta-antagonist component can be from propranolol.

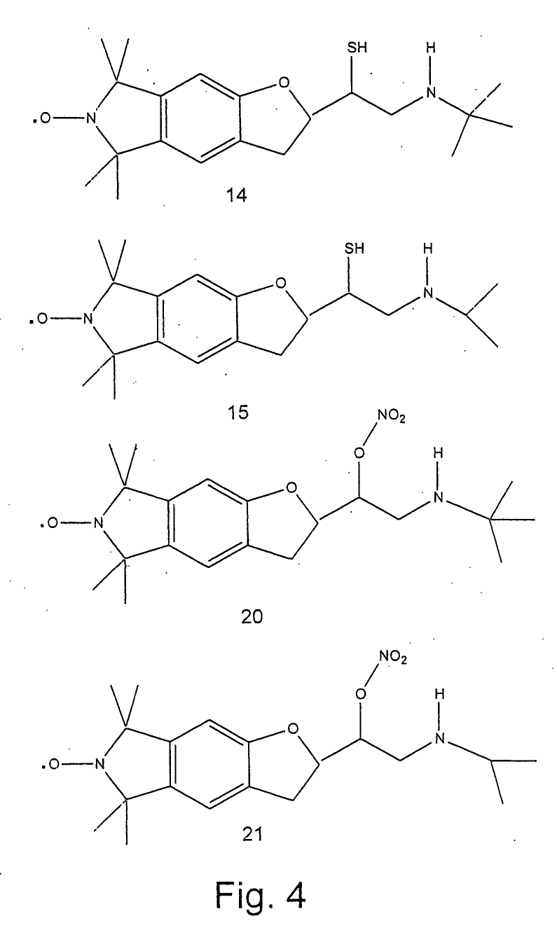

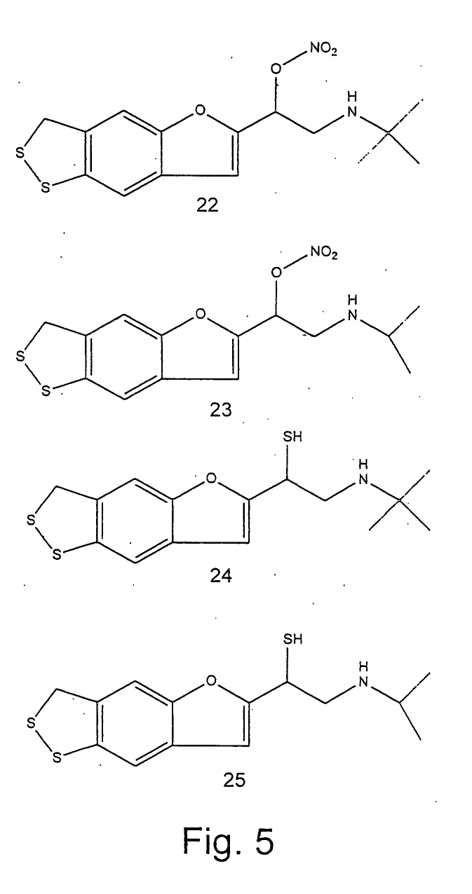

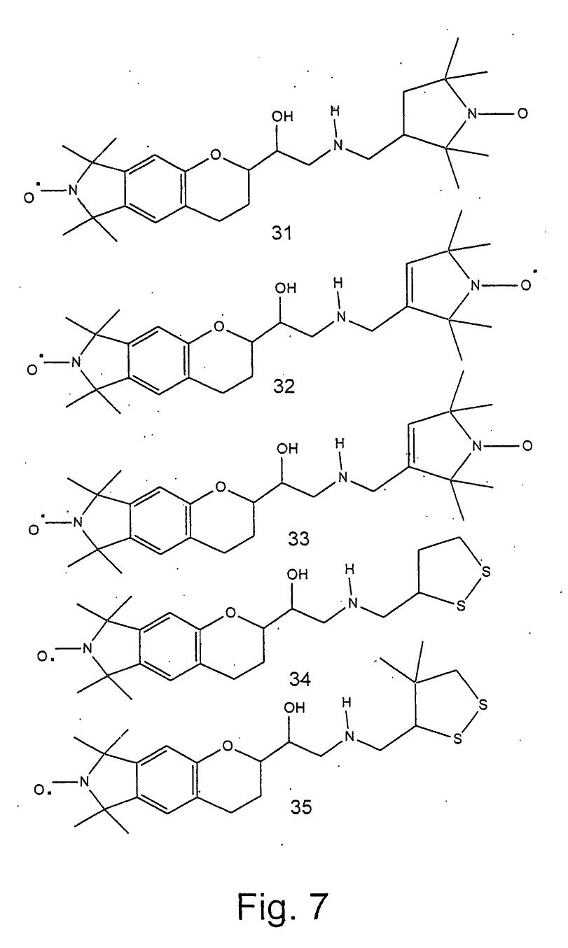

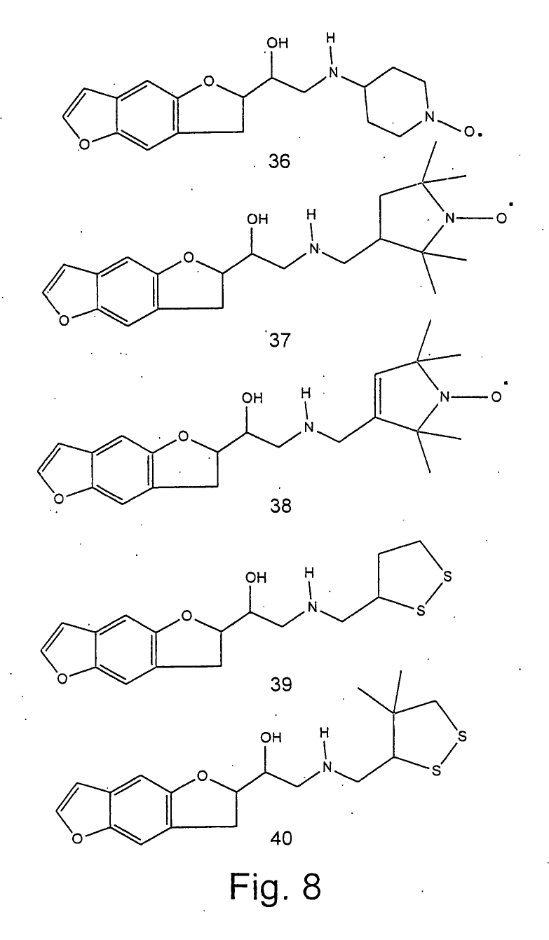

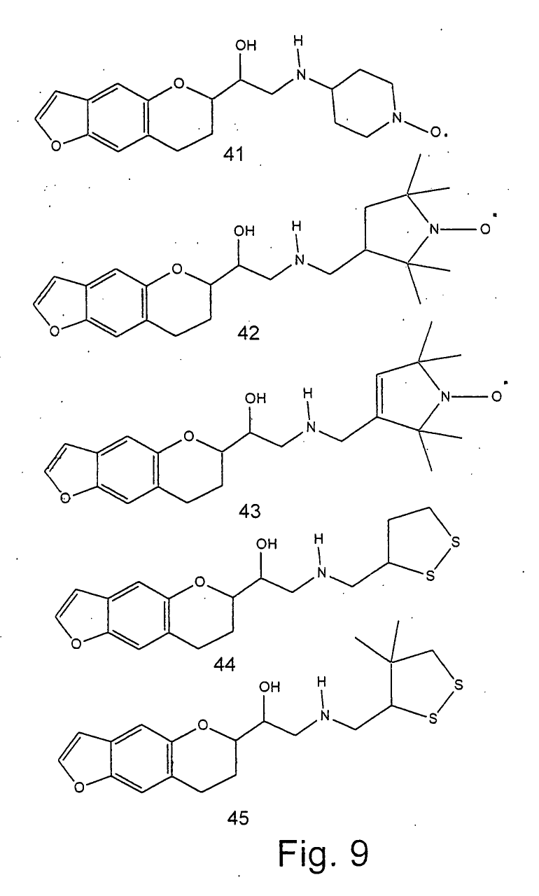

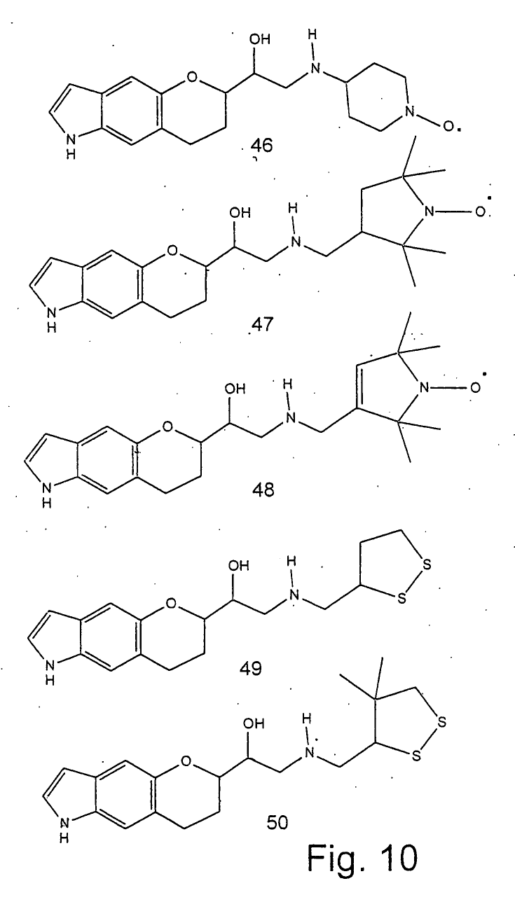

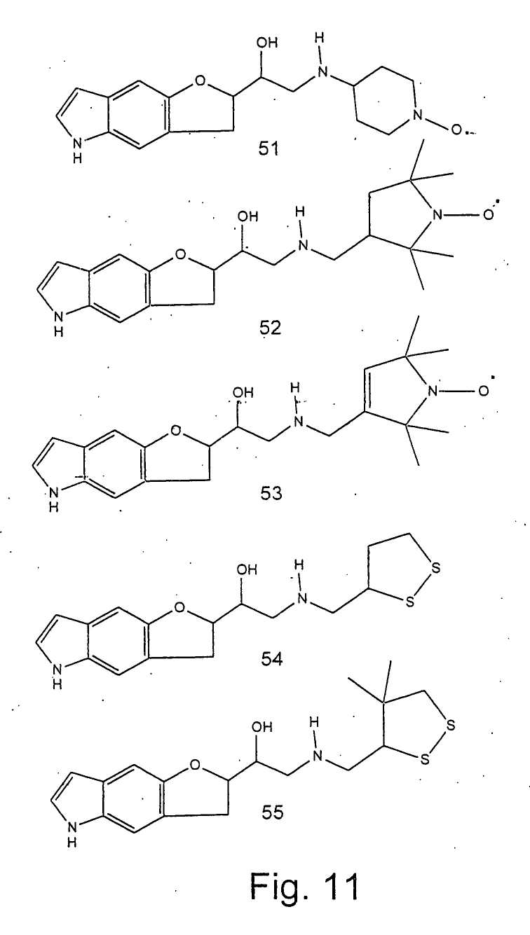

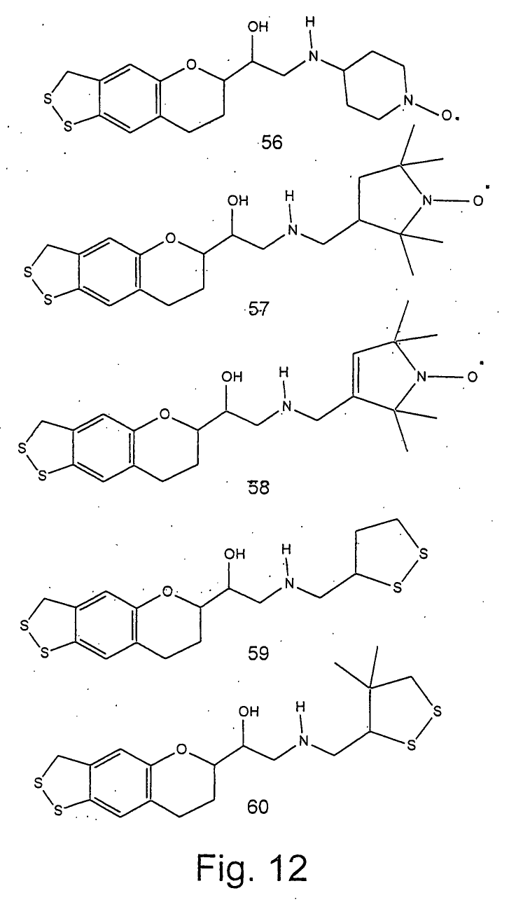

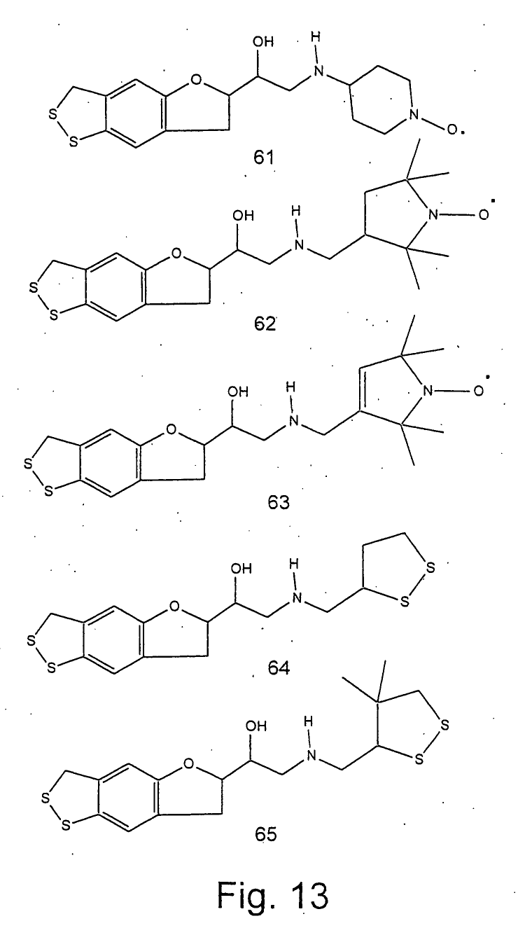

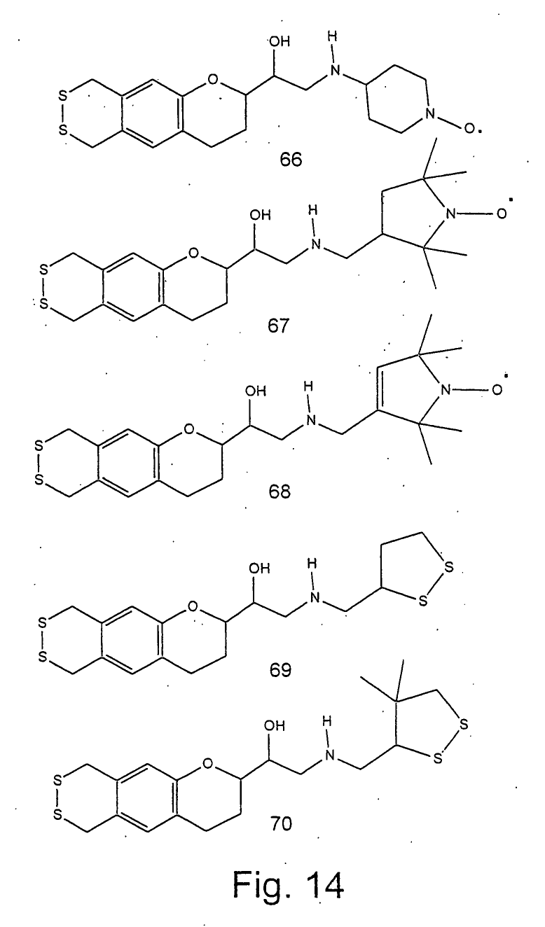

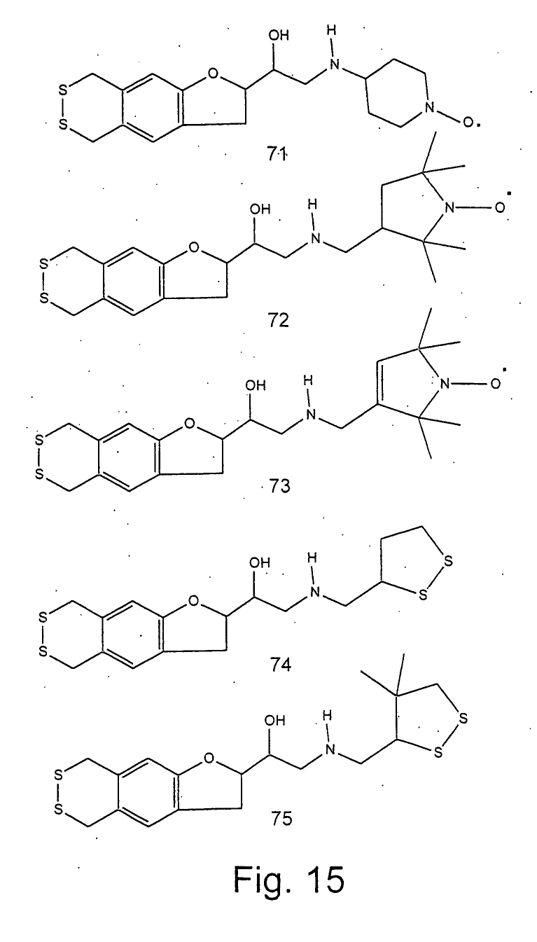

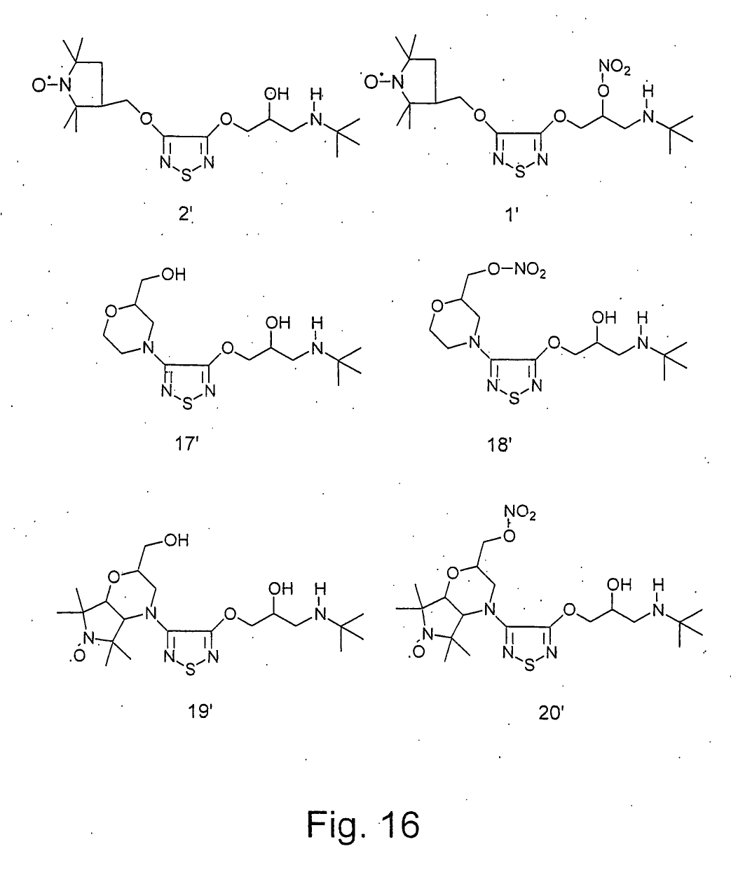

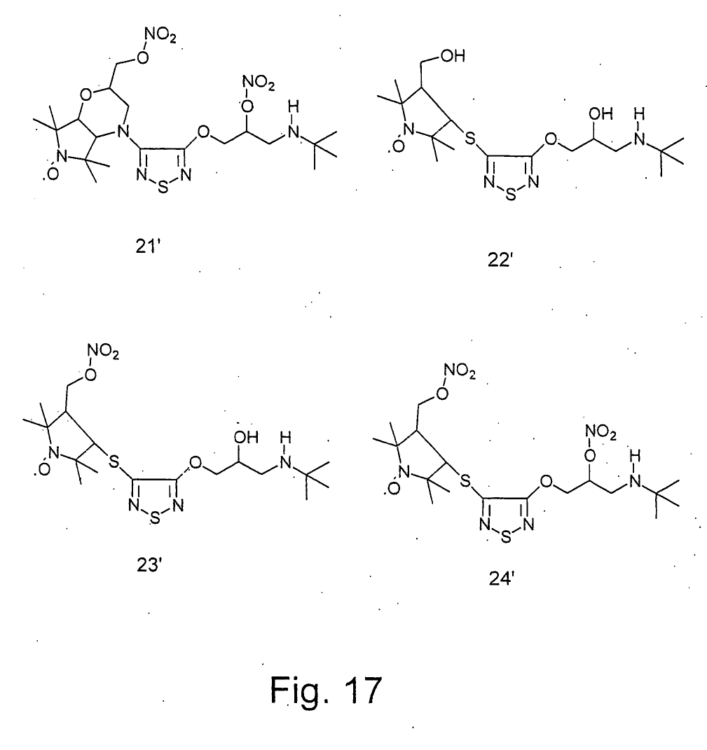

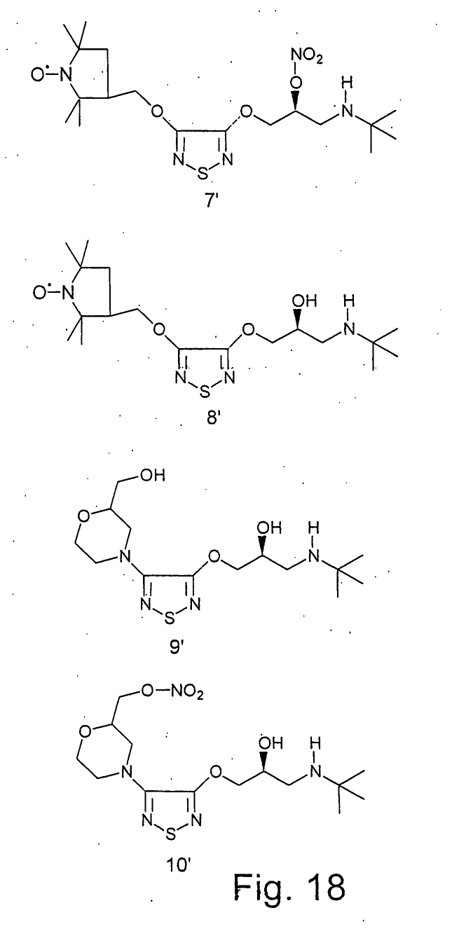

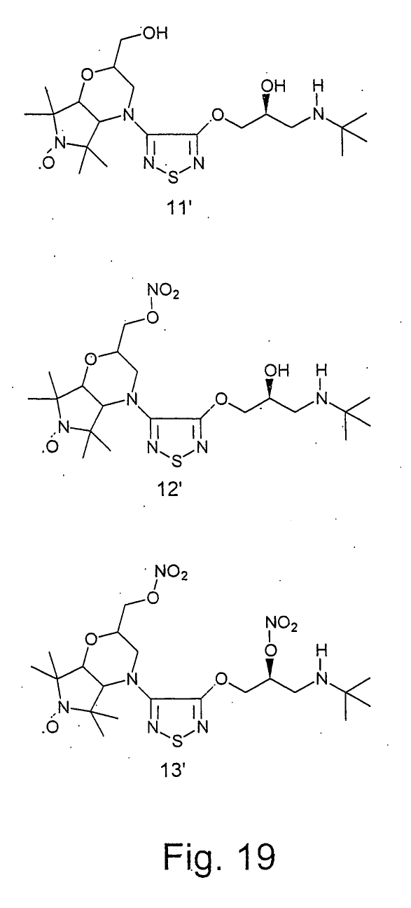

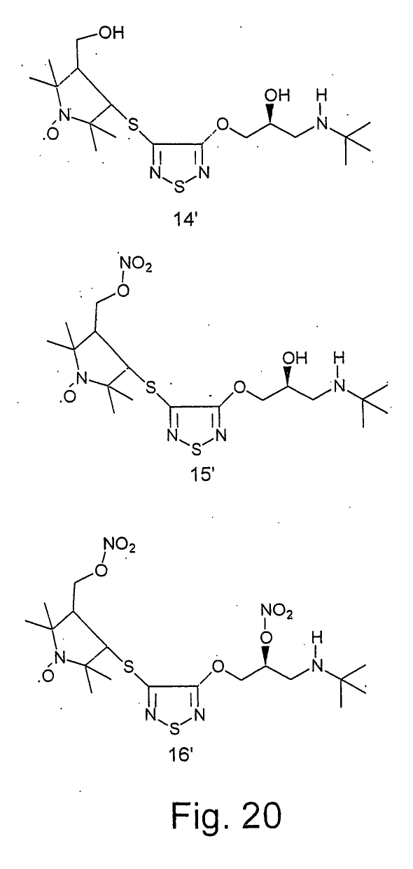

The multifunctional beta-antagonist compounds also include, but are not limited to the multifunctional beta-antagonist compounds of Formulae I, II, and IA, as described herein. In some embodiments of the multifunctional and functionalized beta-antagonist compounds, compositions and methods described herein, include compounds of Formula I (e.g., 14, 15, 20, and 21 as shown in FIG. 4), compounds of Formula II (e.g., 22, 23, 24, 25 as shown in FIG. 5), and compounds of Formula IA (e.g., 1′, 2′, 17′, 18′ 19′ and 20′ as shown in FIG. 16). Exemplary functionalized beta-antagonist compounds may be found in FIGS. 4-20.

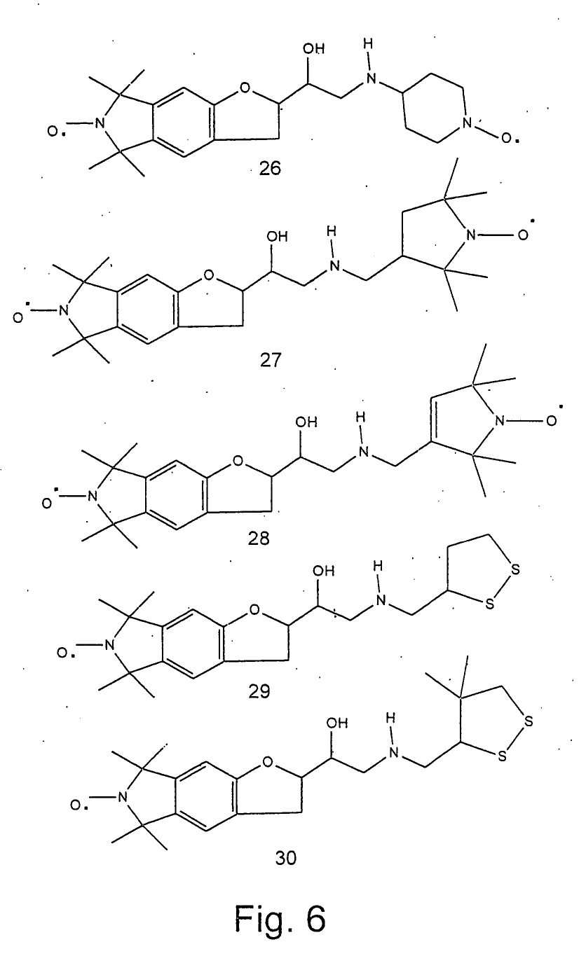

Multifunctional beta-antagonist compounds may be obtained from the exemplary compounds in FIGS. 6-15 by substituting the OH group in compounds 26-75 by an NO donor component or SH. Synthetic methods for these processes are well known to those of skill in the art.

In one embodiment, multifunctional beta-antagonist compounds are provided having Formula I: 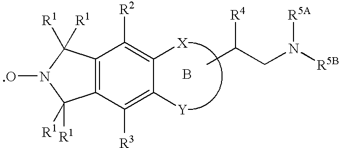
wherein R¹ may be independently hydrogen, substituted or unsubstituted alkyl (e.g., C₁-C₄ alkyl, such as, methyl, ethyl, propyl, isopropyl, tert-butyl), substituted or unsubstituted alkenyl, substituted or unsubstituted alkynyl, substituted or unsubstituted cycloalkyl, substituted or unsubstituted cycloalkenyl, substituted or unsubstituted alkoxy, substituted or unsubstituted aryloxy, substituted or unsubstituted aryl, or substituted or unsubstituted heterocycles,
   wherein the optional substituent may be, for example, a group capable of donating NO in a charged or neutral form NO (e.g., ONO, ONO₂, SNO, N(NO)₂);
R² and R³ may be independently hydrogen, or (CH₂)ₙX¹ (where n is, for example 0, 1, 2, 3, or 4), and X¹ may include, H, OH, ═O (where n is not 0), or a group capable of donating NO (e.g., ONO, ONO₂, SNO, N(NO)₂), or
R² and R³ may be independently H, OH, substituted or unsubstituted alkyl (e.g, C₁-C₄ alkyl, such as, methyl, ethyl, propyl, isopropyl, tert-butyl), substituted or unsubstituted alkenyl, substituted or unsubstituted alkynyl, substituted or unsubstituted cycloalkyl, substituted or unsubstituted cycloalkenyl, substituted or unsubstituted aryl, or substituted or unsubstituted heterocycles,
   wherein the optional substituent may be, for example, a group capable of donating NO in a charged or neutral form NO (e.g., ONO, ONO₂, SNO, N(NO)₂);
R⁴ may be (CH₂)ₘX² (where m is, for example 0, 1, 2, 3, or 4), and X² is H, SH, OH, ═O (where m is not 0), or a group capable of donating NO (e.g., ONO, ONO₂, SNO, N(NO)₂), or
R⁴ may be H, SH, OH, substituted or unsubstituted alkyl (e.g., C₁-C₄ alkyl, such as, methyl, ethyl, propyl, isopropyl, tert-butyl), substituted or unsubstituted alkenyl, substituted or unsubstituted alkynyl, substituted or unsubstituted acyl, substituted or unsubstituted acyloxy, substituted or unsubstituted aminoalkyl, substituted or unsubstituted alkoxy, substituted or unsubstituted aryloxy, substituted or unsubstituted arylamine, substituted or unsubstituted arylsulphide, substituted or unsubstituted arylsulphone, substituted or unsubstituted arylsulfurdioxide, substituted or unsubstituted aryl, substituted or unsubstituted aryloxy, or substituted or unsubstituted heterocycles,
   wherein the optional substituent may be, for example, a group capable of donating NO in a charged or neutral form NO (e.g., ONO, ONO₂, SNO, N(NO)₂);
R^{5A} and R^{5B} may be, independently, (CH₂)ₚ X³ (where p is, for example 0, 1, 2, 3, or 4), and X³ is H, OH, ═O (where p is not 0), or a group capable of donating NO (e.g., ONO, ONO₂, SNO, N(NO)₂), or
R^{5A} and R^{5B} may be, independently, H, OH, ═O, substituted or unsubstituted alkyl (e.g, C₁-C₄ alkyl, such as, methyl, ethyl, propyl, isopropyl, tert-butyl), substituted or unsubstituted alkenyl, substituted or unsubstituted alkynyl, substituted or unsubstituted acyl, substituted or unsubstituted acyloxy, substituted or unsubstituted aminoalkyl, substituted or unsubstituted alkoxy, substituted or unsubstituted arylamine, substituted or unsubstituted arylsulphide, substituted or unsubstituted arylsulphone, substituted or unsubstituted arylsulfurdioxide, substituted or unsubstituted aryl, substituted or unsubstituted aryloxy, or substituted or unsubstituted heterocycles,
   wherein the optional substituent may be, for example, a group capable of donating NO in a charged or neutral form NO (e.g., ONO, ONO₂, SNO, N(NO)₂); and
X and Y may independently be —CH═CH—, (CH₂)_{q} (where q is, for example 0, 1, 2, or 3), O, S, NH, CH₂, or NR⁷,
   wherein R⁷ may be hydrogen, substituted or unsubstituted alkyl (e.g, C₁-C₄ alkyl, such as, methyl, ethyl, propyl, isopropyl, tert-butyl), substituted or unsubstituted alkenyl, substituted or unsubstituted alkynyl, substituted or unsubstituted acyl, substituted or unsubstituted acyloxy, substituted or unsubstituted aminoalkyl, substituted or unsubstituted alkoxy, substituted or unsubstituted arylamine, substituted or unsubstituted arylsulphide, substituted or unsubstituted arylsulphone, substituted or unsubstituted arylsulfurdioxide, substituted or unsubstituted aryl, substituted or unsubstituted aryloxy, substituted or unsubstituted heterocycles;
   wherein the optional substituent may be, for example, a group capable of donating NO in a charged or neutral form NO (e.g., ONO, ONO₂, SNO, N(NO)₂); and
where ring B is independently a 5-, 6- or 7-membered ring.

In some embodiments of the invention, R^{5A} and R^{5B} in Formula I are substituted with a substituent comprising a group capable of donating NO (e.g., ONO, ONO₂, SNO, N(NO)₂). In certain embodiments, the NO donor group may be ONO, ONO₂, or SNO.

In some embodiments of Formula I, R^{5A} and/or R^{5A} comprises a substituted pyridinyloxy free radical.

In certain embodiments of the invention R¹ may be independently hydrogen, substituted or unsubstituted alkyl (e.g., C₁-C₄ alkyl, such as, methyl, ethyl, propyl, isopropyl, tert-butyl);
R² and R³ may be independently hydrogen, H, SH, OH, NH; or
R⁴ may be independently H, SH, OH, NH, or a group capable of donating NO (e.g., ONO, ONO₂, SNO, N(NO)₂); or
R^{5A} and R^{5B} may be, independently, H or substituted or unsubstituted alkyl (e.g., C₁-C₄ alkyl, such as, methyl, ethyl, propyl, isopropyl, tert-butyl);
   wherein the alkyl group may be substituted with SH or a group capable of donating NO (e.g., ONO, ONO₂, SNO, N(NO)₂); and
X and Y may independently be —CH═CH—, (CH₂)_{q} (where q is, for example 0, 1, 2, or 3), O, S, substituted or unsubstituted alkyl (e.g, C₁-C₄ alkyl, such as, methyl, ethyl, propyl, isopropyl, tert-butyl), or NR⁷,
   wherein R⁷ may be hydrogen, substituted or unsubstituted alkyl (e.g., C₁-C₄ alkyl, such as, methyl, ethyl, propyl, isopropyl, tert-butyl);
where ring B is independently a 5-, 6- or 7-membered ring, and
where substituted or unsubstituted substituents may be substituted with, for example, an NO donor group (e.g., ONO, SNO or ONO₂, etc.).

In other embodiments of the invention, R¹ in Formula I may be independently hydrogen, substituted or unsubstituted alkyl (e.g., C₁-C₄ alkyl, such as, methyl, ethyl, propyl, isopropyl, tert-butyl);

R² and R³ may be independently hydrogen, or (CH₂)ₙX¹ (where n is, for example 0, 1, 2, 3, or 4), and X¹ may include, H, OH, ═O (where n is not 0), or a group capable of donating NO (e.g., ONO, ONO₂, SNO, N(NO)₂), or
R² and R³ may be independently, H, OH, substituted or unsubstituted alkyl (e.g, C₁-C₄ alkyl, such as, methyl, ethyl, propyl, isopropyl, tert-butyl);
R⁴ may be (CH₂)ₘX² (where m is, for example 0, 1, 2, 3, or 4), and X² is SH, OH, ═O (where n is not 0), or a group capable of donating NO (e.g., ONO, ONO₂, SNO, N(NO)₂), or
R⁴ may be SH, OH, substituted or unsubstituted alkyl (e.g, C₁-C₄ alkyl, such as, methyl, ethyl, propyl, isopropyl, tert-butyl);
R^{5A} and R^{5B} may be, independently, (CH₂)ₚ X³ (where p is, for example 0, 1, 2, 3, or 4), and X³ is H, OH, ═O (where n is not 0), or a group capable of donating NO (e.g., ONO, ONO₂, SNO, N(NO)₂), or
R^{5A} and R^{5B} may be, independently, H, OH, substituted or unsubstituted alkyl (e.g, C₁-C₄ alkyl, such as, methyl, ethyl, propyl, isopropyl, tert-butyl); and
X and Y may independently be —CH═CH—, (CH₂)_{q} (where q is, for example 0, 1, 2, or 3), O, S, substituted or unsubstituted alkyl (e.g., C₁-C₄ alkyl, such as, methyl, ethyl, propyl, isopropyl, tert-butyl) or NR⁷,
   wherein R⁷ may be hydrogen, substituted or unsubstituted alkyl (e.g, C₁-C₄ alkyl, such as, methyl, ethyl, propyl, isopropyl, tert-butyl);
where ring B is independently a 5-, 6- or 7-membered ring, and
where substituted substituents may be substituted with, for example, an NO donor group (e.g., ONO, SNO or ONO₂, N(NO)₂).

In other preferred embodiments, R¹ may be independently hydrogen, substituted or unsubstituted alkyl (e.g., C₁-C₄ alkyl, such as, methyl, ethyl, propyl, isopropyl, tert-butyl);
R² and R³ may be independently hydrogen, or (CH₂)ₙX¹ (where n is, for example 0, 1, 2, 3, or 4), and X¹ may include, H, OH, or a group capable of donating NO (e.g., ONO, ONO₂, SNO, N(NO)₂), or
R² and R³ may be independently H, OH, substituted or unsubstituted alkyl (e.g., C₁-C₄ alkyl, such as, methyl, ethyl, propyl, isopropyl, tert-butyl);
R⁴ may be (CH₂)ₘX² (where m is, for example 0, 1, 2, 3, or 4), and X² is H, OH, SH or a group capable of donating NO (e.g., ONO, ONO₂, SNO, N(NO)₂), or
R⁴ may be SH, OH, substituted or unsubstituted alkyl (e.g., C₁-C₄ alkyl, such as, methyl, ethyl, propyl, isopropyl, tert-butyl);
R^{5A} and R^{5B} may be, independently, (CH₂)ₚ X³ (where p is, for example 0, 1, 2, 3, or 4), and X³ is H, OH, or a group capable of donating NO (e.g., ONO, ONO₂, SNO, N(NO)₂), or
R^{5A} and R^{5B} may be, independently, H, OH, substituted or unsubstituted alkyl (e.g., C₁-C₄ alkyl, such as, methyl, ethyl, propyl, isopropyl, tert-butyl); and
X and Y may independently be —CH═CH—, (CH₂)_{q} (where q is, for example 0, 1, 2, or 3), S, O, substituted or unsubstituted alkyl (e.g, C₁-C₄ alkyl, such as, methyl, ethyl, propyl, isopropyl, tert-butyl) or NR⁷,
   wherein R⁷ may be hydrogen, substituted or unsubstituted alkyl (e.g., C₁-C₄ alkyl, such as, methyl, ethyl, propyl, isopropyl, tert-butyl);
where ring B is independently a 5-, 6- or 7-membered ring, and
where substituted or unsubstituted substituents may be substituted with, for example, an NO donor group (e.g., ONO, SNO or ONO₂, etc.).

In preferred embodiments of the invention, the multifunctional beta-antagonist compounds have structures 14, 15, 20, and 21, as shown in Schemes 1 and 2 and in FIG. 4.

In other embodiments of this invention, multifunctional beta-antagonist compounds of are provided, having formula II: 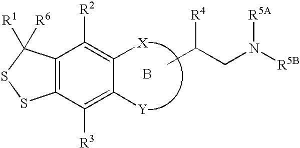
wherein R¹ and R⁶ may be independently hydrogen, substituted or unsubstituted alkyl (e.g., C₁-C₄ alkyl, such as, methyl, ethyl, propyl, isopropyl, tert-butyl), substituted or unsubstituted alkenyl, substituted or unsubstituted alkynyl, substituted or unsubstituted cycloalkyl, substituted or unsubstituted cycloalkenyl, substituted or unsubstituted alkoxy, substituted or unsubstituted aryloxy, substituted or unsubstituted aryl, or substituted or unsubstituted heterocycles,
   wherein the optional substituent may be, for example, a group capable of donating NO in a charged or neutral form NO (e.g., ONO, ONO₂, SNO, N(NO)₂);
R² and R³ may be independently hydrogen, or (CH₂)ₙX¹ (where n is, for example 0, 1, 2, 3, or 4), and X¹ may include, H, OH, ═O (where n is not 0), or a group capable of donating NO (e.g., ONO, ONO₂, SNO, N(NO)₂), or
R² and R³ may be independently H, OH, substituted or unsubstituted alkyl (e.g, C₁-C₄ alkyl, such as, methyl, ethyl, propyl, isopropyl, tert-butyl), substituted or unsubstituted alkenyl, substituted or unsubstituted alkynyl, substituted or unsubstituted cycloalkyl, substituted or unsubstituted cycloalkenyl, substituted or unsubstituted alkoxy, substituted or unsubstituted aryloxy, substituted or unsubstituted aryl, or substituted or unsubstituted heterocycles,
   wherein the optional substituent may be, for example, a group capable of donating NO in a charged or neutral form NO (e.g., ONO, ONO₂, SNO, N(NO)₂);
R⁴ may be (CH₂)ₘX² (where m is, for example 0, 1, 2, 3, or 4), and X² is H, SH, OH, ═O (where m is not 0), or a group capable of donating NO (e.g., ONO, ONO₂, SNO, N(NO)₂), or
R⁴ may be H, SH, OH, substituted or unsubstituted alkyl (e.g, C₁-C₄ alkyl, such as, methyl, ethyl, propyl, isopropyl, tert-butyl), substituted or unsubstituted alkenyl, substituted or unsubstituted alkynyl, substituted or unsubstituted acyl, substituted or unsubstituted acyloxy, substituted or unsubstituted aminoalkyl, substituted or unsubstituted alkoxy, substituted or unsubstituted arylamine, substituted or unsubstituted arylsulphide, substituted or unsubstituted arylsulphone, substituted or unsubstituted arylsulfurdioxide, substituted or unsubstituted aryl, substituted or unsubstituted aryloxy, or substituted or unsubstituted heterocycles,
   wherein the optional substituent may be, for example, a group capable of donating NO in a charged or neutral form NO (e.g., ONO, ONO₂, SNO, N(NO)₂);
R^{5A} and R^{5B} may be, independently, (CH₂)ₚ X³ (where p is, for example 0, 1, 2, 3, or 4), and X³ is H, OH, ═O (where p is not 0), or a group capable of donating NO (e.g., ONO, ONO₂, SNO, N(NO)₂), or R^{5A} and R^{5B} may be, independently, H, OH, ═O, substituted or unsubstituted alkyl (e.g, C₁-C₄ alkyl, such as, methyl, ethyl, propyl, isopropyl, tert-butyl), substituted or unsubstituted alkenyl, substituted or unsubstituted alkynyl, substituted or unsubstituted acyl, substituted or unsubstituted acyloxy, substituted or unsubstituted aminoalkyl, substituted or unsubstituted alkoxy, substituted or unsubstituted arylamine, substituted or unsubstituted arylsulphide, substituted or unsubstituted arylsulphone, substituted or unsubstituted arylsulfurdioxide, substituted or unsubstituted aryl, substituted or unsubstituted aryloxy, or substituted or unsubstituted heterocycles,
   wherein the optional substituent may be, for example, a group capable of donating NO in a charged or neutral form NO (e.g., ONO, ONO₂, SNO, N(NO)₂); and
X and Y may independently be —CH═CH—, (CH₂)_{q} (where q is, for example 0, 1, 2, or 3), O, S, NH, CH₂, or NR⁷,
   wherein R⁷ may be hydrogen, substituted or unsubstituted alkyl (e.g., C₁-C₄ alkyl, such as, methyl, ethyl, propyl, isopropyl, tert-butyl), substituted or unsubstituted alkenyl, substituted or unsubstituted alkynyl, substituted or unsubstituted acyl, substituted or unsubstituted acyloxy, substituted or unsubstituted aminoalkyl, substituted or unsubstituted alkoxy, substituted or unsubstituted arylamine, substituted or unsubstituted arylsulphide, substituted or unsubstituted arylsulphone, substituted or unsubstituted arylsulfurdioxide, substituted or unsubstituted aryl, substituted or unsubstituted aryloxy, substituted or unsubstituted heterocycles;
   wherein the optional substituent may be, for example, a group capable of donating NO in a charged or neutral form NO (e.g., ONO, ONO₂, SNO, N(NO)₂); and
where ring B is independently a 5-, 6- or 7-membered ring.

In Formula II, R^{5A} and R^{5B} are preferably substituted with a substituent comprising a group capable of donating NO (e.g., ONO, ONO₂, SNO, N(NO)₂). In certain embodiments, the NO donor group may be ONO, ONO₂, or SNO.

In some embodiments, R^{5A} and/or R^{5A} comprises a substituted pyridinylyloxy free radical.

In other embodiments, R^{5A} and/or R^{5A} in Formula II comprises a substituted pyridinylyloxy free radical.

In still further embodiments of this invention, R¹ and R⁶ in Formula II may be independently hydrogen, substituted or unsubstituted alkyl (e.g., C₁-C₄ alkyl, such as, methyl, ethyl, propyl, isopropyl, tert-butyl);
R² and R³ may be independently hydrogen, H, SH, OH, NH; or
R⁴ may be independently H, SH, OH, NH, or a group capable of donating NO (e.g., ONO, ONO₂, SNO, N(NO)₂); or
R^{5A} and R^{5B} may be, independently, H or substituted or unsubstituted alkyl (e.g., C₁-C₄ alkyl, such as, methyl, ethyl, propyl, isopropyl, tert-butyl);
   wherein the alkyl group may be substituted with SH or a group capable of donating NO (e.g., ONO, ONO₂, SNO, N(NO)₂); and
X and Y may independently be —CH—CH—, (CH₂)_{q} (where q is, for example 0, 1, 2, or 3), O, S, substituted or unsubstituted alkyl (e.g, C₁-C₄ alkyl, such as, methyl, ethyl, propyl, isopropyl, tert-butyl), or NR⁷,
   wherein R⁷ may be hydrogen, substituted or unsubstituted alkyl (e.g., C₁-C₄ alkyl, such as, methyl, ethyl, propyl, isopropyl, tert-butyl);
where ring B is independently a 5-, 6- or 7-membered ring, and
where substituted or unsubstituted substituents may be substituted with, for example, an NO donor group (e.g., ONO, SNO or ONO₂, etc.).
R¹ in Formula I is preferably hydrogen, substituted or unsubstituted alkyl (e.g, C₁-C₄ alkyl, such as, methyl, ethyl, propyl, isopropyl, tert-butyl);
R² and R³ may be independently hydrogen, or (CH₂)ₙX¹ (where n is, for example 0, 1, 2, 3, or 4), and X¹ may include, H, OH, ═O, or a group capable of donating NO (e.g., ONO, ONO₂, SNO, N(NO)₂), or
R² and R³ may be independently, H, OH, substituted or unsubstituted alkyl (e.g., C₁-C₄ alkyl, such as, methyl, ethyl, propyl, isopropyl, tert-butyl);
R⁴ may be (CH₂)ₘX² (where m is, for example 0, 1, 2, 3, or 4), and X² is SH, OH, ═O, or a group capable of donating NO (e.g., ONO, ONO₂, SNO, N(NO)₂), or
R⁴ may be SH, OH, substituted or unsubstituted alkyl (e.g., C₁-C₄ alkyl, such as, methyl, ethyl, propyl, isopropyl, tert-butyl);
R^{5A} and R^{5B} may be, independently, (CH₂)ₚ X³ (where p is, for example 0, 1, 2, 3, or 4), and X³ is H, OH, ═O (where n is not 0), or a group capable of donating NO (e.g., ONO, ONO₂, SNO, N(NO)₂); or
R^{5A} and R^{5B} may be, independently, H, OH, substituted or unsubstituted alkyl (e.g, C₁-C₄ alkyl, such as, methyl, ethyl, propyl, isopropyl, tert-butyl); and
X and Y may independently be —CH═CH—, (CH₂)_{q} (where q is, for example 0, 1, 2, or 3), O, S, substituted or unsubstituted alkyl (e.g., C₁-C₄ alkyl, such as, methyl, ethyl, propyl, isopropyl, tert-butyl) or NR⁷,
   wherein R⁷ may be hydrogen, substituted or unsubstituted alkyl (e.g, C₁-C₄ alkyl, such as, methyl, ethyl, propyl, isopropyl, tert-butyl);
where ring B is independently a 5-, 6- or 7-membered ring, and
where substituted substituents may be substituted with, for example, an NO donor group (e.g., ONO, SNO or ONO₂, N(NO)₂).

In certain embodiments, in Formula II,
R¹ may be independently hydrogen, substituted or unsubstituted alkyl (e.g., C₁-C₄ alkyl, such as, methyl, ethyl, propyl, isopropyl, tert-butyl);
R² and R³ may be independently hydrogen, or (CH₂)ₙX¹ (where n is, for example 0, 1, 2, 3, or 4), and X¹ may include, H, OH, or a group capable of donating NO (e.g., ONO, ONO₂, SNO, N(NO)₂), or
R² and R³ may be independently H, OH, substituted or unsubstituted alkyl (e.g., C₁-C₄ alkyl, such as, methyl, ethyl, propyl, isopropyl, tert-butyl);
R⁴ may be (CH₂)ₘX² (where m is, for example 0, 1, 2, 3, or 4), and X² is H, OH, SH or a group capable of donating NO (e.g., ONO, ONO₂, SNO, N(NO)₂); or
R⁴ may be SH, OH, substituted or unsubstituted alkyl (e.g., C₁-C₄ alkyl, such as, methyl, ethyl, propyl, isopropyl, tert-butyl);
R^{5A} and R^{5B} may be, independently, (CH₂)ₚ X³ (where p is, for example 0, 1, 2, 3, or 4), and X³ is H, OH, or a group capable of donating NO (e.g., ONO, ONO₂, SNO, N(NO)₂), or
R^{5A} and R^{5B} may be, independently, H, OH, substituted or unsubstituted alkyl (e.g., C₁-C₄ alkyl, such as, methyl, ethyl, propyl, isopropyl, tert-butyl); and
X and Y may independently be —CH═CH—, (CH₂)_{q} (where q is, for example 0, 1, 2, or 3), S, O, substituted or unsubstituted alkyl (e.g., C₁-C₄ alkyl, such as, methyl, ethyl, propyl, isopropyl, tert-butyl) or NR⁷,
   wherein R⁷ may be hydrogen, substituted or unsubstituted alkyl (e.g., C₁-C₄ alkyl, such as, methyl, ethyl, propyl, isopropyl, tert-butyl);
where ring B is independently a 5-, 6- or 7-membered ring, and
where substituted or unsubstituted substituents may be substituted with, for example, an NO donor group (e.g., ONO, SNO or ONO₂, etc.).

In preferred embodiments of this invention, R^{5A} and R^{5B} in Formulae I and II may independently be selected from the groups listed in Tables I-IV, below.

TABLE I

R^{5A}/R^{5B}
CH₃—
C₅H₉—
C₆H₅SO₂O—
CH₃CO—
C₆H₅SO₂NH—
(C₆H₅SO₂)₂N—
C₄H₈
C₅H₁₀
C₅H₁₁

TABLE II

| R^{5A} | R^{5B} |
|---|---|
| CH₃— | —C(CH₃)₃ |
| (CH₃)₂CH— | —C(CH₃)₃ |
| CH₃CH₂CH₂— | —C(CH₃)₃ |
| CH₃— | —CH(CH₃)2 |
| CH₃— | —C(CH₃)₂CH₂OH |
| CH₃CH₂CH₂CH₂— | —C(CH₃)₃ |
| CH₃— | 4-CF₃—Ph— |
| CH₃CH₂— | —C(CH₃)₃ |
| CH₃— | —CH₃ |
| CH₃— | 3,4,5-tri(CH₃O—)Ph— |

TABLE III

| R^{5A} | R^{5B} |
|---|---|
| CH₃CH₂— | —C(CH₃)₃ |
| CH₃CH₂CH₂— | —C(CH₃)₃ |
| CH₃CH₂CH₂CH₂— | —C(CH₃)₃ |
| CH₃CH₂OC(O)CH₂CH₂— | —C(CH₃)₃ |
| CH₃CH₂OC(O)CH₂— | —C(CH₃)₃ |

TABLE IV

| R^{5A} | R^{5B} |
|---|---|
| CH₃— | —C(CH₃)₃ |
| CH₃—O—CH₂CH₂— | —C(CH₃)₃ |
| CH₃— | —CH₂CH₂CH(SCH₃)CH₃ |

In certain embodiments the multifunctional beta-antagonists are 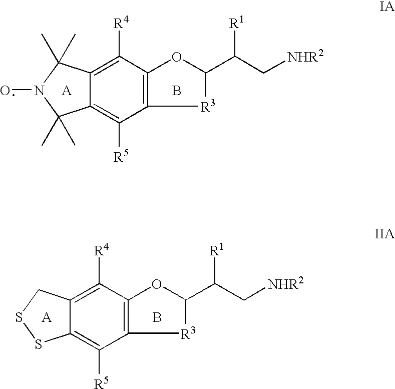
R¹═OH, ONO, ONO₂, SH, SNO
R²=Straight/branched/functionalized alkyl chain C₃-C₁₀
R³═CH₂, CH₂—CH₂, NH, S, CH₂S, O, CH₂O
R⁴═H, Straight/branched/functionalized alkyl chain C₁-C₈
R⁵═H, Straight/branched/functionalized alkyl chain C₁-C₈
A=5, 6, or 7-membered ring
B=5, 6, or 7-membered ring

In other embodiments, the multifunctional antagonists are compounds according to 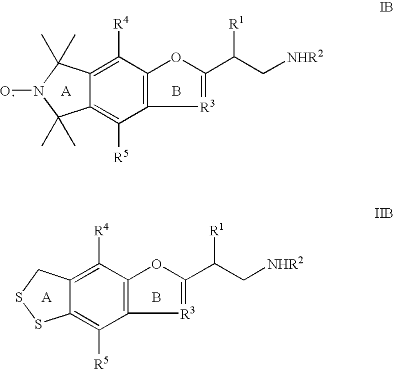
R¹═OH, ONO, ONO₂, SH, SNO
R²=Straight/branched/functionalized alkyl chain C₃-C₁₀
R³═CH, CH₂CH, N, SCH₂
R⁴═H, Straight/branched/functionalized allyl chain C₁-C₈
R⁵═H, Straight/branched/functionalized alkyl chain C₁-C₈
A=5, 6, or 7-membered ring
B=5, 6, or 7-membered ring

In other embodiment of this invention, compounds of Formula IA are provided: 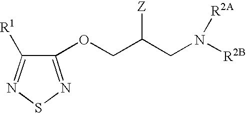
wherein R¹ is a group comprising a substituted N-oxide free radical, wherein the N-oxide free radical is contained within a 5- or 6-membered ring, and optionally further comprises a group capable of donating, or R¹ is a group selected from IIA, IIIA, IVA, and VA as shown below, where the groups of Formulae IIA-VA are linked to Formula IA at position R¹, through substituent Y of Formulae IIA-VA;
Z is halo, nitrato, nitroso, nitrile, hydroxyl, thiol, sulfonamido, amino, guanadino, isoguanadino, cyano, isocyano, and carboxyl; and

R^{2A} and R^{2B} are independently hydrogen, ═O, alkyl, substituted alkyl, alkenyl, substituted alkenyl, alkynyl, substituted alkynyl, cycloalkyl, substituted cycloalkyl, cycloalkenyl, substituted cycloalkenyl, phenyl, substituted phenyl or heterocyclyl, wherein the optional substitution comprises one or more groups capable of donating NO group, or one or more groups being ROS scavengers; and wherein
IIA has structure 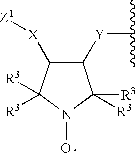
in which R³ is independently hydrogen or C₁-C₄ alkyl;
   Y is selected from (CH₂)ₙ while n being from 0 to 3, O, NH, S, substituted or unsubstituted acyl, acyloxy, alkynyl, alkene, alkyl, alkoxy, aryloxy, arylamine, arylsulphide, arylsulphone, or arylsulfurdioxide; X is selected from (CHₘ)ₚ while m being 2 or 3 and p from 0 to 3, O, —N═N—, S, NH, CH₃N—, substituted or unsubstituted acyl, acyloxy, alkynyl, alkene, alkyl, alkoxy, aryloxy, arylamine, arylsulphide, arylsulphone, arylsulfurdioxide; and Z¹ is H OH, ONO, ONO₂, SNO;
IIIA has structure 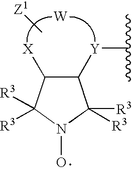
in which R³ is independently hydrogen or C₁-C₄ alkyl;
   W is is (CHₘ)ₙ while m being from 0 to 2 and n being from 0 to 3, where W may be substituted or unsubstituted; X is selected from (CHₚ) while p 1 or 2, O, —N═N—, S, and NH, or X is absent; Y is (CH) or N; and Z¹ is selected from H, OH, CH₂ONO, CH₂CH₂ONO, CH₂ONO₂, CH₂CH₂ONO₂, ONO, ONO₂, SNO, and NONOate; wherein IIIA may include 1-3 Z¹ substituents on the ring to which Z¹ is attached;
IVA has structure 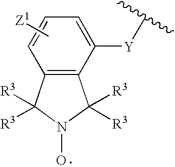
in which R³ may be independently hydrogen or C₁-C₄ alkyl; Y may be CH₂, O, NH, S, substituted or unsubstituted alkylene, or it can be absent; Z¹ may be CH₂ONO, CH₂CH₂ONO, CH₂ONO₂, CH₂CH₂ONO₂, NO, NO₂, ONO, ONO₂, SNO, or OH, wherein IVA may include 1-3 of the Z¹ substituents on the ring to which Z¹ is attached; 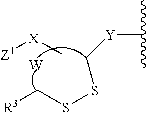
VA has structure
   in which R³ is hydrogen or C₁-C₄ alkyl; W is (CHₘ)ₙ while m being from 0 to 2 and n being from 0 to 3, where W can be substituted or unsubstituted; X is selected from (CHₚ) while p being 1 or 2, O, —N═N—, S, NH, CH₃N—, and substituted or unsubstituted alkylene, or X is absent; Y is selected from CH₂, O, NH, S or substituted or unsubstituted alkylene, or Y is absent; and Z¹ is OH, ONO, ONO₂, N(NO)₂, or SNO.

In preferred embodiments, R^{2A} and R^{2B} in compound IIA are selected as shown in Tables I, II, III, and IV

TABLE I

R^{2A}/R^{2B}
CH₃—
C₅H₉—
C₆H₅SO₂O—
CH₃CO—
C₆H₅SO₂NH—
(C₆H₅SO₂)₂N—
C₄H₈
C₅H₁₀
C₅H₁₁

TABLE II

| R^{2A} | R^{2B} |
|---|---|
| CH₃— | —C(CH₃)₃ |
| (CH₃)₂CH— | —C(CH₃)₃ |
| CH₃CH₂CH₂— | —C(CH₃)₃ |
| CH₃— | —CH(CH₃)₂ |
| CH₃— | —C(CH₃)₂CH₂OH |
| CH₃CH₂CH₂CH₂— | —C(CH₃)₃ |
| CH₃— | 4-CF₃—Ph— |
| CH₃CH₂— | —C(CH₃)₃ |
| CH₃— | —CH₃ |
| CH₃— | 3,4,5-tri(CH₃O—)Ph— |

TABLE III

| R^{2A} | R^{2B} |
|---|---|
| CH₃CH₂— | —C(CH₃)₃ |
| CH₃CH₂CH₂— | —C(CH₃)₃ |
| CH₃CH₂CH₂CH₂— | —C(CH₃)₃ |
| CH₃CH₂OC(O)CH₂CH₂— | —C(CH₃)₃ |
| CH₃CH₂OC(O)CH₂— | —C(CH₃)₃ |

TABLE IV

| R^{2A} | R^{2B} |
|---|---|
| CH₃— | —C(CH₃)₃ |
| CH₃—O—CH₂CH₂— | —C(CH₃)₃ |
| CH₃— | —CH₂CH₂CH(SCH₃)CH₃ |

Many preferred structures of Formulae I, II, and IA have Formula III 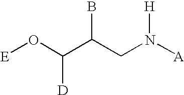
wherein A is C₁-C₄ alkyl or ROS-scavenger group;
B is selected from OH, O—NO₂ and SH;
D is H, or D is (CH₂)₂ and is connected to E and together with the neighboring atoms forms a 5-6 membered ring consisting of carbon atoms and one oxygen atom; and
E is phenyl condensed with optionally substituted phenyl or optionally substituted 5-6 membered heterocycle containing one of —N—, —O—, and —S—S—; or
E is thiadiazolyl substituted with morpholinyl or pyrrolidinyl-N-oxide, said morpholinyl being optionally substituted with one of OH, NO-donor group, and ROS-scavenger group, and said pyrrolidinyl-N-oxide group being bound to said thiadiazolyl vial —S— or via —CH₂—O—.
   Synthesis of Multifunctional β-Antagonist Compounds

Multifunctional β-antagonist compounds may be synthesized as described herein using methods available in the art and standard techniques in organic chemistry, as described, for example, in March's Advanced Organic Chemistry: Reactions, Mechanisms, and Structure, 5th Edition (2000) M. B. Smith & J. March, John Wiley & Sons, New York, N.Y.; Organic Chemistry 6^{th} Ed. (1992) R. Morrison & R Boyd, Benjamin Cummings, San Francisco; and Richard C. Larock, “Comprehensive Organic Transformations” New York: Wiley-VCH; 1989.

The multifunctional beta-antagonists of this invention can be prepared from readily available starting materials using the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

The general approach for synthesis of compounds of the general Formula I is outlined in Scheme 1 below:

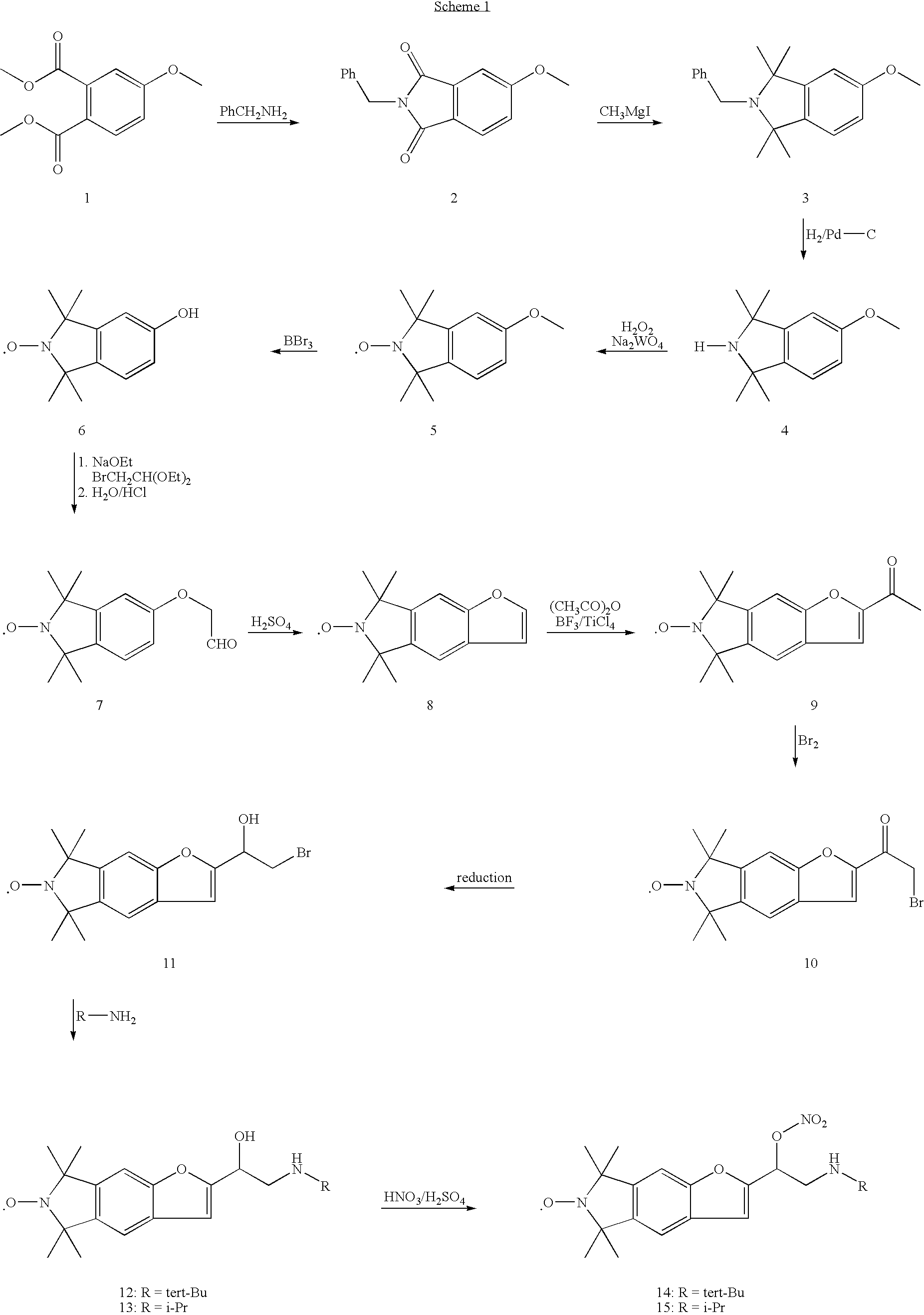

According to Scheme 1 above, multifunctional beta-antagonist compounds according to Formula I may be synthesized as follows. The commercially available starting material, dimethyl 4-methoxyphthalate (1), is treated with equivalent amount of benzylamine to afford 2-benzyl-5-methoxyphthalimide (2) that may react with excess methylmagnesium iodide to provide 2-benzyl-5-methoxy-1,1,3,3-tetramethylisoindoline (3). Catalytic hydrogenation of this compound gives the corresponding debenzylated product 5-methoxy-1,1,3,3-tetramethylisoindoline (4) that is oxidized with hydrogen peroxide and sodium tungstate to generate the nitroxide radical 5-methoxy-2-oxy-1,1,3,3-tetramethylisoindoline (5). Treatment of this compound with boron tribromide provides demethylated intermediate 5-hydroxy-2-oxy-1,1,3,3-tetramethylisoindoline (6) that may react with bromoacetaldehyde diethyl acetal in the presence of a base such as sodium ethoxide, then hydrolyzed in acidic conditions such as dilute hydrochloric acid to generate the alkylated alhehyde 5-(formylmethoxy)-2-oxy-1,1,3,3-tetramethylisoindoline (7). This aldehyde is cyclized by treatment with strong dehydrating agents such as sulfuric acid to give the nitroxide benzofuran compound 5,5,7,7-tetramethyl-6-oxy-6,7-dihydro-5H-1-oxa-6-aza-s-indacene (8) that is acetylated with acetic anhydride in the presence of BF₃/TiCl₄ to afford compound 1-(5,5,7,7-tetramethyl-6-oxy-6,7-dihydro-5H-1-oxa-6-aza-s-indacen-2-yl)ethanone (9). Bromination of this intermediate with bromine generates the bromoacetyl benzofuran nitroxide 2-bromo-1-(5,5,7,7-tetramethyl-6-oxy-6,7-dihydro-5H-1-oxa-6-aza-s-indacen-2-yl)-ethanone (10), which gives the alcohol-2-bromo-1-(5,5,7,7-tetramethyl-6-oxy-6,7-dihydro-5H-1-oxa-6-aza-s-indacen-2-yl)ethanol (11) by reduction. This bromo ethanol reacts with tert-butylamine or isopropylamine to provide compound 2-tert-butylamino-1-(5,5,7,7-tetramethyl-6-oxy-6,7-dihydro-5H-1-oxa-6-aza-s-indacen-2-yl)ethanol (12) or 2-isopropylamino-1-(5,5,7,7-tetramethyl-6-oxy-6,7-dihydro-5H-1-oxa-6-aza-s-indacen-2-yl)ethanol (13), respectively, which is treated under nitration conditions such as HNO₃/H₂SO₄ or benzoyl nitrate to generate the corresponding product 2-tert-butylamino-1-(5,5,7,7-tetramethyl-6-oxy-6,7-dihydro-5H-1-oxa-6-aza-s-indacen-2-yl)ethyl nitrate (14) or 2-isopropylamino-1-(5,5,7,7-tetramethyl-6-oxy-6,7-dihydro-5H-1-oxa-6-aza-s-indacen-2-yl)ethyl nitrate (15).

A further approach for synthesis of multifunctional beta-antagonist compounds of the Formula I is outlined in Scheme 2 below:

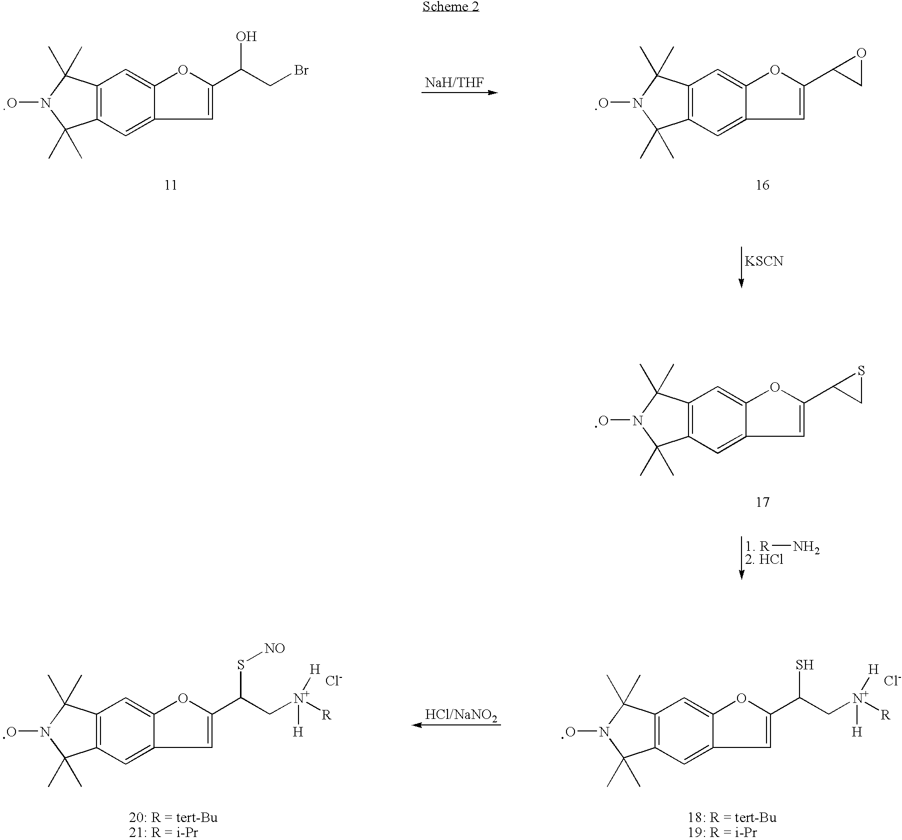

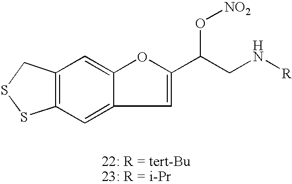

Compounds in Formula II, 22 and 23 are prepared by the similar procedures as described in Scheme 1.

As for compounds of Formula IA, as shown in Scheme I′, the commercially available starting materials 3,4-dichloro-1,2,5-thiadiazole 6 and (S)-(−)3-tert-butylamino-1,2-propanediol 5 may be treated with base such as potassium tert-butoxide in an anhydrous solvent such as tert-butanol to afford the coupling intermediate 3-(3-tert-butylamino-2-hydroxypropyloxy)-4-chloro-1,2,5-thiadiazole 4. This intermediate is then reacted with 3-hydroxymethyl-2,2,5,5-tetramethylpyrrolidinyl-1-oxy 3 in the presence of a base such as potassium tert-butoxide in an anhydrous solvent such as tert-butanol to provide 3-(3-tert-butylamino-2-hydroxypropyloxy)-4-[(2,2,5,5-tetramethyl-1-oxy-3-pyrrolidinyl)methoxy]-1,2,5-thiadiazole 2 that is nitrated with nitration methods such as HNO₃/H₂SO₄ to generate the final compound 1, as shown below in Scheme I′.

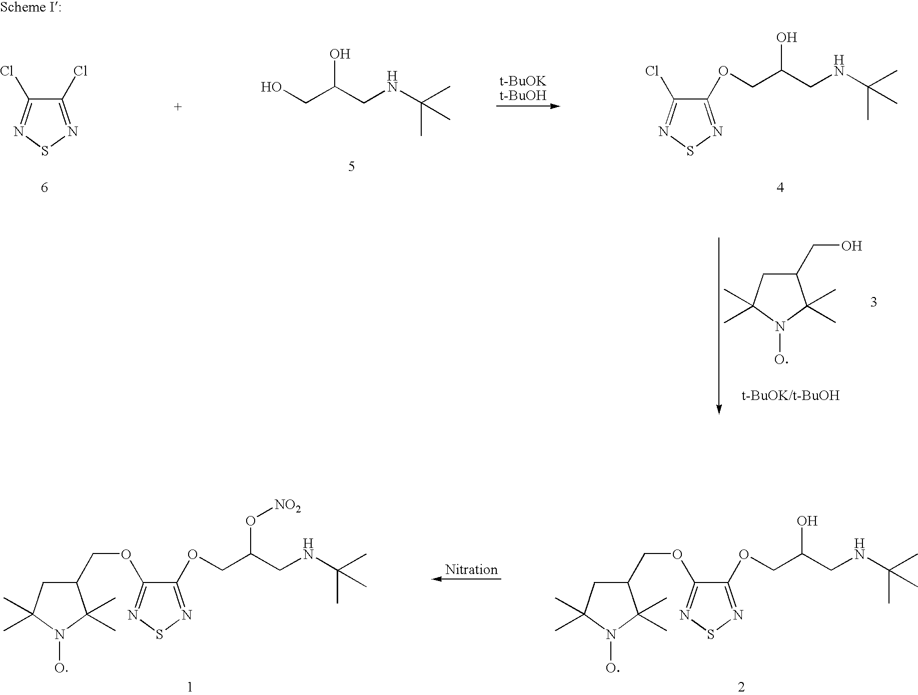

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in T. W. Greene and G. M. Wuts, Protecting Groups in Organic Synthesis, Second Edition, Wiley, New York 1991, and references cited therein.

In a preferred method of synthesis, the multifunctional beta-antagonist compounds of this invention are prepared by coupling an aryl halide compound of formula 5:

Exemplary multifunctional β-antagonist compounds and functionalized β-antagonist compounds are described below and shown in FIGS. 16-20. The details of the syntheses of these multifunctional beta-antagonist compounds and functionalized β-antagonist compounds are described in Examples 5-8.

In certain embodiments of Formula IA, are multifunctional beta-antagonist compounds and functionalized beta-antagonist compounds as shown in figures and as shown hereinbelow:
7. (S)-(−)-3-(3-tert-butylamino-2-hydroxypropoxy)-4-[(2,2,5,5-tetramethyl-1-oxy-3-pyrrolidinyl)methoxy]-1,2,5-thiadiazole 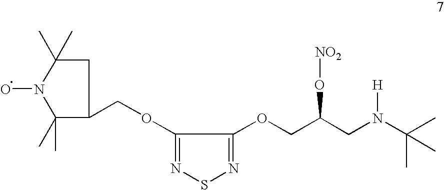
8. (S)-(−)-3-(3-tert-butylamino-2-nitratopropoxy)-4-[(2,2,5,5-tetramethyl-1-oxy-3-pyrrolidinyl)methoxy]-1,2,5-thiadiazole 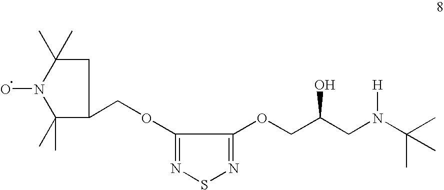
9. (S)-(−)-3-(3-tert-butylamino-2-hydroxypropoxy)-4-(2-hydroxymethyl-N-morpholino)-1,2,5-thiadiazole 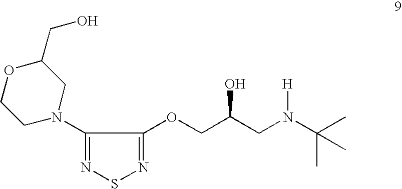
10. (S)-(−)-3-(3-tert-butylamino-2-hydroxypropoxy)-4-(2-nitratomethyl-N-morpholino)-1,2,5-thiadiazole 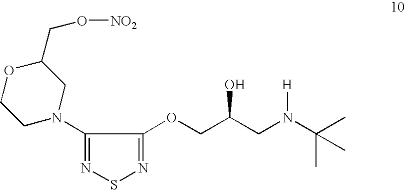
11. (S)-(−)-3-(3-tert-butylamino-2-hydroxypropoxy)-4-(2-hydroxymethyl-6-oxy-5,5,7,7-tetramethyl-hexahydropyrrolo[3,4-b][1,4]oxazin-4-yl)-1,2,5-thiadiazole 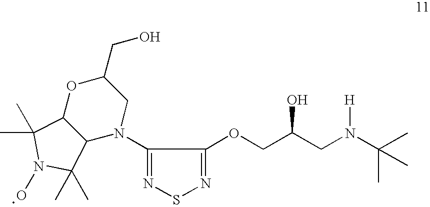
12. (S)-(−)-3-(3-tert-butylamino-2-hydroxypropoxy)-4-(2-nitratomethyl-6-oxy-5,5,7,7-tetramethyl-hexahydropyrrolo[3,4-b][1,4]oxazin-4-yl)-1,2,5-thiadiazole 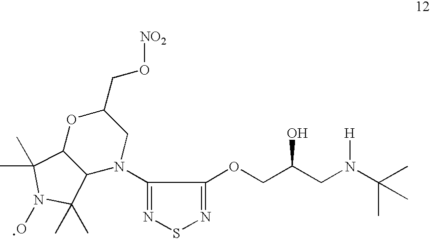
13. (S)-(−)-3-(3-tert-butylamino-2-nitratopropoxy)-4-(2-nitratomethyl-6-oxy-5,5,7,7-tetramethyl-hexahydropyrrolo[3,4-b][1,4]oxazin-4-yl)-1,2,5-thiadiazole 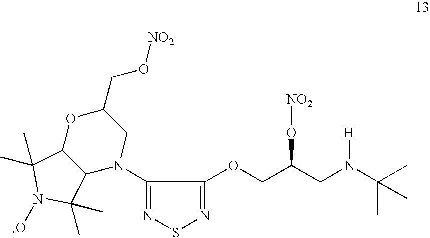
14. (S)-(−)-3-(3-tert-butylamino-2-hydroxypropoxy)-4-[(4-hydroxymethyl-2,2,5,5-tetramethyl-1-oxy-3-pyrrolidinyl)sulfanyl]-1,2,5-thiadiazole 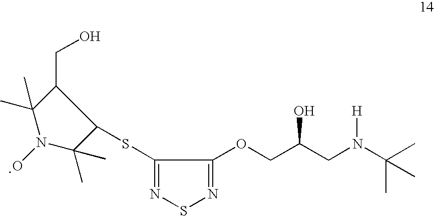
15. (S)-(−)-3-(3-tert-butylamino-2-hydroxypropoxy)-4-[(4-nitratomethyl-2,2,5,5-tetramethyl-1-oxy-3-pyrrolidinyl)sulfanyl]-1,2,5-thiadiazole 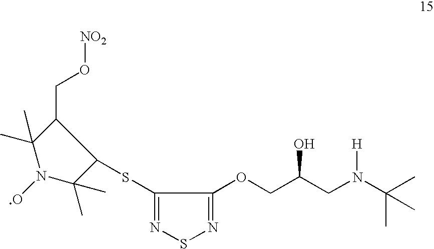
16. (S)-(−)-3-(3-tert-butylamino-2-nitratopropoxy)-4-[(4-nitratomethyl-2,2,5,5-tetramethyl-1-oxy-3-pyrrolidinyl)sulfanyl]-1,2,5-thiadiazole 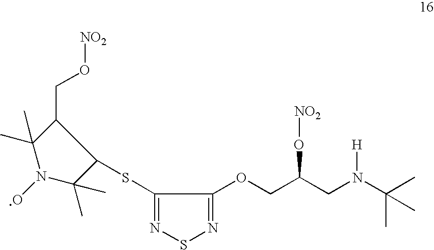
   Methods of Use for Multifunctional Beta-Antagonist Compounds

The present invention provides the multifunctional β-antagonist compounds for use in treatment of cardiovascular, ocular and other conditions for which treatment with beta-antagonists is indicated, as described herein.

The present invention further provides the use of the multifunctional β-antagonist compounds and functionalized beta-antagonist compounds of the present invention in the manufacture of a medicament for the treatment of cardiovascular conditions involving ischemia, angina, hypertension, palpitations, arrhythmias (e.g., supraventricular, ventricular), cardiomyopathy, congestive heart failure, as well as other conditions for which the use preceptor blocking agents have proven beneficial (e.g., symptoms associated with hyperthyroidism, anxiety (e.g., stage fright, examination apprehension, etc.), tremor (e.g., muscle tremor), migraine, alcohol withdrawal, tachycardia (e.g., as with thyrotoxicosis, pheochromocytoma, reflex tachycardia), esophageal varices, and ocular conditions (e.g., glaucoma or conditions associated with excess intraocular fluid).

The multifunctional beta-antagonist compounds may also be employed in the treatment of conditions associated with endothelial dysfunction or oxidative stress including cardiovascular diseases (such as ischaemic heart disease, angina pectoris, myocardial infarction, congestive heart failure, atherosclerosis, hypertension (e.g., pulmonary, systemic, ocular, obesity or pregnancy-induced), and management of arrhythmia (including but not limited to supraventricular arrhythmias, atrial tachycardia).

The relationship between reactive oxygen species (ROS) and nitric oxide (NO) plays a detrimental role in the modulation of many biological processes including aging, atherosclerosis, hypertension, diabetes mellitus, degenerative conditions, carcinogenesis, ischemia-reperfusion tissue injury, and acute and chronic inflammatory conditions. This is especially true in the case of cardiovascular conditions in general, and in hypertension and in particular, as well as in other conditions as indicated above (e.g., thyrotoxicosis and migraine). This is conceivable since oxidative stress exerted by ROS has been shown to significantly participate in the pathogenesis of hypertension and its related complications (i.e., IHD, CHF, RF, impotence, etc.,).

The production of NO is generally increased during atherosclerosis and hypertension. In addition to NO, these conditions are often referred to as oxidative stress-mediated diseases, where even higher increases in the production of superoxide and other ROS accompany the elevated production of NO. The eventual fate of NO is oxidation to nitrite (NO₂⁻) and nitrate (NO₃⁻), which are both end-products of NO metabolism under normal conditions. However, under oxidative stress conditions, besides the depletion of the natural antioxidant capacity, the major metabolic pathway of NO involves reaction with superoxide, resulting in the formation of a highly potent ROS, peroxynitrite. Peroxynitrite is an extremely hazardous ROS capable of interrupting many physiological functions.

Much progress has been made in our understanding of the role of the antioxidant enzymes, especially those involved in neutralizing superoxide (i.e., superoxide dismutase, SOD), in mediating the tissue resistance against oxidative stress and free radical injury.

In hypertension, for example, current therapies aim either to affect a certain system (e.g., rennin-angiotensin-aldosterone system, RAAS) or to target a specific receptor/s (beta-receptor, alpha-receptor, angiotensin receptor) to reduce elevated blood pressure. However, none of these therapeutic modalities has been shown to adequately affect the natural course of the disease or its outcome as evident by the still high incidence of morbidity and mortality associated with hypertension and its complications. This is conceivable since none of the current therapies address the multifactorial nature of the disease. In essence, however, many oxidative stress-mediated diseases like, for example, hypertension, can be described as a condition initiated by a yet unexplained hypersensitivity response of the vascular system to both endogenous and exogenous vasoconstritors. As explained above, this simplified sequence of events leading to essential hypertension is accompanied by a significant increase of ROS production (oxidative stress) that is accompanied by decreased biological activity of the major vasodilator NO. Logically, therefore, for a candidate drug to be effective, it has to adequately address as many events as possible of this sequence.

The present invention is especially applicable in the treatment of conditions including, but not limited to, the ocular conditions, cardiovascular conditions and other conditions disclosed herein. As used herein, and as well-understood in the art, “treatment” is an approach for obtaining beneficial or desired results, including clinical results. For purposes of this invention, beneficial or desired clinical results can include one or more, but are not limited to, alleviation or amelioration of one or more symptoms, diminishment of extent of disease, stabilized (i.e., not worsening) state of disease, preventing spread of disease, delay or slowing of disease progression, amelioration or palliation of the disease state, and remission (whether partial or total), whether detectable or undetectable.

In particular embodiments, multifunctional beta-antagonist compounds where the beta-antagonist component is betaxol, carteolol, levobunalol, metipranolol or timolol may be used for the treatment of ocular conditions, particularly those ocular conditions associated with an excess of intraocular fluid, for example, glaucoma.

In particular embodiments, multifunctional beta-antagonist compounds where the beta-antagonist component is propranolol or timolol may be used for the treatment of migraines.

Formulations and Dosage

The compounds can be provided in a variety of formulations and dosages. The compounds may be provided in a pharmaceutically acceptable form and/or in a salt form.

In one embodiment, the compounds are provided as non-toxic pharmaceutically acceptable salts. Suitable pharmaceutically acceptable salts of the compounds of this invention include acid addition salts such as those formed with hydrochloric acid, fumaric acid, p-toluenesulphonic acid, maleic acid, succinic acid, acetic acid, citric acid, tartaric acid, carbonic acid or phosphoric acid. Salts of amine groups may also comprise quaternary ammonium salts in which the amino nitrogen atom carries a suitable organic group such as an alkyl, alkenyl, alkynyl or aralkyl moiety. Furthermore, where the compounds of the invention carry an acidic moiety, suitable pharmaceutically acceptable salts thereof may include metal salts such as alkali metal salts, e.g., sodium or potassium salts; and alkaline earth metal salts, e.g., calcium or magnesium salts.

“Pharmaceutically-acceptable salt” refers to any salt of a compound of this invention which retains its biological properties and which is not biologically or otherwise undesirable. Such salts may be derived from a variety of organic and inorganic counter-ions well known in the art and include, by way of example illustration, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like. The term “pharmaceutically-acceptable cation” refers to a pharmaceutically acceptable cationic counter-ion of an acidic functional group. Such cations are exemplified by sodium, potassium, calcium, magnesium, ammonium, tetraalkylammonium cations, and the like.

The pharmaceutically acceptable salts of the present invention may be formed by conventional means, such as by reacting the free base form of the product with one or more equivalents of the appropriate acid in a solvent or medium in which the salt is insoluble, or in a solvent such as water which is removed in vacuo or by freeze drying or by exchanging the anions of an existing salt for another anion on a suitable ion exchange resin.

The present invention includes within its scope solvates of the multifunctional β-antagonist compounds and salts thereof, for example, hydrates.

The multifunctional β-antagonist compounds may have one or more asymmetric centers, and may accordingly exist both as enantiomers and as diastereoisomers. It is to be understood that all such isomers and mixtures thereof are encompassed within the scope of the present invention.

Additionally, all geometric isomers of the multifunctional beta-antagonists compounds of Formula I are included within the scope of this invention including, for example, all isomers with NO-donor and superoxide functionality.

The multifunctional β-antagonist compounds may be administered by oral, parenteral (e.g., intramuscular, intraperitoneal, intravenous, ICV, intracisternal injection or infusion, subcutaneous injection, or implant), buccal, vaginal, rectal, sublingual, or topical routes of administration and may be formulated, alone or together, in suitable dosage unit formulations containing conventional non-toxic pharmaceutically acceptable carriers, adjuvants, excipients and vehicles appropriate for each route of administration. In addition to the treatment of warm-blooded animals such as mice, rats, horses, cattle, sheep, dogs, cats, monkeys, etc., the compounds of the invention may be effective in humans.

The pharmaceutical compositions for the administration of the multifunctional β-antagonist compounds may conveniently be presented in dosage unit form and may be prepared by any of the methods well known in the art of pharmacy. The pharmaceutical compositions can be, for example, prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired therapeutic effect.

The pharmaceutical compositions containing the multifunctional β-antagonist compound as active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in the U.S. Pat. Nos. 4,256,108; 4,166,452; and 4,265,874 to form osmotic therapeutic tablets for control release. The pharmaceutical compositions of the invention may also be in the form of oil-in-water emulsions.

The present invention further provides use of the multifunctional α-antagonist compounds and functionalized beta-antagonist compounds of the present invention in the manufacture of a medicament for the treatment of cardiovascular conditions involving ischemia, angina, hypertension, palpitations, arrhythmias (e.g., supraventricular, ventricular), cardiomyopathy, congestive heart failure, as well as other conditions for which the use β-receptor blocking agents have proven beneficial (e.g., symptoms associated with hyperthyroidism, anxiety (e.g., stage fright, examination apprehension, etc.), tremor (e.g., muscle tremor), migraine, alcohol withdrawal, tachycardia (e.g., as with thyrotoxicosis, pheochromocytoma, reflex tachycardia), esophageal varices, and ocular conditions (e.g., glaucoma or conditions associated with excess intraocular fluid).

As described above, and as known in the art, pharmaceutical compositions for oral administration can take the form of bulk liquid solutions or suspensions, or bulk powders. More commonly, however, such compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampoules or syringes of the liquid compositions or pills, tablets, capsules or the like in the case of solid compositions. In such compositions, the nitrone compound is usually a minor component (from about 0.1 to about 50% by weight or preferably from about 1 to about 40% by weight) with the remainder being various vehicles or carriers and processing aids helpful for forming the desired dosing form.

Liquid forms suitable for oral administration may include a suitable aqueous or nonaqueous vehicle with buffers, suspending and dispensing agents, colorants, flavors and the like. Solid forms may include, for example, any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

In some embodiments, the composition comprising multifunctional beta-antagonist compounds where the beta-antagonist component is propranolol or timolol is formulated for oral administration.

The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent. Among the acceptable vehicles and solvents that may be employed are water, Ringer's solution and isotonic sodium chloride solution. The multifunctional β-antagonist compounds may also be administered in the form of suppositories for rectal administration of the drug.

For topical use (e.g., for the treatment of ocular conditions), solutions or suspensions, etc., containing the multifunctional β-antagonist compounds may be employed. Topical compositions are typically formulated as a topical suspension or solution (e.g., eye drops) containing the active ingredient(s), generally in an amount ranging from about 0.01 to about 20% by weight, preferably from about 0.1 to about 10% by weight, and more preferably from about 0.5 to about 15% by weight. Such topical formulations are well-known in the art. All such known topical formulations and ingredients are included within the scope of this invention.

In some embodiments, the composition comprising multifunctional beta-antagonist compounds where the beta-antagonist component is betaxol, carteolol, levobunalol, metipranolol or timolol is formulated for topical administration.

As those skilled in the art will recognize, the formulation of multifunctional β-antagonist compounds, the quantity of the formulation delivered, and the duration of administration of a single dose depend on the type of inhalation device employed as well as other factors. The frequency of administration and length of time for which the system is activated will depend mainly on the concentration of multifunctional β-antagonist compounds in the formulation. For example, shorter periods of administration can be used at higher concentrations of multifunctional β-antagonist compounds.

A liquid formulation can be manufactured by dissolving the multifunctional β-antagonist compounds in a suitable solvent, such as water, at an appropriate pH, including buffers or other excipients.

As known by those of skill in the art, the preferred dosage of multifunctional β-antagonist compounds will depend on the age, weight, general health and severity of the respiratory condition of the individual being treated. Dosage may also need to be tailored to the sex of the individual. Dosage may also be tailored to individuals suffering from more than one condition or those individuals who have additional conditions which affect their general health and tolerance of treatment. Dosage, and frequency of administration of the multifunctional β-antagonist compound will also depend on whether the compounds are formulated for treatment of acute episodes of the condition or for the prophylactic treatment of the condition (e.g., as for migraines or anxiety). A skilled practitioner will be able to determine the optimal dose for a particular individual. Various formulations of the compounds and compositions described herein may be administered according to the variables described above. In particular, formulations for prophylactic treatment of a variety of conditions may be administered, daily, twice daily, thrice daily or four times daily and/or upon the occurrence of symptoms associated with the underlying condition. It is contemplated that individuals who are using a prophylactic formulation may on occasion need to administer doses in response to acute episodes of symptoms. Administration includes any of the methods or routes as described herein.

The multifunctional β-antagonist compounds as described herein may be administered to an individual in need thereof over a period of time consistent with treatment of the condition from which the individual suffers. In the case of periodic conditions, the treatment may be discontinued when the individual is no longer affecting by the condition or deemed to be no longer in need of the treatment by a skilled practitioner. Examples of such time periods include days, weeks or months. Where the condition is a congenital or chronic condition such as certain cardiovascular conditions and others, it is envisioned that the treatment with the compounds described herein will be administered for a period of weeks, months, years or decades. The methods as described herein also include the administration of combinations of the multifunctional β-antagonist compounds as described herein, or combinations of the compounds described herein and other drugs used in the treatment of the cardiovascular conditions and other conditions described herein described herein or symptoms associated with these conditions.

As described in greater detail above, and as known by those of skill in the art, generally, the multifunctional beta-antagonist compounds described herein are administered in a pharmaceutically effective amount. The amount of the multifunctional beta-antagonist compound actually administered will typically be determined by a physician, in the light of the relevant circumstances, including the condition to be treated, the chosen route of administration, the actual compound administered, the age, weight, and response of the individual patient, the severity of the patient's symptoms, and the like.

The pharmaceutical compositions comprising the multifunctional beta-antagonist compounds described herein can be administered by any suitable routes including, by way of illustration, those described herein, such as, oral, topical via the eye, rectal, subcutaneous, intravenous, intramuscular, and the like. Depending on the intended route of delivery, the multifunctional beta-antagonist compounds are preferably formulated as either oral, topical or injectable compositions.

Injectable compositions are typically based upon injectable sterile saline or phosphate-buffered saline or other injectable carriers known in the art. As before, the multifunctional beta-antagonist compound in such compositions is typically a minor component, often being from about 0.05 to 10% by weight with the remainder being the injectable carrier and the like.

The above-described components for orally and topically administrable or injectable compositions are merely representative. Other materials as well as processing techniques and the like are set forth in Part 8 of Remington's Pharmaceutical Sciences, 18th edition, 1990, Mack Publishing Company, Easton, Pa., 18042, which is incorporated herein by reference.

The compounds of this invention can also be administered in sustained release forms or from sustained release drug delivery systems. A description of representative sustained release materials can be found in the incorporated materials in Remington's Pharmaceutical Sciences, supra.

Also provided are kits for administration of the multifunctional β-antagonist compound or composition comprising at least one multifunctional β-antagonist compound, that may include a dosage amount of at least one multifunctional β-antagonist compound or a composition comprising at least one multifunctional β-antagonist compound as disclosed herein. Kits may further comprise suitable packaging and/or instructions for use of the compound. Kits may also comprise a means for the delivery of the at least one multifunctional β-antagonist compound or compositions comprising at least one multifunctional β-antagonist compound, such as tube or other delivery means for eye drops, or pressure pack for capsules, tablets, or other device as described herein.

In another aspect of the invention, kits for treating an individual who suffers from or is susceptible to cardiovascular conditions and other conditions described herein are provided, comprising a container comprising a dosage amount of an multifunctional β-antagonist compound or composition as disclosed herein, and instructions for use.

Kits may also be provided that contain sufficient dosages of the multifunctional β-antagonist compound or composition to provide effective treatment for an individual for an extended period, such as a week, 2 weeks, 3, weeks, 4 weeks, 6 weeks or 8 weeks or more.

The invention is further illustrated by the following nonlimiting examples.

All patents, patent applications and publications referred to herein are hereby incorporated herein by reference in their entirety.

### EXAMPLES

In the examples below, the following abbreviations have the following meanings. Abbreviations not defined below have their generally accepted meaning.

bd=broad doublet

bs=broad singlet

d=doublet

dd=doublet of doublets

dec=decomposed

dH₂O=distilled water

ELISA=enzyme-linked immuno-sorbent assay

eq=equivalent

EtOAc=ethyl acetate

EtOH=ethanol

g=grams

h=hours

Hz=hertz

ip=intraperitoneal

L=liter

m=multiplet

min=minutes

M=molar

MeOH=methanol

mg=milligram

MHz=megahertz

mL=milliliter

mmol=millimole

m.p.=melting point

N=normal

po=per os, oral

q=quartet

quint.=quintet

s=singlet

t=triplet

THF=tetrahydrofuran

TLC=thin layer chromatography

μg=microgram

μL=microliter

UV=ultraviolet

In the examples below, the temperatures are in degrees Celsius.

### Example 1

### Preparation of 2-tert-butylamino-1-(5,5,7,7-tetramethyl-6-oxy-6,7-dihydro-5H-1-oxa-6-aza-s-indacen-2-yl)ethyl nitrate

A solution of equivalent amount of dimethyl 4-methoxyphthalate and benzylamine in toluene was refluxed under argon atmosphere with a Dean-Stark trap to remove the methanol generated. The reaction was continued until the boiling point of the refluxing solvents remained at 111° C. indicating no further generation of methanol. Removal of solvent by rotary evaporation gave a residue that was purified by column chromatography and recrystallization to provide 2-benzyl-5-methoxyphthalimide.

To a solution of 2-benzyl-5-methoxyphthalimide in dry toluene was added dropwise an ether solution of methylmagnesium iodide (6 eq) under argon atmosphere. Ether was removed by slow distillation and the remaining mixture was refluxed at 111° C. for 4-6 h under argon gas. The mixture was cooled to 0° C. with an ice bath and a water solution saturated with NH₄Cl was carefully added with stirring. The resulting mixture was stirred for a while and ethyl acetate was added. The mixture was filtered through Celite and the organic layer was separated and evaporated. The residue was purified by column chromatography and recrystallization to give 2-benzyl-5-methoxy-1,1,3,3-tetramethylisoindoline.

Under argon protection, 10% Pd/C was added in portions to ethanol and then a solution of 2-benzyl-5-methoxy-1,1,3,3-tetramethylisoindoline was added. The mixture was shake under 50 psi of hydrogen at room temperature for 15 h and then filtered. Removal of solvent gave 5-methoxy-1,1,3,3-tetramethylisoindoline that was pure enough for further reaction.

To a solution of 5-methoxy-1,1,3,3-tetramethylisoindoline in water and ethanol were added sodium tungstate dihydrate and hydrogen peroxide. The solution was stirred at room temperature until the completion of the reaction as monitored by TLC. Sodium chloride-saturated water solution was added and the mixture was extracted with ethyl acetate. The crude residue was purified by column chromatography and recrystallization to afford 5-methoxy-2-oxy-1,1,3,3-tetramethylisoindoline as an orange solid.

To a solution of 5-methoxy-2-oxy-1,1,3,3-tetamethylisoindoline in dichloromethane was added boron bromide. The reaction mixture was stirred at room temperature until the completion of the reaction. Water was added and the mixture was stirred for 45 min. Routine work-up gave a residue that was purified by column chromatography to generate 5-hydroxy-2-oxy-1,1,3,3-tetramethylisoindoline.

A solution of mole equivalent amounts of 5-hydroxy-2-oxy-1,1,3,3-tetramethylisoindoline and sodium ethoxide in DMF was stirred for 10 min and then bromoacetaldehyde diethyl acetal (1 eq) was added. The mixture was stirred at room temperature and monitored by TLC. After the completion of the alkylation reaction, 5% hydrochloric acid was added and the reaction mixture was stirred until the disappearance of the alkylated diethyl acetal. Normal work-up provided a residue that was purified by flash column chromatography to give 5-(formylmethoxy)-2-oxy-1,1,3,3-tetramethylisoindoline.

Sulfuric acid was used as the dehydrating agent to mix with 5-(formylmethoxy)-2-oxy-1,1,3,3-tetramethylisoindoline at room temperature. The mixture was stirred until the completion of the cyclization. The mixture was slowly poured into icy water and the pH was adjusted to 7 with sodium carbonate-saturated water. The mixture was extracted with dichloromethane. Removal of solvent provided a residue that was purified by flash column chromatography to produce the nitroxide benzofuran 5,5,7,7-tetramethyl-6-oxy-6,7-dihydro-5H-1-oxa-6-aza-s-indacene.

To a solution of 5,5,7,7-tetramethyl-6-oxy-6,7-dihydro-5H-1-oxa-6-aza-s-indacene in acetic anhydride at 0° C. was added boron fluoride and tin (IV) chloride. The reaction mixture was stirred at room temperature until the completion of the acetylation reaction as monitored by LC/MS or TLC. Then the mixture was slowly poured into icy water and the pH was adjusted to 7 with sodium bicarbonate. The aqueous solution was extracted with dichloromethane. Removal of the solvent gave a residue that was purified by flash column chromatography and recrystallization to provide the nitroxide acetyl furan 1-(5,5,7,7-tetramethyl-6-oxy-6,7-dihydro-5H-1-oxa-6-aza-s-indacen-2-yl)ethanone.

To a solution of 1-(5,5,7,7-tetramethyl-6-oxy-6,7-dihydro-5H-1-oxa-6-aza-s-indacen-2-yl)ethanone in acetic acid was added dropwise a solution of bromine (1 eq) in acetic acid at 0° C. The reaction was monitored by TLC or LC/MS. After the completion of the bromination reaction, the solvent was removed by rotary evaporation under vacuum. The residue was purified by flash column chromatography to give the bromoacetyl-compound 2-bromo-1-(5,5,7,7-tetramethyl-6-oxy-6,7-dihydro-5H-1-oxa-6-aza-s-indacen-2-yl)ethanone.

The bromoacetyl-compound 2-bromo-1-(5,5,7,7-tetramethyl-6-oxy-6,7-dihydro-5H-1-oxa-6-aza-s-indacen-2-yl)ethanone was reduced with borane compounds such as B-chlorodiisopinocampheylborane or borane/oxazaborolidine (R or S) in THF at −30 to −5° C. to produce nonchiral or chiral intermediate 2-bromo-1-(5,5,7,7-tetramethyl-6-oxy-6,7-dihydro-5H-1-oxa-6-aza-s-indacen-2-yl)ethanol, which was purified by flash column chromatography.

A solution of 2-bromo-1-(5,5,7,7-tetramethyl-6-oxy-6,7-dihydro-5H-1-oxa-6-aza-s-indacen-2-yl)ethanol in THF was added to another solution of tert-butylamine in THF at room temperature. The solution was refluxed under argon atmosphere until the disappearance of the bromo compound as monitored by TLC or LC/MS. Then sodium borohydride was added and the mixture was stirred for several hours. Removal of the solvent gave a residue that was dissolved in water and extracted with dichloromethane. The solvent was removed and the residue was purified by flash column chromatography to yield the product 2-tert-butylamino-1-(5,5,7,7-tetramethyl-6-oxy-6,7-dihydro-5H-1-oxa-6-aza-s-indacen-2-yl)ethanol.

To a solution of 2-tert-butylamino-1-(5,5,7,7-tetramethyl-6-oxy-6,7-dihydro-5H-1-oxa-6-aza-s-indacen-2-yl)ethanol in sulfuric acid at 0° C. was added dropwise nitric acid. The solution was stirred at 0° C. for 1 h and then poured into icy water. The pH was adjusted to 8 and the mixture was extracted with chloroform. Removal of solvent gave a residue that was purified by flash column chromatography to generate the final title compound.

### Example 2

### Preparation of 2-isopropylamino-1-(5,5,7,7-tetramethyl-6-oxy-6,7-dihydro-5H-1-oxa-6-aza-s-indacen-2-yl)ethyl nitrate

The title compound was made by the same methods used for the preparation of compound (14) as described in Example 1 except using isopropylamine instead of tert-butylamine.

### Example 3

### Preparation of 2-tert-butylamino-1-(5,5,7,7-tetramethyl-6-oxy-6,7-dihydro-5H-1-oxa-6-aza-s-indacen-2-yl)-1-nitrosothioethane HCl salt

The nitroxide benzofuran bromoethanol 2-bromo-1-(5,5,7,7-tetramethyl-6-oxy-6,7-dihydro-5H-1-oxa-6-aza-s-indacen-2-yl)ethanol was treated with sodium hydride in tetrahydrofuran to provide the corresponding epoxide 5,5,7,7-tetramethyl-2-oxiranyl-6-oxy-6,7-dihydro-5H-1-oxa-6-aza-s-indacene that was purified by flash column chromatography.

The epoxide 5,5,7,7-tetramethyl-2-oxiranyl-6-oxy-6,7-dihydro-5H-1-oxa-6-aza-s-indacene was added to a solution of sodium thiocyanate in aqueous ethanol or a solution of potassium thiocyanate in aqueous dioxane. The solution was vigorously stirred at room temperature for 36 h. Sodium chloride-saturated water was added and the resulting mixture was extracted with dichloromethane. Removal of solvent yielded a residue that was purified by flash column chromatography to provide the thiirane 5,5,7,7-tetramethyl-2-thiiranyl-6-oxy-6,7-dihydro-5H-1-oxa-6-aza-s-indacene.

A mixture of tert-butylamine (10 mmol) and N,O-bis(trimethylsilyl)acetamide (1 mL) in DMSO (50 mL) was stirred under argon at room temperature for 30 min. A solution of the thiirane 5,5,7,7-tetramethyl-2-thiiranyl-6-oxy-6,7-dihydro-5H-1-oxa-6-aza-s-indacene in DMSO (50 mL) was added and the solution was heated at 80° C. for 48 h. The mixture was cooled and the solvent was vacuum distilled (0.1 mm). The thiol-containing residue was purified by flash column chromatography to generate the material 2-tert-butylamino-1-(5,5,7,7-tetramethyl-6-oxy-6,7-dihydro-5H-1-oxa-6-aza-s-indacen-2-yl)ethanethiol that was dissolved in 8N HCl (10 mL), stirred and cooled in an ice bath. A solution of sodium nitrite (3 eq) in water was added to the acidic solution of the thiol compound and the mixture was stirred at 0-5° C. until the completion of the reaction. The precipitated solid of the title compound 2-tert-butylamino-1-(5,5,7,7-tetramethyl-6-oxy-6,7-dihydro-5H-1-oxa-6-aza-s-indacen-2-yl)-1-nitrosothioethane HCl salt was collected by filtration, washed twice with cold diluted hydrochloric acid, dried in a dessicator and stored in airtight containers under nitrogen at −20° C. until use.

### Example 4

### Preparation of 2-isopropylamino-1-(5,5,7,7-tetramethyl-6-oxy-6,7-dihydro-5H-1-oxa-6-aza-s-indacen-2-yl)-1-nitrosothioethane HCl salt

The title compound was made by the same methods used for the preparation of compound 20 as described in Example 3 except using isopropylamine instead of tert-butylamine.

### Example 5

### Preparation of (S)-(−)-3-(3-tert-butylamino-2-hydroxypropoxy)-4-chloro-1,2,5-thiadiazole (17)

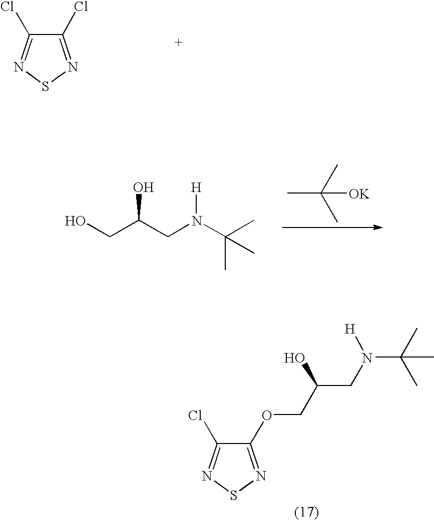

A mixture of (S)-(−)-3-(3-tert-butylamino)-1,2-propanediol, (2.0 g, 13.59 mole) and 3,4-dichloro-1,2,5-thiadiazole (2.11 g, 13.59 mole) in anhydrous tert-butanol (10 mL) was heated to reflux under nitrogen. Potassium tert-butoxide solution in tert-butanol (1.0 M, 13.6 mL, 13.6 mmole) was then added and the mixture was refluxed for 60 min. The mixture was cooled to room temperature and filtered. The filtrate was concentrated to give brown-red oil (3.49 g, yield 96.6%). Purity by LC was 93.2% at 254 nm. MS (ESL %): m/z=266 (M+1, 100)

### Example 6

### Preparation of (S)-(−)-3-(3-tert-butylamino-2-hydroxypropoxy)-4-[(2,2,5,5-tetramethyl-1-oxy-3-pyrrolidinyl)methoxy]-1,2,5-thiadiazole (8)

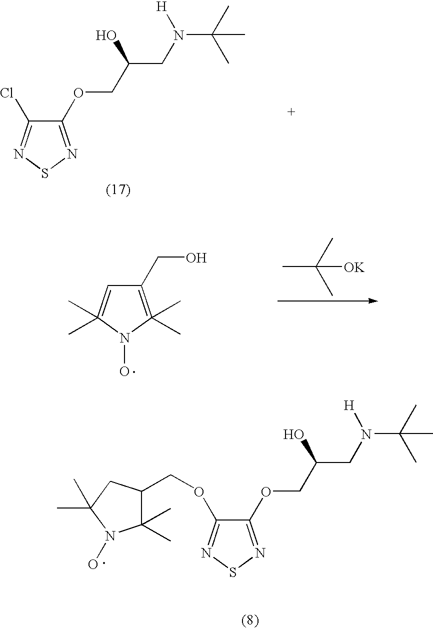

A mixture of equivalent amount of 3-hydroxymethyl-2,2,5,5-tetramethyl-pyrrolidinyl-1-oxy and (S)-(−)-3-(3-tert-butylamino-2-hydroxypropoxy)₄-chloro-1,2,5-thiadiazole in anhydrous tert-butanol was heated to reflux under nitrogen. Potassium tert-butoxide (one equivalent) was then added and mixture refluxed for 60 min. The mixture was cooled to room temperature and filtered. The filtrate was concentrated and purified by column chromatography.

### Example 7

### Preparation of (S)-(−)-3-(3-tert-butylamino-2-nitratopropoxy)-4-[(2,2,5,5-tetramethyl-1-oxy-3-pyrrolidinyl)methoxy]-1,2,5-thiadiazole (7)

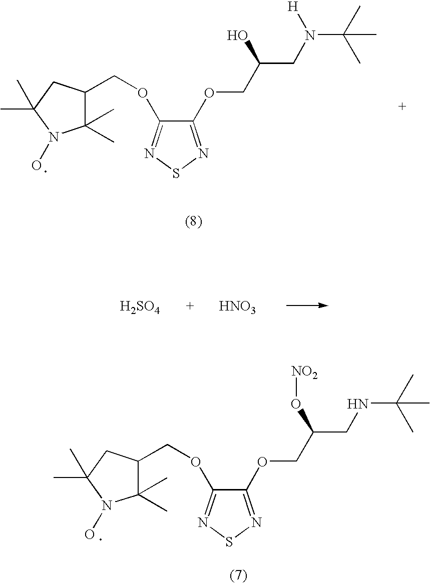

Compound II (2.0 g) is added portion-wise to 10 mL of concentrated sulfuric acid. While cooling in ice bath concentrated nitric acid (10 mL) is added drop-wise. After addition of nitric acid is complete, continue stirring at room temperature for an additional 15 min and cool again on ice bath. The reaction content is slowly poured into a pre-prepared mixture of ether, petroleum ether, triethylamine and crushed ice. After addition is complete, the pH is adjusted to 8-9. The mixture is extracted with chloroform, dried and concentrated to give the residue which is purified by column chromatography.

### Example 8

### Preparation of (S)-(−)-3-(3-tert-butylamino-2-hydroxypropoxy)-4-(2-hydroxymethyl-N-morpholino)-1,2,5-thiadiazole (9)+

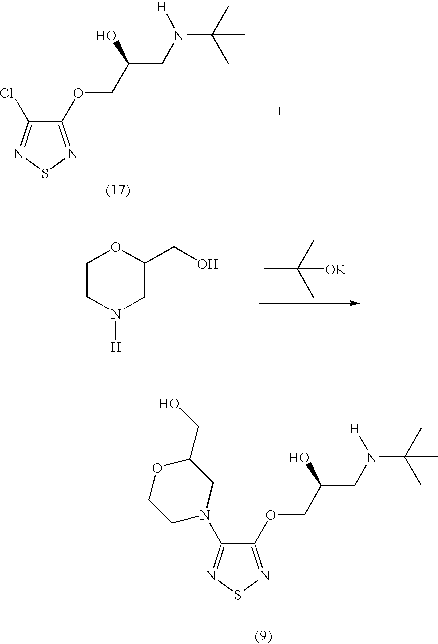

A mixture of equivalent amount of (S)-(−)-3-(3-tert-butylamino-2-hydroxypropoxy)₄-chloro-1,2,5-thiadiazole and 2-hydroxymethylmorpholine in anhydrous tert-butanol was heated to reflux under nitrogen. Potassium tert-butoxide (one equivalent) was then added and mixture refluxed for 60 min. The mixture was cooled to room temperature and filtered. The filtrate was concentrated and purified by column chromatography.

### Example 9

### Measurement of NO-Donating Properties

(a) NO-Induced Formation of cGMP

Nitric oxide (NO) stimulates guanylate cyclase in smooth muscle cells, which results in the formation of cyclic GMP. Primary cultures of rat aortic smooth muscle cells (RAOSMC) is used to measure cyclic GMP generated in the presence of NO-donors. Primary cultures (passage 2) of RAOSMC are purchased from Cell Applications and grown in 6-well dishes (9.5 cm²; Costar) in growth media of DMEM/F12 (1:1) supplemented with 10% fetal bovine serum (cyclone), 2 mM 1-glutamine, 100 U/ml penicillin, and 100 μg/ml streptomycin in a 37° C. incubator in an atmosphere of 5% CO₂-95% air. Cells are assayed at 90% confluency. At the time of assay, growth media is replaced with warm (37° C.) assay buffer (Hanks BSS containing 10 mM Hepes and 0.1% BSA, pH 7.5). The phophodiesterase inhibitor zaprinast (30 μM; Calbiochem) is added for 15 minutes prior to addition of test drug. The test drug is added and the cells incubated at 37° C. for 15 min. The assay is stopped by aspiration of assay buffer, and addition of 0.4 ml of cold 0.1 M HCl. The dishes are incubated for 15 min at 4° C., and the cell lysate scraped and transferred to a microfuge tube on ice. The tube is centrifuged for 4 min at 12,000 RPM at 4° C., and the supernatant assayed for cyclic GMP content by ELISA using the acetylation protocol as described by the kit manufacturer (Assay Designs, Ann Arbor, Mich.).

(b) Measurement of Nitrate and Nitrite Formed from NO

Nitric oxide (NO) is rapidly converted to nitrate and nitrite in aqueous solution. Subsequent enzymatic conversion of nitrate to nitrite, followed by calorimetric determination of nitrite concentrations, is used to determine the amount of NO produced in solution. This assay will measure the production of NO by test compounds in the presence of cells which can metabolize organic nitrates to NO. Cells used is primary cultures of RAOSMC (see above). Cells (passage 3-6) are grown in 24-well dishes (1.9 cm²; Costar) in growth media of 0.5 ml DMEM/F12 (1:1) supplemented with 10% fetal bovine serum (Hyclone), 2 mM 1-glutamine, 100 U/ml penicillin, and 100 μg/ml streptomycin in an atmosphere of 5% CO₂-95% air at 37° C. Cells are assayed at 90% confluency. Test compounds are added to the media, and the cells incubated for 2-24 hours in an atmosphere of 5% CO₂-95% air at 37° C. At the end of this incubation, the assay media is collected and assayed for NO production. Nitrate is converted to nitrite by adding 10 μl of nitrate reductase (1 U/ml) and 10 μl of NADH (2 mM) to 80 μl of assay media or standard, which is then incubated for 30 min at 37° C. Nitrite is quantitated by adding 100 μl of Greiss reagent (1:1 mixture of Greiss reagent I and Greiss reagent II prepared just before assay) and measuring optical density at 550 nm. Standards of sodium nitrate and sodium nitrite (1-100 μM) are made in growth media and processed as described for cell media samples.

(c) Measurement of NO-Induced Relaxation of Rat Aorta

Nitric oxide (NO) induces the rapid relaxation of precontracted vascular smooth muscle, and this assay is used to measure NO generated from test compounds in the presence of blood vessels. Male Sprague-Dawley rats (200-250 g) are purchased from Comparative Biosciences (Mountain View, Calif.) and used in the rat aortic rings relaxation studies in a tissue bath preparation. Thoracic aorta are removed following anesthesia with i.p. injection of ketamine (50 mg/kg) and xylazine (10 mg/ml). The adventia surrounding the vessel is carefully removed, and the aorta is cut into rings of 4-5 mm and mounted in the tissue bath (5 ml volume). Kreb's-Henseleit buffer is used as the tissue bath buffer, and it is constantly gassed with carbogen and maintained at 37° C. The rings are preloaded with 2 g tension and equilibrated for 90 min with buffer changed every 15 min. After stabilization, the rings are contracted with phenylephrine (PE; 0.3 μM). Dose-response curves for the relaxation of PE-contracted rings are performed by cumulative addition of test drug. After the last addition of test drug, sodium nitroprusside (1 μM) is added to induce complete relaxation of the ring. Values are expressed as the percent of maximal tension induced by 0.3 μM PE.

### Example 10

### Affinity of Multifunctional Beta-Antagonist Compounds for Beta-Adrenergic Receptors

The affinity of test compounds for beta-adrenergic receptors is determined using a radio ligand binding assay. The tissue used is C6 glioma cell membranes, which contain both beta-1- and beta-2-adrenergic receptors. The radioligand used is [¹²⁵I]-iodopindolol (NEN). C6 glioma cells are grown in 100 mm dishes in DMEM supplemented with 5% FBS (Hyclone) in an atmosphere of 10% CO₂-90% air at 37° C. At confluency, cells are lysed with cold hypotonic buffer (1 mM HEPES, 2 mM EDTA, pH 7.4), scraped off, and centrifuged at 20,000×g for 20 min. The pellet is resuspended in 20 mM Tris-HCl and 0.9% NaCl, and stored at −70° C. For binding assays, aliquots of the thawed membrane preparation were added to assay tubes containing, in a final volume of 0.25 ml, 12 mM Tris-HCl (pH 7.4), 0.54% NaCl, 1.1 mM ascorbic acid, 0.0004% BSA, [¹²⁵I]-IPIN (0.15 nM final), and test compound. Nonspecific binding is defined using 0.3 μM (−) propranolol. Incubations are carried out for 25 min at 37° C. and are stopped by addition of 10 ml of 0.9% NaCl, 10 mM Tris-HCl (pH 7.4, 25° C.) to each assay tube. The samples are filtered through glass-fiber filters (Schleicher and Schuell No. 30) and washed with an additional 10 ml buffer. The radioactivity retained on the filter is counted in a gamma counter. Protein is determined by the Bradford method using BSA as a standard. Dose response curves for the inhibition of the binding, [¹²⁵I]-IPIN is analyzed by nonlinear regression analysis using the Prism software program (Prism 3.0; Graph Pad). Kd values for the binding of a competing drug is calculated from the IC50 values using the Cheng and Prusoff correction.

### Example 11

### Measurement of SOD-Mimetic Properties

Lucigenin is an acridylium dinitrate compound that emits light on reduction and interaction with the superoxide anion (O₂⁻), and is used to measure O₂⁻ production. Compounds are tested for their ability to scavenge O₂— generated by the reaction of xanthine+xanthine oxidase. Reduction of the lucigenin chemiluminescence signal in the presence of xanthine+xanthine oxidase is used as the measurement of O₂⁻ scavenging potency. The assay reaction buffer is Hank's BSS containing 20 μM Hepes (pH 7.4), 0.1% BSA, 250 μM lucigenin, 200 μM xanthine, and test compound. A vial containing 1.6 ml of the reaction mixture and 0.2 ml of test compound is placed in a liquid scintillation counter and dark adapted for 5 min. The reaction is started by addition of 0.2 ml of xanthine oxidase (0.0005 U/ml final), and emitted light is recorded continuously for 10 min. Superoxide dismutase (SOD; 0.5 U/ml final) is used as a positive control to completely inhibit the SO-specific signal. The light signal (cpm) at 5 minutes is used to compute the percent reduction of control response.

### Example 12

### Measurement of Anti-Hypertensive Properties In Vivo

The anti-hypertensive effect of the multi-functional compounds are assessed according to a method modified from that described by H. Gerhard Vogel Ed 1997 (In: Drug Discovery and Evaluation—Pharmacological assays; Chapter A.1.3; Springer Verlag). Briefly, Male Sprague-Dawley rats (200-250 g; purchased from Comparative Biosciences, Mountain View, Calif.) are anaesthetized with thiopentone sodium (120 mg/kg, i.p), trachea is cannulated to facilitate spontaneous respiration and the rectal temperature is maintained at 37° C. with a homeothermic blanket system (Harvard Apparatus, Holliston, Mass.). The right carotid artery is cannulated and connected to a pressure transducer (SensNor 840, Horten, Norway) for the measurement of arterial blood pressure (systolic, diastolic, mean arterial) and heart rate which are recorded for the duration of the experiment and displayed on a PowerLab 8 recording system (AD Instruments, Colorado, USA). The left jugular vein is cannulated for the administration of drugs. The response to drugs are quantified as either absolute change in blood pressure (mmHg) (or heart rate, bpm (beats per minute)) or area under the response curve (mean arterial blood pressure, mmHg.min) using the chart analysis software. When stable homodynamic conditions are achieved for at least 30 min, increasing bolus i.v. doses of isoprenaline (isoproterenol, 0.0 μg/kg, 0.10 μg/kg, 1 μg/kg) are administered after injection of test compounds (1, 10, or 30 mg/kg, i.v.) or the appropriate vehicle. The activity of the test compound was compared against the vehicle in affecting the blood pressure responses to ispoprenaline.

### Example 13

### Measurement of Changes in Intra-Ocular Pressure (IOP) in a Rabbit Model of Glaucoma

The ability of the multi-functional compounds to affect IOP in a model of glaucoma is assessed according to a method modified from that described by Sears and Sears (Am. J. Opthalm (1974) 77:378-383). Briefly, male New Zealand rabbits weighing about 2 kg are held in restrainers and are pre-treated with 10 mg/kg i.p. indomethacin to prevent the otherwise immediate onset of inflammation and then slightly anesthetized with pentobarbital (10 mg/kg, i.p.) to eliminate any nystagmus. The right eye is anaesthetized topically with 2% lidocaine. The anterior chamber is cannulated with a 30-gauge needle attached to a reservoir set at a pressure of 25 mmHg. Then a second cannula, 32-gauge, is introduced into the anterior chamber near the limbus and directed to the posterior chamber through the pupil. A sterile isotonic saline solution (0.5 ml) containing 150 units of alpha-chymotrypsin is irrigated through the cannula into the posterior chamber. Care is taken to avoid the injection of any enzyme into the corneal stroma. Both cannulae are then removed without significant loss of aqueous humor. The eyes are examined at daily intervals for the first week, and then on alternative days for the second week, and then weekly for the duration of the experiment. Intraocular pressure is measured with a tonometer connected to a computerized data recording equipment (Power Lab 8, AD Instruments, Colorado, USA) adapted for rabbit eyes. Effect of the test compounds (0.1%, 1%, 3% solution) on IOP is compared against the appropriate vehicle control following treatment before and after surgery.

### Example 14

### Pharmaceutical Formulations of Multifunctional Beta-Antagonist Compounds

The following formulations illustrate representative pharmaceutical compositions comprising multifunctional beta-antagonist compounds. These formulations are, however, illustrative and are not intended to limit the invention as claimed.

Formulation 1—Tablets

A multifunctional beta-antagonist compound is admixed as a dry powder with a dry gelatin binder in an approximate 1:2 weight ratio. A minor amount of magnesium stearate is added as a lubricant. The mixture is formed into 240-270 mg tablets (80-90 mg of active nitrone compound per tablet) in a tablet press.

Formulation 2—Capsules

A multifunctional beta-antagonist compound is admixed as a dry powder with a starch diluent in an approximate 1:1 weight ratio. The mixture is filled into 250 mg capsules (125 mg of active nitrone compound per capsule).

Formulation 3—Liquid

A multifunctional beta-antagonist compound (125 mg), sucrose (1.75 g) and xanthan gum (4 mg) are blended, passed through a No. 10 mesh U.S. sieve, and then mixed with a previously made solution of microcrystalline cellulose and sodium carboxymethyl cellulose (11:89, 50 mg) in water. Sodium benzoate (10 mg), flavor, and color are diluted with water and added with stirring. Sufficient water is then added to produce a total volume of 5 mL.

Formulation 4—Injection

The multifunctional beta-antagonist compound is dissolved in a buffered sterile saline injectable aqueous medium to a concentration of approximately 5 mg/mL.

Formulation 5—Ointment

Stearyl alcohol (250 g) and white petrolatum (250 g) are melted at about 75° C. and then a mixture of a multifunctional beta-antagonist compound (50 g), methylparaben (0.25 g), propylparaben (0.15 g), sodium lauryl sulfate (10 g), and propylene glycol (120 g) dissolved in water (about 370 g) is added and the resulting mixture is stirred until it congeals.

Disclosed and described, it is to be understood that this invention is not limited to the particular examples, process steps, and materials disclosed herein as such process steps and materials may vary somewhat. It is also to be understood that the terminology used herein is used for the purpose of describing particular embodiments only and not intended to be limiting since the scope of the present invention will be limited only by the appended claims and equivalents thereof. The preceeding examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

1-50. (canceled)

51. A method of treating or preventing a disorder selected from the group consisting of disorders in which treatment with a β-antagonist is indicated, disorders associated with oxidative stress and free radical injury, and disorders in which treatment with a smooth muscle relaxant is indicated, in a mammal in need thereof, comprising administering to said mammal an effective amount of a multifunctional β-blocker compound comprising
i) a β-blocker component,
ii) at least one reactive oxygen species (ROS) scavenger component, and optionally
iii) at least one nitric oxide (NO) donor component.

52. A method according to claim 51, wherein said multifunctional β-blocker compound comprises
i) a β-blocker component,
ii) at least one reactive oxygen species (ROS) scavenger component, and
iii) at least one nitric oxide (NO) donor component.

53. A method according to claim 51, wherein said β-blocker component is selected from the group consisting of compounds used in medicine as β-adrenergic blockers, derivatives thereof, and compounds exhibiting affinity for β-receptors.

54. A method according to claim 51, wherein said ROS-scavenger component comprises an antioxidant reacting with ROS selected from the group consisting of superoxide, hydroxyl radicals, peroxynitrite, and hypochlorite.

55. A method according to claim 51, wherein said NO-donor comprises a group capable of providing nitric oxide in a form selected from uncharged and charged.

56. A method according to claim 51, wherein said ROS-scavenger component comprises a substituted N-oxide free radical, or a substituted or unsubstituted lipoic acid moiety.

57. A method according to claim 51, wherein said ROS-scavenger component comprises any of alkenyl group, aryl group, substituted aryl group, sulfhydryl, dithiol in oxidized or reduced form, and group that is converted in vivo into a sulfhydryl in its oxidized or reduced form.

58. A method according to claim 56, wherein the N-atom of said N-oxide is a member of 3 to 7 membered heterocyclic ring.

59. A method according to claim 51, wherein said NO donor component comprises a group selected from —NO₂, —ONO, —SNO, and —NONOate.

60. A method according to claim 51, wherein said disorder is selected from the group consisting of cardiovascular, pulmonary, neurological, hormonal, and ocular.

61. A method according to claim 51, wherein said disorder is selected from the group consisting of ischemia, ischemia-reperfusion tissue injury, acute and chronic inflammatory conditions, angina, atherosclerosis, impotence, hypertension, pulmonary hypertension, systemic hypertension, obesity or pregnancy-induced hypertension, palpitations, arrhythmias, cardiomyopathy, congestive heart failure, hyperthyroidism, anxiety, tremor, migraine, alcohol withdrawal, tachycardia, thyrotoxicosis, pheochromocytoma, esophageal varices, glaucoma, conditions associated with excess intraocular fluid, diabetes mellitus, and carcinogenesis.

62. A method according to claim 51, further comprising treating or preventing an adverse effect caused by β-antagonists.

63. A method according to claim 62, wherein said adverse effect is selected from the group consisting of induced congestive hear failure, induced or exacerbated heart failure, acute myocardial infarction or cardiomegaly, blockage of β₂-receptors in bronchial smooth muscle, increasing airway resistance, fatigue, sleep disturbances, memory loss and depression, and complications associated with diabetes.

64. A method according to claim 51, wherein said β-blocker component is derived from a β-antagonist used in medicine selected from the group consisting of Acebutolol, Alprenolol (Aptin), Amosulalol, Arotinolol, Atenolol (Atehexal), Befunolol, Betaxolol, Bevantolol, Bisprolol (Zebeta), Bopindolol, Bucumolol, Bufetolol, Bufuralol, Bunitrolol, Bupranolol, Butidrine Hydrochloride, Butofilolol, Carazolol, Carteolol, Carvedilol (Coreg, Dilatrend, Kredex), Carvedilol, Celiprolol, Cetamolol, Cloranolol, Dilevalol, Disopryamide (Norpace), Epanolol, Esmolol, Indenolol, Labetalol, Levobunolol, Mepindolol, Metipranolol, Metohexal (Meijoprolol), Metoprolol (Betaloc), Metoprolol, Moprolol, Nadolol, Nadoxolol, Nebivolol, Nifenalol, Nipradilol, Oxprenolol (Corbeton), Penbutolol, Pindolol, Practolol, Pronethalol, Propranolol, Quinidine Gluconate (Quinaglute), Quinidine Polygalacturonate (Cardioquin), Quinidine Sulfate (Quinidex, Cin-quin), Sotalol (Sotocor, Sotahexal), Sulfinalol, Talinolol, Tertatolol, Tilisolol, Timolol, Toliprolol, Toprol XL, and Xibenolol.

65. A method according to claim 51, wherein said β-blocker component is derived from a β-antagonist selected from the group consisting of Carteolol, Oxprenolol, Nadolol, Propranolol, Metoprolol, Metipranol, Pindolol, Betaxolol, Atenolol, Esmololol, Levobunolol, Labetalol, and Tomolol.

66. A method according to claim 51, wherein said compound has Formula I 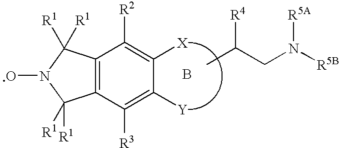
wherein R¹ may be independently hydrogen, substituted or unsubstituted alkyl, substituted or unsubstituted alkenyl, substituted or unsubstituted alkynyl, substituted or unsubstituted cycloalkyl, substituted or unsubstituted cycloalkenyl, substituted or unsubstituted alkoxy, substituted or unsubstituted aryloxy, substituted or unsubstituted aryl, or substituted or unsubstituted heterocycles, wherein the optional substituent may be a group capable of donating NO;
R² and R³ may be independently hydrogen or (CH₂)ₙX¹, n being from 0 to 4, and X¹ being H, OH, ═O (where n is not 0) or a group capable of donating NO, or
R² and R³ may be independently selected from H, OH, substituted or unsubstituted alkyl, substituted or unsubstituted alkenyl, substituted or unsubstituted alkynyl, substituted or unsubstituted cycloalkyl, substituted or unsubstituted cycloalkenyl, substituted or unsubstituted aryl, and substituted or unsubstituted heterocycle, wherein the optional substituent may be a group capable of donating NO;
R⁴ may be (CH₂)ₘX², m being from 0 to 4, and X² being H, SH, OH, ═O (where m is not 0) or a group capable of donating NO, or R⁴ may be H, SH, OH, substituted or unsubstituted alkyl, substituted or unsubstituted alkenyl, substituted or unsubstituted alkynyl, substituted or unsubstituted acyl, substituted or unsubstituted acyloxy, substituted or unsubstituted aminoalkyl, substituted or unsubstituted alkoxy, substituted or unsubstituted aryloxy, substituted or unsubstituted arylamine, substituted or unsubstituted arylsulphide, substituted or unsubstituted arylsulphone, substituted or unsubstituted arylsulfurdioxide, substituted or unsubstituted aryl, substituted or unsubstituted aryloxy, or substituted or unsubstituted heterocycle, wherein the optional substituent may be a group capable of donating NO;
R^{5A} and R^{5B} may be, independently, (CH₂)ₚ X³, p being from 0 to 4, and X³ being H, OH, ═O (where p is not 0) or a group capable of donating NO, or R^{5A} and R^{5B} may be, independently, H, OH, ═O, substituted or unsubstituted alkyl, substituted or unsubstituted alkenyl, substituted or unsubstituted alkynyl, substituted or unsubstituted acyl, substituted or unsubstituted acyloxy, substituted or unsubstituted aminoalkyl, substituted or unsubstituted alkoxy, substituted or unsubstituted arylamine, substituted or unsubstituted arylsulphide, substituted or unsubstituted arylsulphone, substituted or unsubstituted arylsulfurdioxide, substituted or unsubstituted aryl, substituted or unsubstituted aryloxy, or substituted or unsubstituted heterocycle, wherein the optional substituent may be a group capable of donating NO;
and wherein
X and Y may independently be —CH═CH—, (CH₂)_{q} while q is from 0 to 3, O, S, NH, CH₂, or NR⁷, wherein R⁷ may be hydrogen, substituted or unsubstituted alkyl, substituted or unsubstituted alkenyl, substituted or unsubstituted alkynyl, substituted or unsubstituted acyl, substituted or unsubstituted acyloxy, substituted or unsubstituted aminoalkyl, substituted or unsubstituted alkoxy, substituted or unsubstituted arylamine, substituted or unsubstituted arylsulphide, substituted or unsubstituted arylsulphone, substituted or unsubstituted arylsulfurdioxide, substituted or unsubstituted aryl, substituted or unsubstituted aryloxy, substituted or unsubstituted heterocycle, wherein the optional substituent may be a group capable of donating NO; and where ring B is a 5-, 6- or 7-membered ring.

67. A method according to claim 51, wherein said compound has Formula II 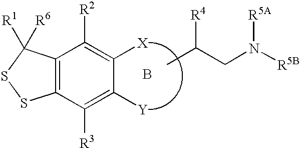
wherein R¹ and R⁶ may be independently hydrogen, substituted or unsubstituted alkyl, substituted or unsubstituted alkenyl, substituted or unsubstituted alkynyl, substituted or unsubstituted cycloalkyl, substituted or unsubstituted cycloalkenyl, substituted or unsubstituted alkoxy, substituted or unsubstituted aryloxy, substituted or unsubstituted aryl, or substituted or unsubstituted heterocycle, wherein the optional substituent may be a group capable of donating NO;
R² and R³ may be independently hydrogen, or (CH₂)ₙX¹ while n being from 0 to 4, and X¹ being H, OH, ═O (where n is not 0) or a group capable of donating NO, or R² and R³ may be independently H, OH, substituted or unsubstituted alkyl, substituted or unsubstituted alkenyl, substituted or unsubstituted alkynyl, substituted or unsubstituted cycloalkyl, substituted or unsubstituted cycloalkenyl, substituted or unsubstituted alkoxy, substituted or unsubstituted aryloxy, substituted or unsubstituted aryl, or substituted or unsubstituted heterocycles, wherein the optional substituent may be a group capable of donating NO;
R⁴ may be (CH₂)ₘX² while m being from 0 to 4, and X² being H, SH, OH, ═O (where m is not 0) or a group capable of donating NO, or R⁴ may be H, SH, OH, substituted or unsubstituted alkyl, substituted or unsubstituted alkenyl, substituted or unsubstituted alkynyl, substituted or unsubstituted acyl, substituted or unsubstituted acyloxy, substituted or unsubstituted aminoalkyl, substituted or unsubstituted alkoxy, substituted or unsubstituted arylamine, substituted or unsubstituted arylsulphide, substituted or unsubstituted arylsulphone, substituted or unsubstituted arylsulfurdioxide, substituted or unsubstituted aryl, substituted or unsubstituted aryloxy, or substituted or unsubstituted heterocycles, wherein the optional substituent may be a group capable of donating NO;
R^{5A} and R^{5B} may be, independently, (CH₂)ₚ X³ while p being from 0 to 4, and X³ being H, OH, ═O (where p is not 0) or a group capable of donating NO, or R^{5A} and R^{5B} may be, independently, H, OH, ═O, substituted or unsubstituted alkyl, substituted or unsubstituted alkenyl, substituted or unsubstituted alkynyl, substituted or unsubstituted acyl, substituted or unsubstituted acyloxy, substituted or unsubstituted aminoalkyl, substituted or unsubstituted alkoxy, substituted or unsubstituted arylamine, substituted or unsubstituted arylsulphide, substituted or unsubstituted arylsulphone, substituted or unsubstituted arylsulfurdioxide, substituted or unsubstituted aryl, substituted or unsubstituted aryloxy, or substituted or unsubstituted heterocycles, wherein the optional substituent may be a group capable of donating NO; and
X and Y may independently be —CH═CH—, (CH₂)_{q} while q being from 0 to 3, O, S, NH, CH₂, or NR⁷, wherein R⁷ may be hydrogen, substituted or unsubstituted alkyl, substituted or unsubstituted alkenyl, substituted or unsubstituted alkynyl, substituted or unsubstituted acyl, substituted or unsubstituted acyloxy, substituted or unsubstituted aminoalkyl, substituted or unsubstituted alkoxy, substituted or unsubstituted arylamine, substituted or unsubstituted arylsulphide, substituted or unsubstituted arylsulphone, substituted or unsubstituted arylsulfurdioxide, substituted or unsubstituted aryl, substituted or unsubstituted aryloxy, substituted or unsubstituted heterocycles; wherein the optional substituent may be a group capable of donating NO; and wherein ring B is a 5-, 6- or 7-membered ring.

68. A method according to claim 51, wherein said compound has Formula IA 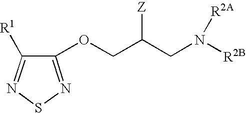
wherein R¹ is a group comprising a substituted N-oxide free radical, wherein the N-oxide free radical is contained within a 5- or 6-membered ring, and optionally further comprises a group capable of donating NO, or R¹ is a group selected from IIA, IIIA, IVA, and VA as shown below, where the groups of Formulae IIA-VA are linked to Formula IA at position R¹, through substituent Y of Formulae IIA-VA;
Z is halo, nitrato, nitroso, nitrile, hydroxyl, thiol, sulfonamido, amino, guanadino, isoguanadino, cyano, isocyano, and carboxyl; and
R^{2A} and R^{2B} are independently hydrogen, ═O, alkyl, substituted alkyl, alkenyl, substituted alkenyl, alkynyl, substituted alkynyl, cycloalkyl, substituted cycloalkyl, cycloalkenyl, substituted cycloalkenyl, phenyl, substituted phenyl or heterocyclyl, wherein the optional substitution comprises one or more groups capable of donating NO group, or one or more groups being ROS scavengers; and wherein
IIA has structure 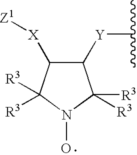
in which R³ is independently hydrogen or C₁-C₄ alkyl;
Y is selected from (CH₂)ₙ while n being from 0 to 3, O, NH, S, substituted or unsubstituted acyl, acyloxy, alkynyl, alkene, alkyl, alkoxy, aryloxy, arylamine, arylsulphide, arylsulphone, or arylsulfurdioxide; X is selected from (CHₘ)ₚ while m being 2 or 3 and p from 0 to 3, O, —N═N—, S, NH, CH₃N—, substituted or unsubstituted acyl, acyloxy, alkynyl, alkene, alkyl, alkoxy, aryloxy, arylamine, arylsulphide, arylsulphone, arylsulfurdioxide; and Z¹ is H OH, ONO, ONO₂, SNO;
IIIA has structure 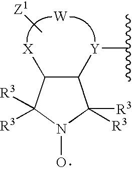
in which R³ is independently hydrogen or C₁-C₄ alkyl;
W is is (CHₘ)ₙ while m being from 0 to 2 and n being from 0 to 3, where W may be substituted or unsubstituted; X is selected from (CHₚ), while p 1 or 2, O, —N═N—, S, and NH, or X is absent; Y is (CH) or N; and Z¹ is selected from H, OH, CH₂ONO, CH₂CH₂ONO, CH₂ONO₂, CH₂CH₂ONO₂, ONO, ONO₂, SNO, and NONOate; wherein IIIA may include 1-3 Z¹ substituents on the ring to which Z¹ is attached;
IVA has structure 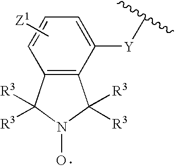
in which R³ may be independently hydrogen or C₁-C₄ alkyl; Y may be CH₂, O, NH, S, substituted or unsubstituted alkylene, or it can be absent; Z¹ may be CH₂ONO, CH₂CH₂ONO, CH₂ONO₂, CH₂CH₂ONO₂, NO, NO₂, ONO, ONO₂, SNO, or OH, wherein IVA may include 1-3 of the Z¹ substituents on the ring to which Z¹ is attached;
VA has structure 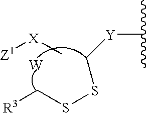
in which R³ is hydrogen or C₁-C₄ alkyl; W is (CHₘ)ₙ while m being from 0 to 2 and n being from 0 to 3, where W can be substituted or unsubstituted; X is selected from (CHₚ) while p being 1 or 2, O, —N═N—, S, NH, CH₃N—, and substituted or unsubstituted alkylene, or X is absent; Y is selected from CH₂, O, NH, S or substituted or unsubstituted alkylene, or Y is absent; and Z¹ is OH, ONO, ONO₂, N(NO)₂, or SNO.

69. A method according to claim 66, wherein R^{5A} and R^{5B} are selected as shown in Tables I′, II′, III′, and IV′
TABLE I′
i.
R^{5A}/R^{5B}
CH₃—
C₅H₉—
C₆H₅SO₂O—
CH₃CO—
C₆H₅SO₂NH—
(C₆H₅SO₂)₂N—
C₄H₈
C₅H₁₀
C₅H₁₁
TABLE II′

| R^{5A} | R^{5B} |
|---|---|
| CH₃— | —C(CH₃)₃ |
| (CH₃)₂CH— | —C(CH₃)₃ |
| CH₃CH₂CH₂— | —C(CH₃)₃ |
| CH₃— | —CH(CH₃)₂ |
| CH₃— | —C(CH₃)₂CH₂OH |
| CH₃CH₂CH₂CH₂— | —C(CH₃)₃ |
| CH₃— | 4-CF₃—Ph— |
| CH₃CH₂— | —C(CH₃)₃ |
| CH₃— | —CH₃ |
| CH₃— | 3,4,5-tri(CH₃O—)Ph— |

TABLE III′

| R^{5A} | R^{5B} |
|---|---|
| CH₃CH₂— | —C(CH₃)₃ |
| CH₃CH₂CH₂— | —C(CH₃)₃ |
| CH₃CH₂CH₂CH₂— | —C(CH₃)₃ |
| CH₃CH₂OC(O)CH₂CH₂— | —C(CH₃)₃ |
| CH₃CH₂OC(O)CH₂— | —C(CH₃)₃ |

TABLE IV′

| R^{5A} | R^{5B} |
|---|---|
| CH₃— | —C(CH₃)₃ |
| CH₃—O—CH₂CH₂— | —C(CH₃)₃ |
| CH₃— | —CH₂CH₂CH(SCH₃)CH₃ |

70. A method according to claim 67, wherein R^{5A} and R^{5B} are selected as shown in Tables I′, II′, III′, and IV′
TABLE I
ii.
R^{5A}/R^{5B}
CH₃—
C₅H₉—
C₆H₅SO₂O—
CH₃CO—
C₆H₅SO₂NH—
(C₆H₅SO₂)₂N—
C₄H₈
C₅H₁₀
C₅H₁₁
TABLE II

| R^{5A} | R^{5B} |
|---|---|
| CH₃— | —C(CH₃)₃ |
| (CH₃)₂CH— | —C(CH₃)₃ |
| CH₃CH₂CH₂— | —C(CH₃)₃ |
| CH₃— | —CH(CH₃)₂ |
| CH₃— | —C(CH₃)₂CH₂OH |
| CH₃CH₂CH₂CH₂— | —C(CH₃)₃ |
| CH₃— | 4-CF₃—Ph— |
| CH₃CH₂— | —C(CH₃)₃ |
| CH₃— | —CH₃ |
| CH₃— | 3,4,5-tri(CH₃O—)Ph— |

TABLE III

| R^{5A} | R^{5B} |
|---|---|
| CH₃CH₂— | —C(CH₃)₃ |
| CH₃CH₂CH₂— | —C(CH₃)₃ |
| CH₃CH₂CH₂CH₂— | —C(CH₃)₃ |
| CH₃CH₂OC(O)CH₂CH₂— | —C(CH₃)₃ |
| CH₃CH₂OC(O)CH₂— | —C(CH₃)₃ |

TABLE IV

| R^{5A} | R^{5B} |
|---|---|
| CH₃— | —C(CH₃)₃ |
| CH₃—O—CH₂CH₂— | —C(CH₃)₃ |
| CH₃— | —CH₂CH₂CH(SCH₃)CH₃ |

71. A method according to claim 68, wherein R^{2A} and R^{2B} are selected as shown in Tables I, II, III, and IV
TABLE I
iii.
R^{2A}/R^{2B}
CH₃—
C₅H₉—
C₆H₅SO₂O—
CH₃CO—
C₆H₅SO₂NH—
(C₆H₅SO₂)₂N—
C₄H₈
C₅H₁₀
C₅H₁₁
TABLE II

| R^{2A} | R^{2B} |
|---|---|
| CH₃— | —C(CH₃)₃ |
| (CH₃)₂CH— | —C(CH₃)₃ |
| CH₃CH₂CH₂— | —C(CH₃)₃ |
| CH₃— | —CH(CH₃)₂ |
| CH₃— | —C(CH₃)₂CH₂OH |
| CH₃CH₂CH₂CH₂— | —C(CH₃)₃ |
| CH₃— | 4-CF₃—Ph— |
| CH₃CH₂— | —C(CH₃)₃ |
| CH₃— | —CH₃ |
| CH₃— | 3,4,5-tri(CH₃O—)Ph— |

TABLE III

| R^{2A} | R^{2B} |
|---|---|
| CH₃CH₂— | —C(CH₃)₃ |
| CH₃CH₂CH₂— | —C(CH₃)₃ |
| CH₃CH₂CH₂CH₂— | —C(CH₃)₃ |
| CH₃CH₂OC(O)CH₂CH₂— | —C(CH₃)₃ |
| CH₃CH₂OC(O)CH₂— | —C(CH₃)₃ |

TABLE IV

| R^{2A} | R^{2B} |
|---|---|
| CH₃— | —C(CH₃)₃ |
| CH₃—O—CH₂CH₂— | —C(CH₃)₃ |
| CH₃— | —CH₂CH₂CH(SCH₃)CH₃ |

72. A method according to claim 51, wherein said multifunctional β-blocker compound has formula III 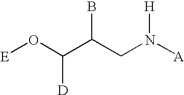
wherein A is C₁-C₄ alkyl or ROS-scavenger group;
B is selected from OH, O—NO₂ and SH;
D is H, or D is (CH₂)₂ and is connected to E and together with the neighboring atoms forms a 5-6 membered ring consisting of carbon atoms and one oxygen atom; and
E is phenyl condensed with optionally substituted phenyl or optionally substituted 5-6 membered heterocycle containing one of —N—, —O—, and —S—S—; or
E is thiadiazolyl substituted with morpholinyl or pyrrolidinyl-N-oxide, said morpholinyl being optionally substituted with one of OH, NO-donor group, and ROS-scavenger group, and said pyrrolidinyl-N-oxide group being bound to said thiadiazolyl vial —S— or via —CH₂—O—.

73. A method according to claim 51, wherein said multifunctional β-blocker compound is selected from the group consisting of compounds nos. 14, 15, 20-75, 1′, 2′, and 7′-24′ as shown below:

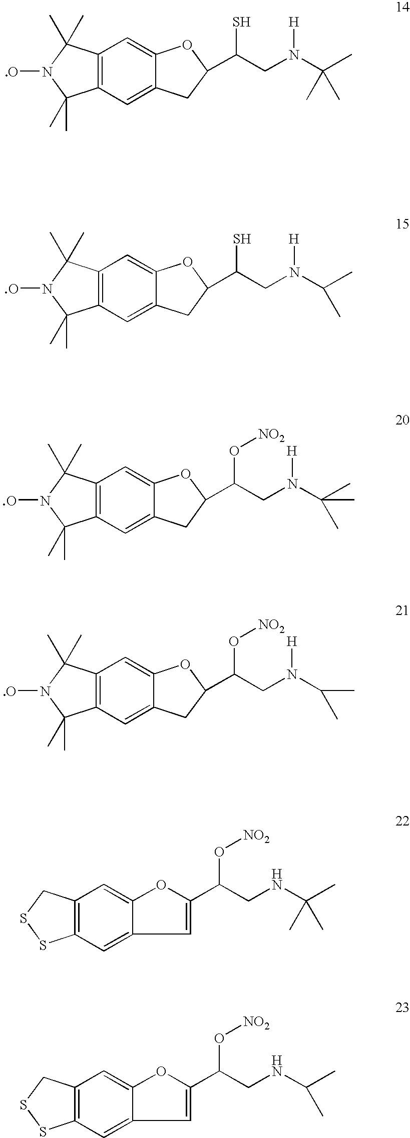

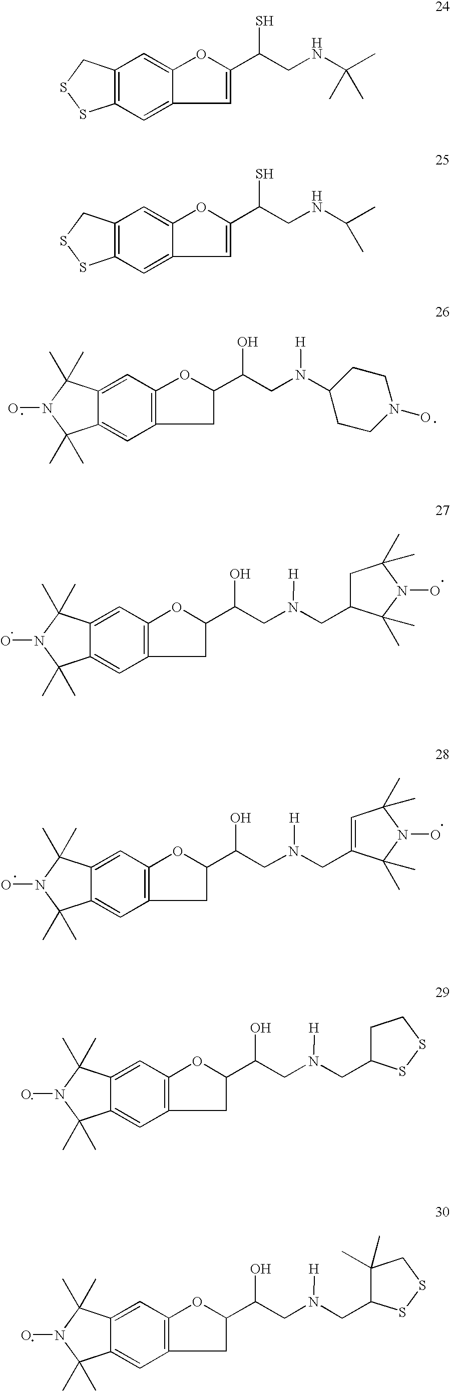

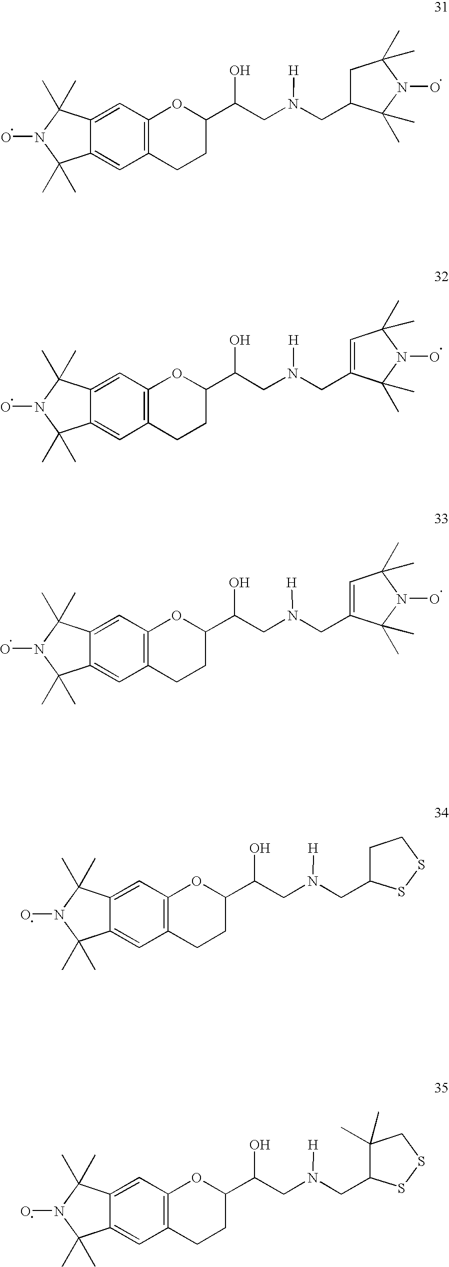

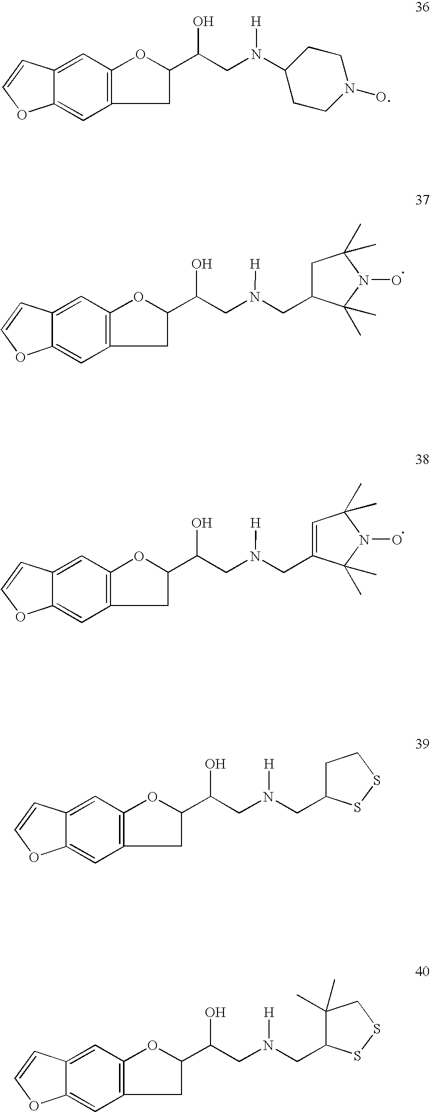

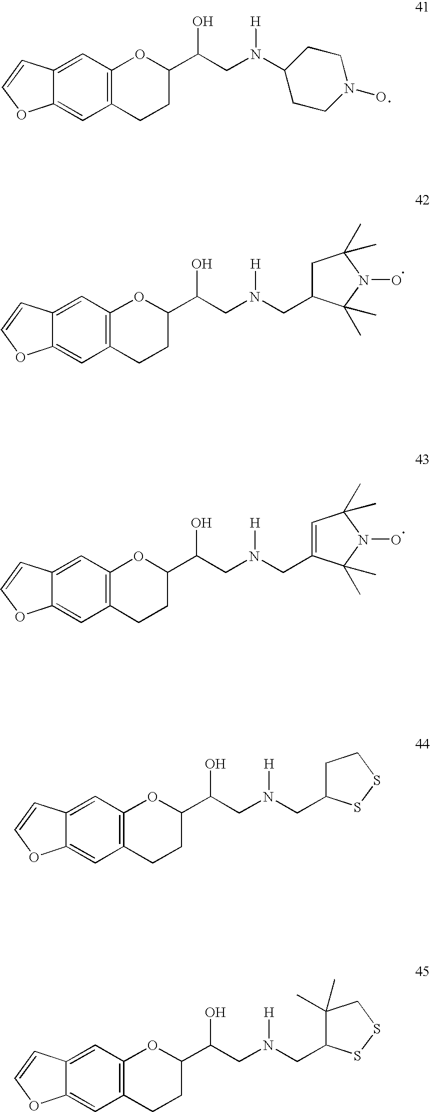

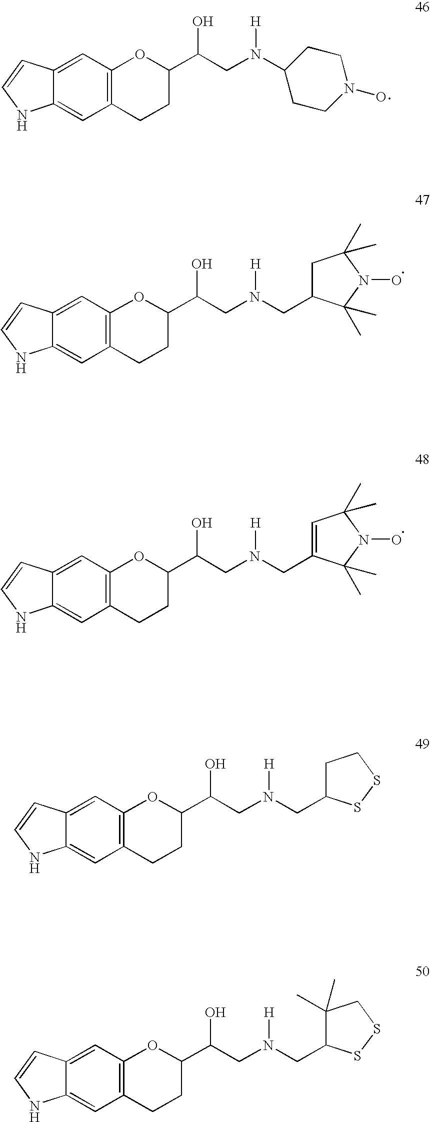

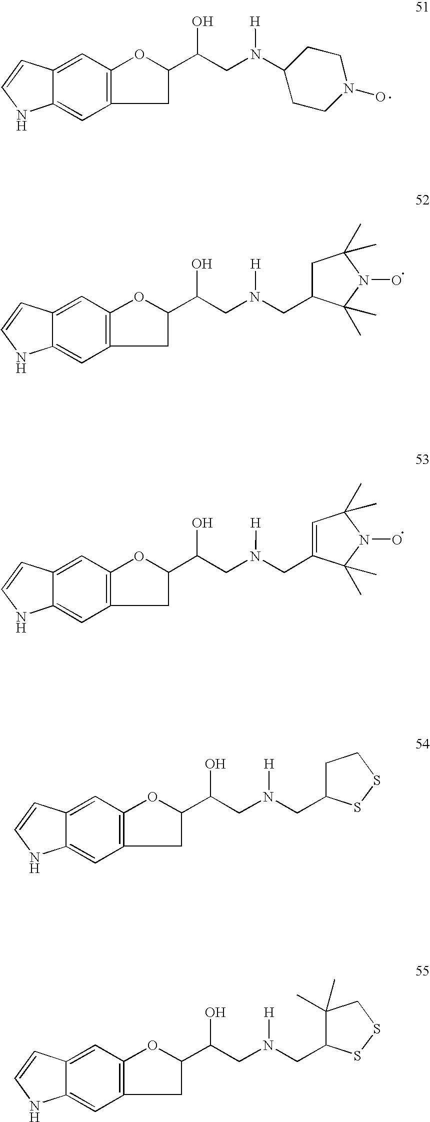

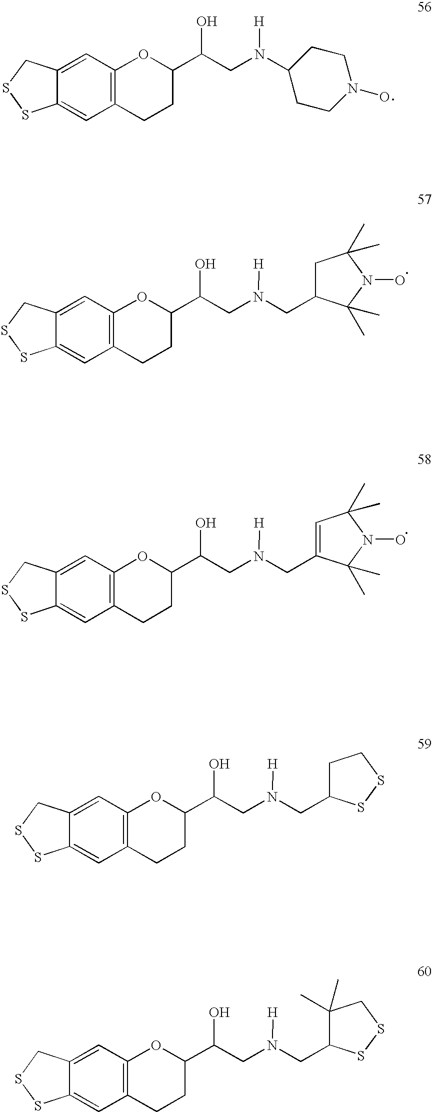

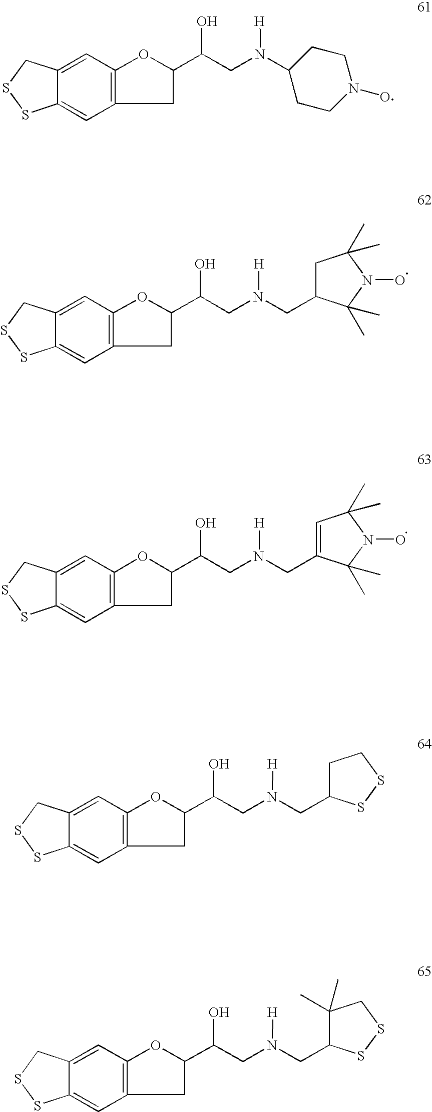

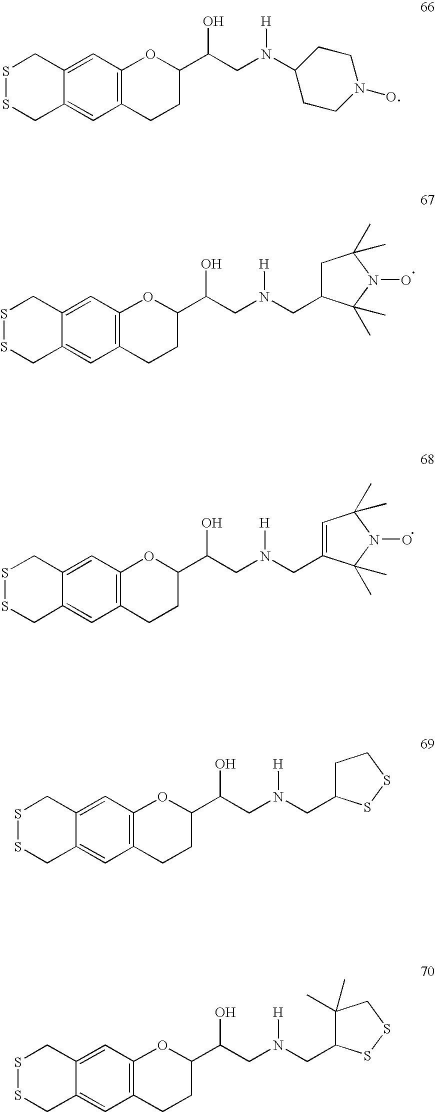

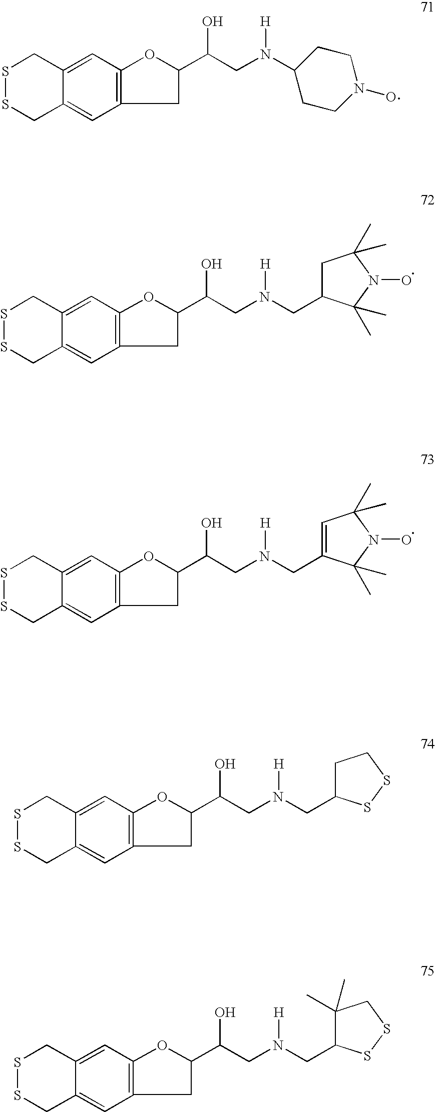

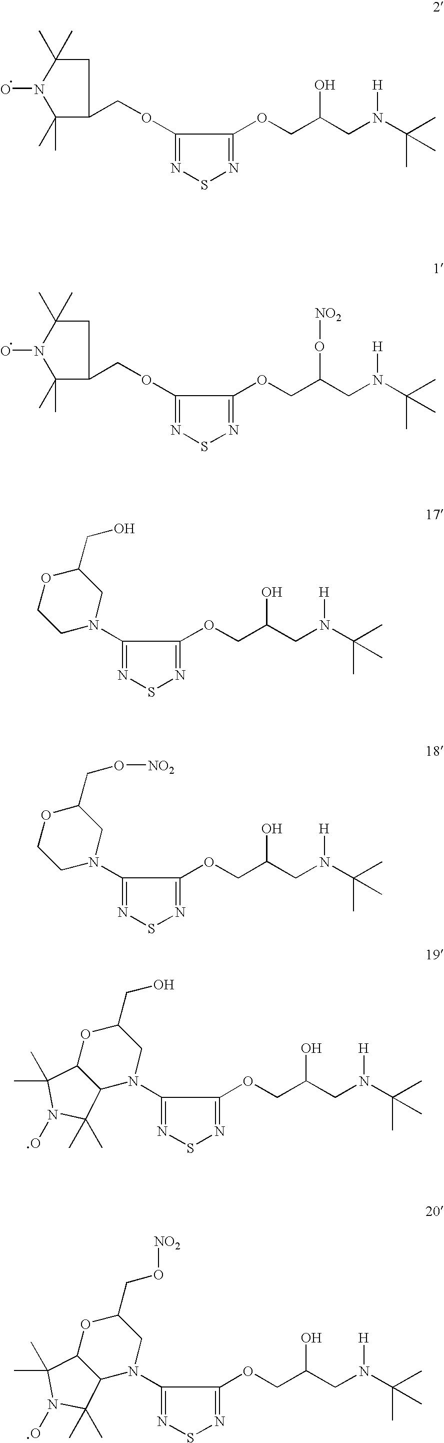

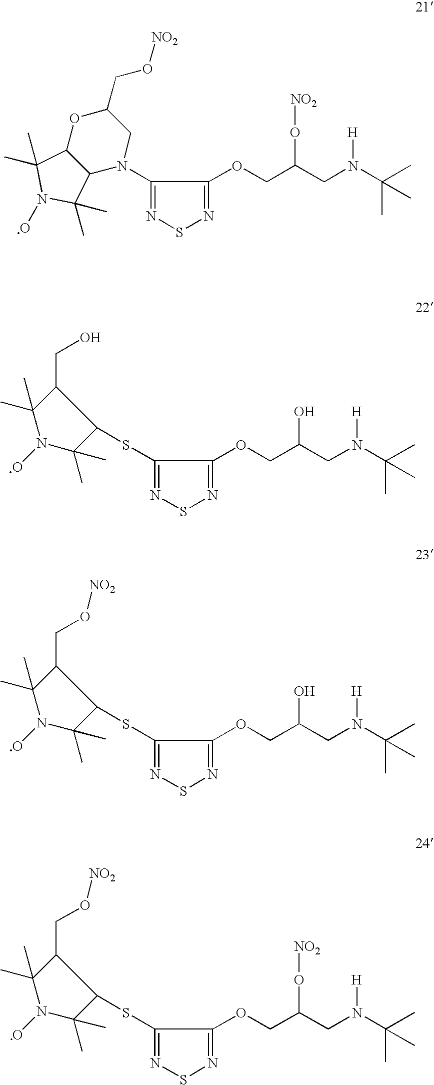

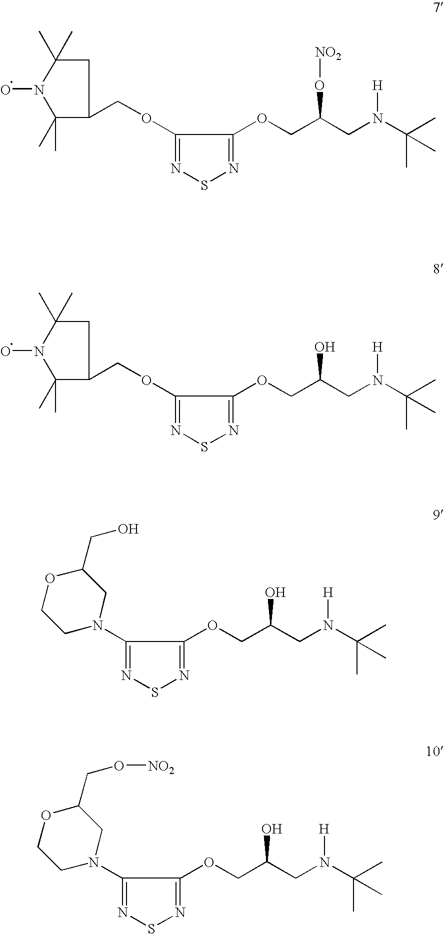

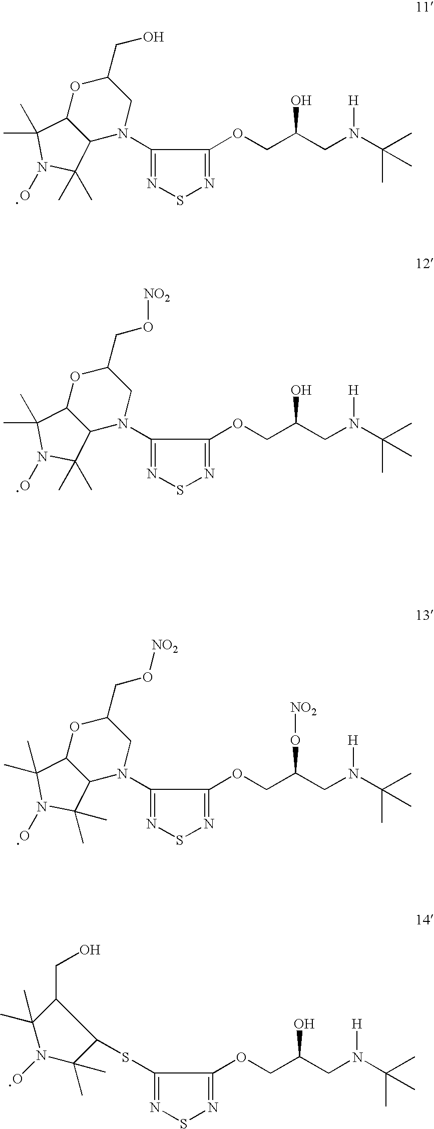

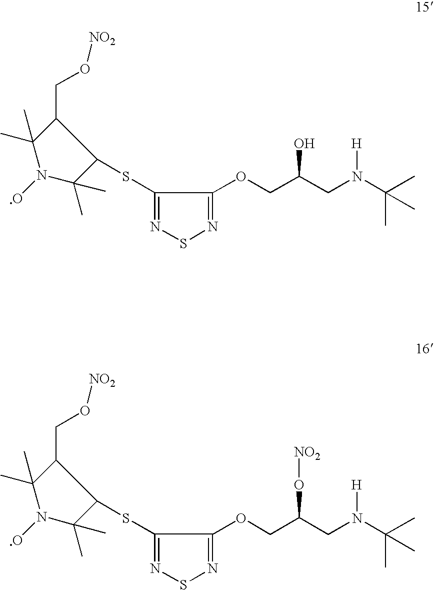

74. A method according to claim 51, wherein said administration or treatment is selected from the group consisting of topical, oral, and parenteral.

75. A method according to claim 51, wherein said administration or treatment is selected from the group consisting of suppository, by way of injection, and by way of infusion.

76. A method according to claim 51, wherein said multifunctional blocker compound is administered by a route selected from intramuscular, intraperitoneal, intravenous, ICV, intracisternal injection or infusion, subcutaneous injection, implant, inhalation spray, nasal, vaginal, rectal, sublingual, and urethral.

77. A method according to claim 1, wherein said mammal is human.

78. A multifunctional β-adrenergic receptor antagonist compound comprising
i) a β-blocker component,
ii) at least one ROS-scavenger component, and
iii) at least one NO-donor component.

79. A multifunctional antagonist according to claim 78, wherein said ROS-scavenger component comprises N-oxide free radical, wherein the nitrogen of said N-oxide free radical is within a 3-, 4-, 5-, 6- or 7-membered ring, wherein the ring may be substituted or unsubstituted with straight or branched alkyl groups, alkoxy groups or groups capable of donating NO.

80. A multifunctional antagonist according to claim 78, wherein said compound has Formula I 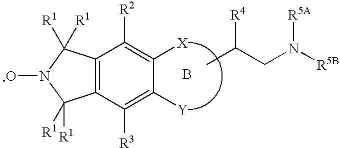
wherein R¹ may be independently hydrogen, substituted or unsubstituted alkyl, substituted or unsubstituted alkenyl, substituted or unsubstituted alkynyl, substituted or unsubstituted cycloalkyl, substituted or unsubstituted cycloalkenyl, substituted or unsubstituted alkoxy, substituted or unsubstituted aryloxy, substituted or unsubstituted aryl, or substituted or unsubstituted heterocycles, wherein the optional substituent may be a group capable of donating NO;
R² and R³ may be independently hydrogen or (CH₂)ₙX¹, n being from 0 to 4, and X¹ being H, OH, ═O (where n is not 0) or a group capable of donating NO, or
R² and R³ may be independently selected from H, OH, substituted or unsubstituted alkyl, substituted or unsubstituted alkenyl, substituted or unsubstituted alkynyl, substituted or unsubstituted cycloalkyl, substituted or unsubstituted cycloalkenyl, substituted or unsubstituted aryl, and substituted or unsubstituted heterocycle, wherein the optional substituent may be a group capable of donating NO;
R⁴ may be (CH₂)ₘX², m being from 0 to 4, and X² being H, SH, OH, ═O (where m is not 0) or a group capable of donating NO, or R⁴ may be H, SH, OH, substituted or unsubstituted alkyl, substituted or unsubstituted alkenyl, substituted or unsubstituted alkynyl, substituted or unsubstituted acyl, substituted or unsubstituted acyloxy, substituted or unsubstituted aminoalkyl, substituted or unsubstituted alkoxy, substituted or unsubstituted aryloxy, substituted or unsubstituted arylamine, substituted or unsubstituted arylsulphide, substituted or unsubstituted arylsulphone, substituted or unsubstituted arylsulfurdioxide, substituted or unsubstituted aryl, substituted or unsubstituted aryloxy, or substituted or unsubstituted heterocycle, wherein the optional substituent may be a group capable of donating NO;
R^{5A} and R^{5B} may be, independently, (CH₂)ₚ X³, p being from 0 to 4, and X³ being H, OH, ═O (where p is not 0) or a group capable of donating NO, or R^{5A} and R^{5B} may be, independently, H, OH, ═O, substituted or unsubstituted alkyl, substituted or unsubstituted alkenyl, substituted or unsubstituted alkynyl, substituted or unsubstituted acyl, substituted or unsubstituted acyloxy, substituted or unsubstituted aminoalkyl, substituted or unsubstituted alkoxy, substituted or unsubstituted arylamine, substituted or unsubstituted arylsulphide, substituted or unsubstituted arylsulphone, substituted or unsubstituted arylsulfurdioxide, substituted or unsubstituted aryl, substituted or unsubstituted aryloxy, or substituted or unsubstituted heterocycle, wherein the optional substituent may be a group capable of donating NO;
and wherein
X and Y may independently be —CH═CH—, (CH₂)_{q} while q is from 0 to 3, O, S, NH, CH₂, or NR⁷, wherein R⁷ may be hydrogen, substituted or unsubstituted alkyl, substituted or unsubstituted alkenyl, substituted or unsubstituted alkynyl, substituted or unsubstituted acyl, substituted or unsubstituted acyloxy, substituted or unsubstituted aminoalkyl, substituted or unsubstituted alkoxy, substituted or unsubstituted arylamine, substituted or unsubstituted arylsulphide, substituted or unsubstituted arylsulphone, substituted or unsubstituted arylsulfurdioxide, substituted or unsubstituted aryl, substituted or unsubstituted aryloxy, substituted or unsubstituted heterocycle, wherein the optional substituent may be a group capable of donating NO, and where ring B is a 5-, 6- or 7-membered ring.

81. A multifunctional antagonist according to claim 78, wherein said compound has Formula II 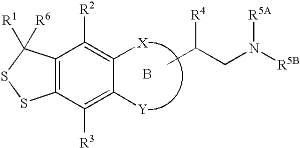
wherein R¹ and R⁶ may be independently hydrogen, substituted or unsubstituted alkyl, substituted or unsubstituted alkenyl, substituted or unsubstituted alkynyl, substituted or unsubstituted cycloalkyl, substituted or unsubstituted cycloalkenyl, substituted or unsubstituted alkoxy, substituted or unsubstituted aryloxy, substituted or unsubstituted aryl, or substituted or unsubstituted heterocycle, wherein the optional substituent may be a group capable of donating NO;
R² and R³ may be independently hydrogen, or (CH₂)ₙX¹ while n being from 0 to 4, and X¹ being H, OH, ═O (where n is not 0) or a group capable of donating NO, or R² and R³ may be independently H, OH, substituted or unsubstituted alkyl, substituted or unsubstituted alkenyl, substituted or unsubstituted alkynyl, substituted or unsubstituted cycloalkyl, substituted or unsubstituted cycloalkenyl, substituted or unsubstituted alkoxy, substituted or unsubstituted aryloxy, substituted or unsubstituted aryl, or substituted or unsubstituted heterocycles, wherein the optional substituent may be a group capable of donating NO;
R⁴ may be (CH₂)ₘX² while m being from 0 to 4, and X² being H, SH, OH, ═O (where m is not 0) or a group capable of donating NO, or R⁴ may be H, SH, OH, substituted or unsubstituted alkyl, substituted or unsubstituted alkenyl, substituted or unsubstituted alkynyl, substituted or unsubstituted acyl, substituted or unsubstituted acyloxy, substituted or unsubstituted aminoalkyl, substituted or unsubstituted alkoxy, substituted or unsubstituted arylamine, substituted or unsubstituted arylsulphide, substituted or unsubstituted arylsulphone, substituted or unsubstituted arylsulfurdioxide, substituted or unsubstituted aryl, substituted or unsubstituted aryloxy, or substituted or unsubstituted heterocycles, wherein the optional substituent may be a group capable of donating NO;
R^{5A} and R^{5B} may be, independently, (CH₂)ₚ X³ while p being from 0 to 4, and X³ being H, OH, ═O (where p is not 0) or a group capable of donating NO, or R^{5A} and R^{5B} may be, independently, H, OH, ═O, substituted or unsubstituted alkyl, substituted or unsubstituted alkenyl, substituted or unsubstituted alkynyl, substituted or unsubstituted acyl, substituted or unsubstituted acyloxy, substituted or unsubstituted aminoalkyl, substituted or unsubstituted alkoxy, substituted or unsubstituted arylamine, substituted or unsubstituted arylsulphide, substituted or unsubstituted arylsulphone, substituted or unsubstituted arylsulfurdioxide, substituted or unsubstituted aryl, substituted or unsubstituted aryloxy, or substituted or unsubstituted heterocycles, wherein the optional substituent may be a group capable of donating NO; and
X and Y may independently be —CH═CH—, (CH₂)_{q} while q being from 0 to 3, O, S, NH, CH₂, or NR⁷, wherein R⁷ may be hydrogen, substituted or unsubstituted alkyl, substituted or unsubstituted alkenyl, substituted or unsubstituted alkynyl, substituted or unsubstituted acyl, substituted or unsubstituted acyloxy, substituted or unsubstituted aminoalkyl, substituted or unsubstituted alkoxy, substituted or unsubstituted arylamine, substituted or unsubstituted arylsulphide, substituted or unsubstituted arylsulphone, substituted or unsubstituted arylsulfurdioxide, substituted or unsubstituted aryl, substituted or unsubstituted aryloxy, substituted or unsubstituted heterocycles; wherein the optional substituent may be a group capable of donating NO; and wherein ring B is a 5-, 6- or 7-membered ring.

82. A multifunctional antagonist according to claim 78, wherein said compound has Formula IA 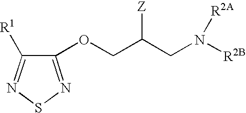
wherein R¹ is a group comprising a substituted N-oxide free radical, wherein the N-oxide free radical is contained within a 5- or 6-membered ring, and optionally further comprises a group capable of donating NO, or R¹ is a group selected from IIA, IIIA, IVA, and VA as shown below, where the groups of Formulae IIA-VA are linked to Formula IA at position R¹, through substituent Y of Formulae IIA-VA;
Z is halo, nitrato, nitroso, nitrile, hydroxyl, thiol, sulfonamido, amino, guanadino, isoguanadino, cyano, isocyano, and carboxyl; and
R^{2A} and R^{2B} are independently hydrogen, ═O, alkyl, substituted alkyl, alkenyl, substituted alkenyl, alkynyl, substituted alkynyl, cycloalkyl, substituted cycloalkyl, cycloalkenyl, substituted cycloalkenyl, phenyl, substituted phenyl or heterocyclyl, wherein the optional substitution comprises one or more groups capable of donating NO group, or one or more groups being ROS scavengers; and wherein
IIA has structure 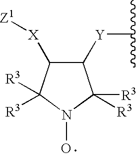
in which R³ is independently hydrogen or C₁-C₄ alkyl;
Y is selected from (CH₂)ₙ while n being from 0 to 3, O, NH, S, substituted or unsubstituted acyl, acyloxy, alkynyl, alkene, alkyl, alkoxy, aryloxy, arylamine, arylsulphide, arylsulphone, or arylsulfurdioxide; X is selected from (CHₘ)ₚ while m being 2 or 3 and p from 0 to 3, O, —N═N—, S, NH, CH₃N—, substituted or unsubstituted acyl, acyloxy, alkynyl, alkene, alkyl, alkoxy, aryloxy, arylamine, arylsulphide, arylsulphone, arylsulfurdioxide; and Z¹ is H OH, ONO, ONO₂, SNO;
IIIA has structure 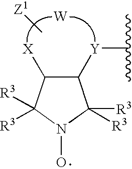
in which R³ is independently hydrogen or C₁-C₄ alkyl;
W is is (CHₘ)ₙ while m being from 0 to 2 and n being from 0 to 3, where W may be substituted or unsubstituted; X is selected from (CHₚ) while p 1 or 2, O, —N═N—, S, and NH, or X is absent; Y is (CH) or N; and Z¹ is selected from H, OH, CH₂ONO, CH₂CH₂ONO, CH₂ONO₂, CH₂CH₂ONO₂, ONO, ONO₂, SNO, and NONOate; wherein IIIA may include 1-3 Z¹ substituents on the ring to which Z¹ is attached;
IVA has structure 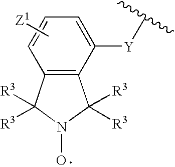
in which R³ may be independently hydrogen or C₁-C₄ alkyl; Y may be CH₂, O, NH, S, substituted or unsubstituted alkylene, or it can be absent; Z¹ may be CH₂ONO, CH₂CH₂ONO, CH₂ONO₂, CH₂CH₂ONO₂, NO, NO₂, ONO, ONO₂, SNO, or OH, wherein IVA may include 1-3 of the Z¹ substituents on the ring to which Z¹ is attached;
VA has structure 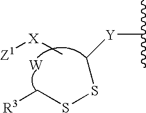
in which R³ is hydrogen or C₁-C₄ alkyl; W is (CHₘ)ₙ while m being from 0 to 2 and n being from 0 to 3, where W can be substituted or unsubstituted; X is selected from (CHₚ) while p being 1 or 2, O, —N═N—, S, NH, CH₃N—, and substituted or unsubstituted alkylene, or X is absent; Y is selected from CH₂, O, NH, S or substituted or unsubstituted alkylene, or Y is absent; and Z¹ is OH, ONO, ONO₂, N(NO)₂, or SNO.

83. A mulrifunctional agonist according to claim 78, wherein said compound is selected from the group consisting of compounds nos. 14, 15, 20-75, 1′, 2′, and 7′-24′ as shown below:

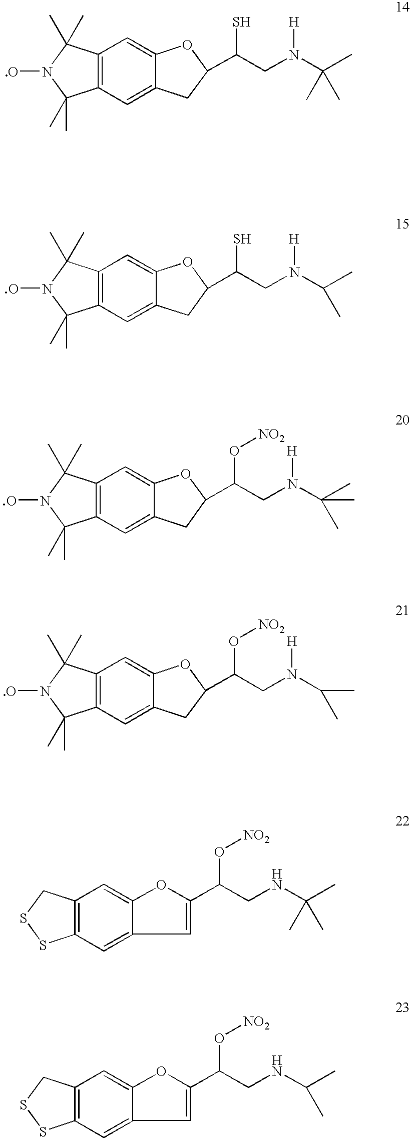

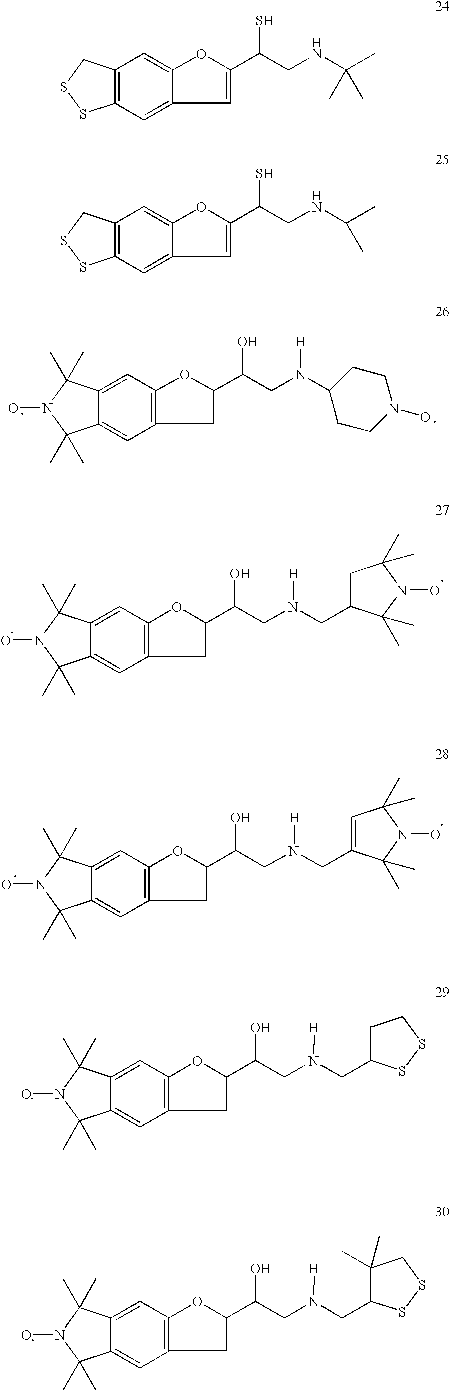

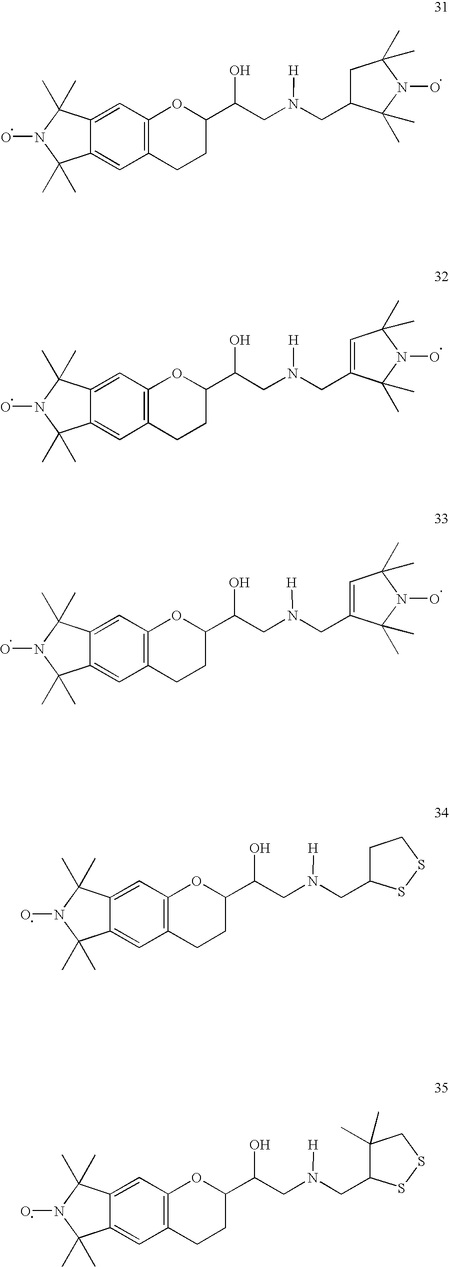

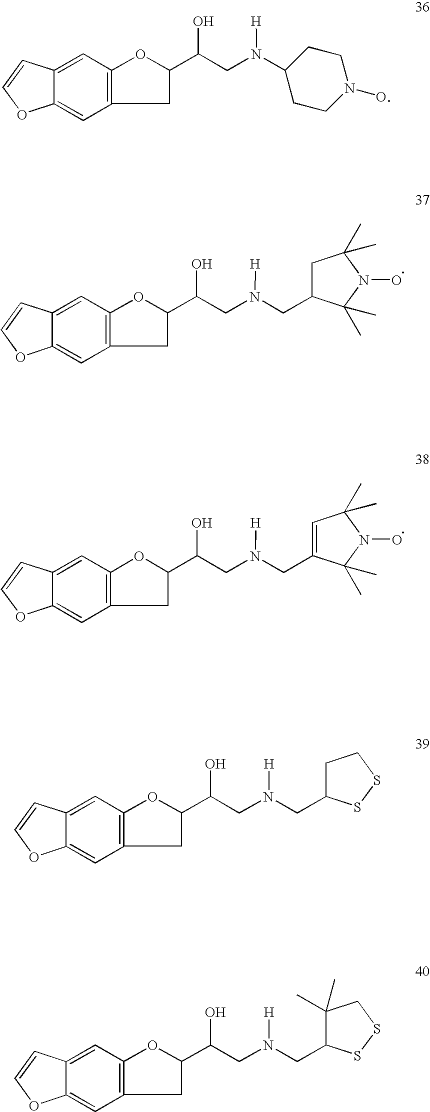

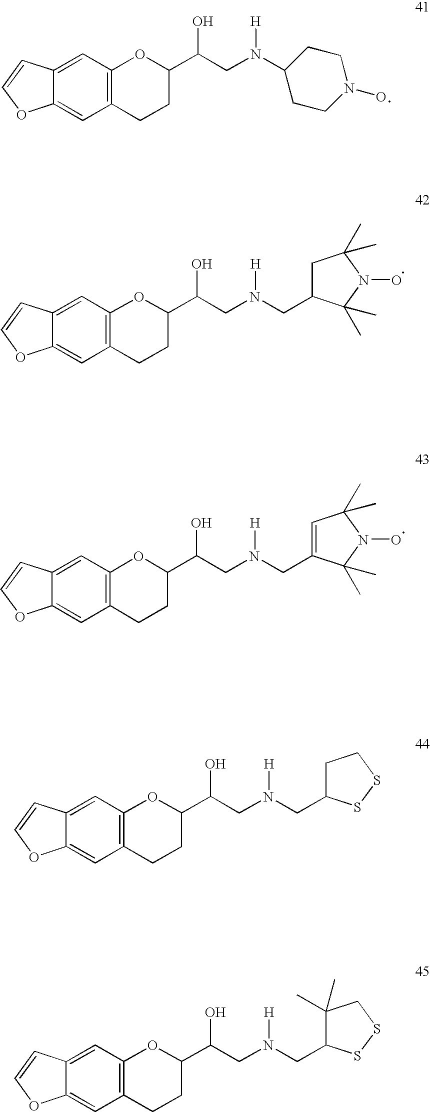

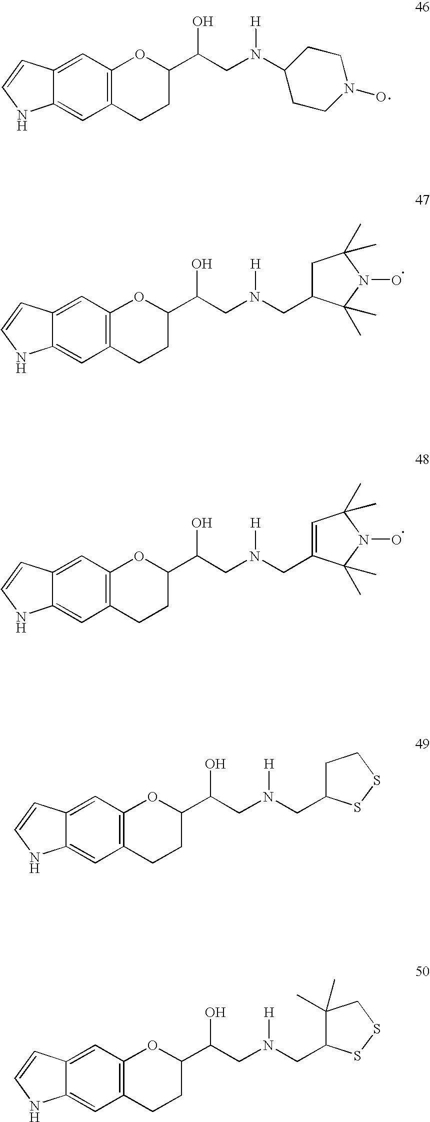

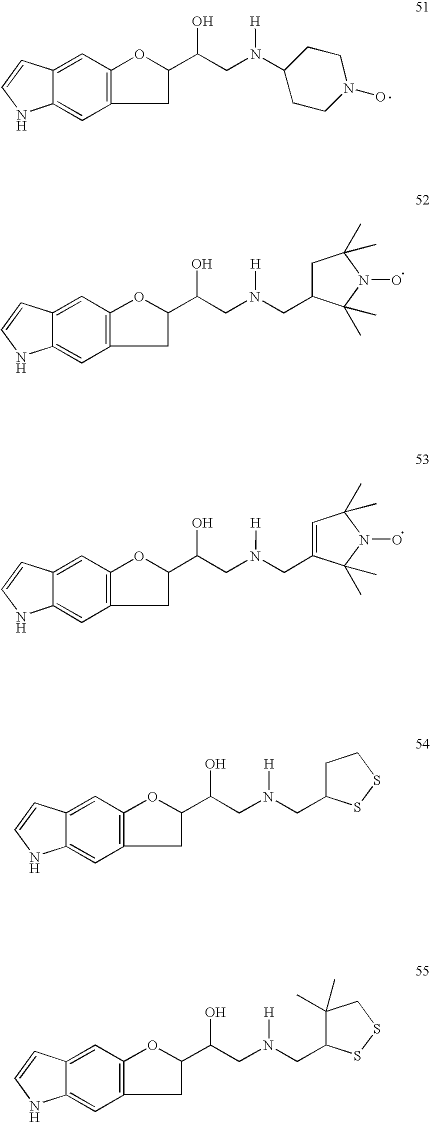

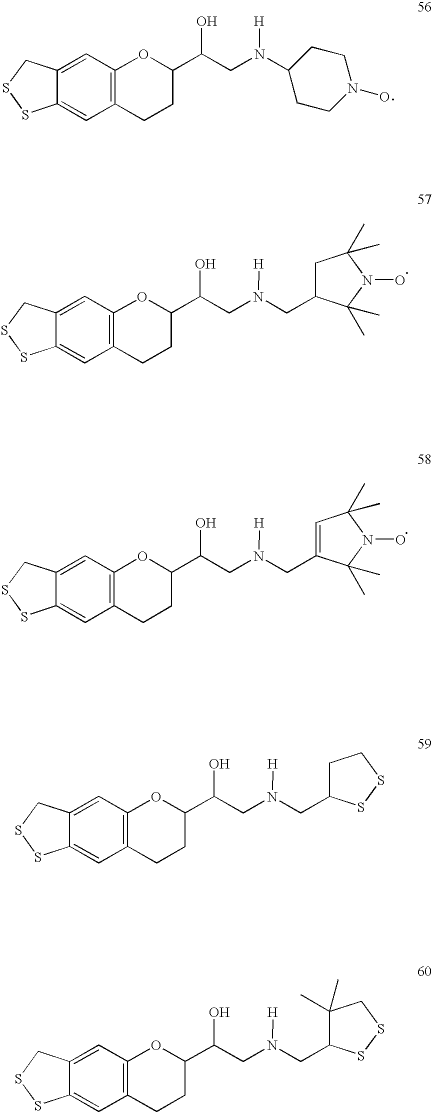

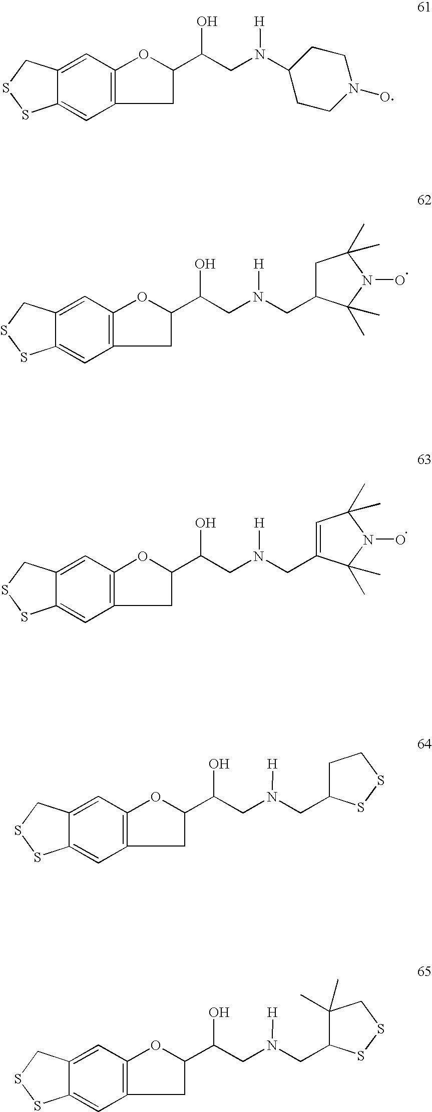

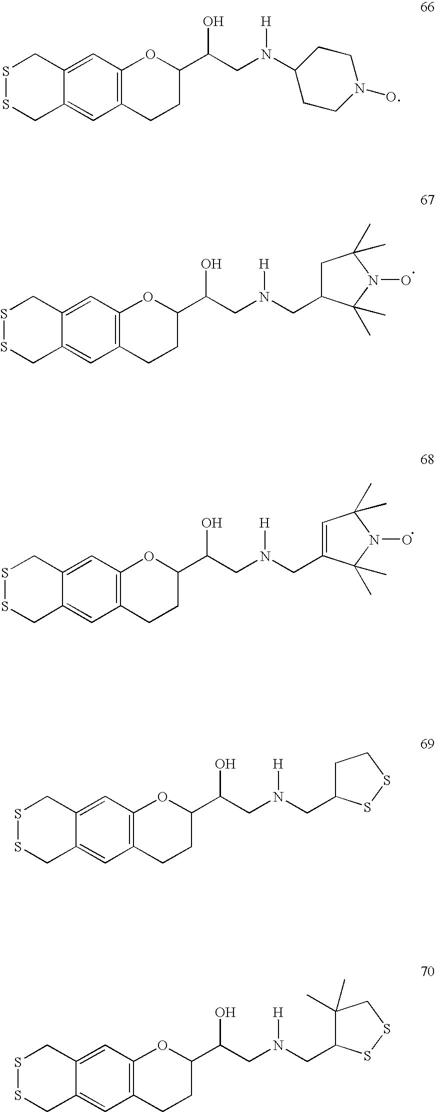

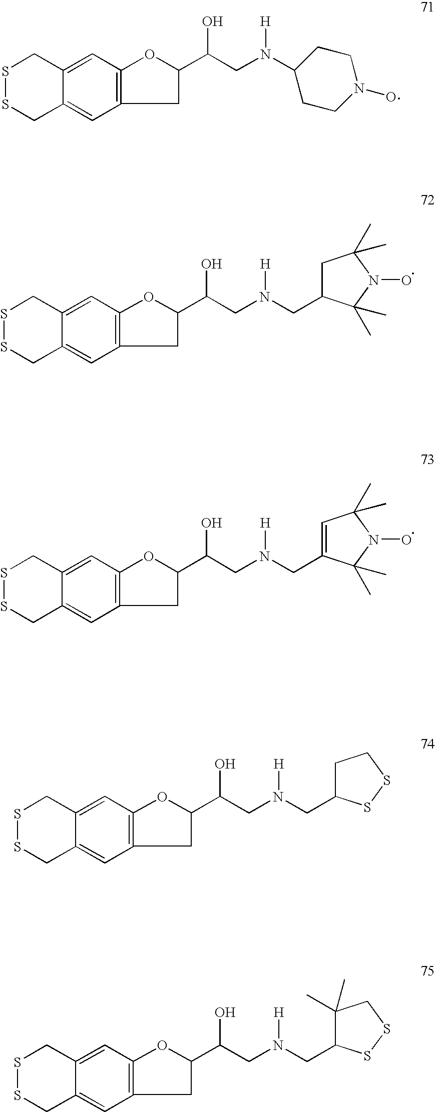

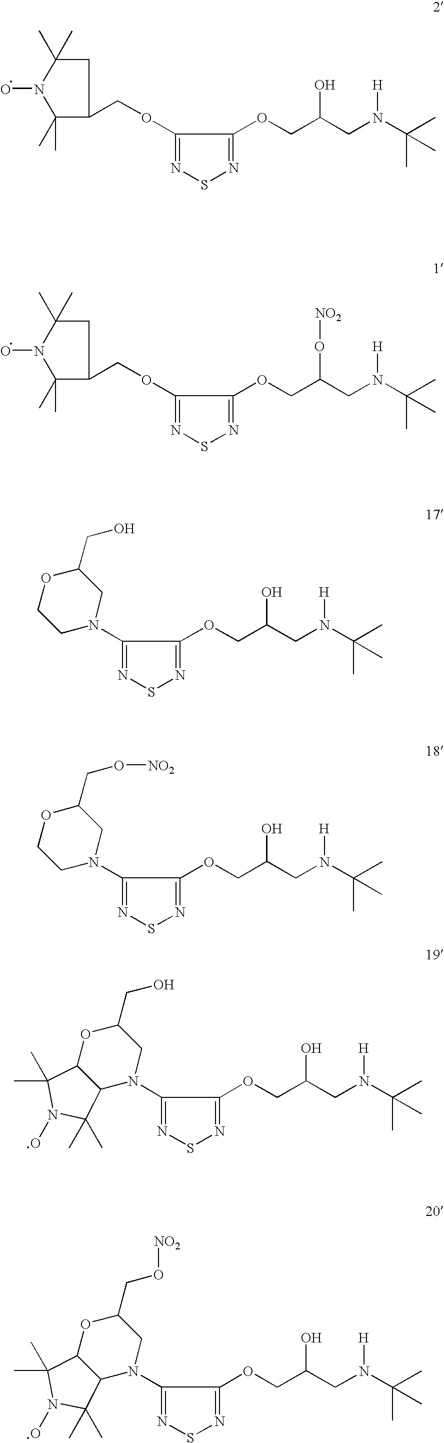

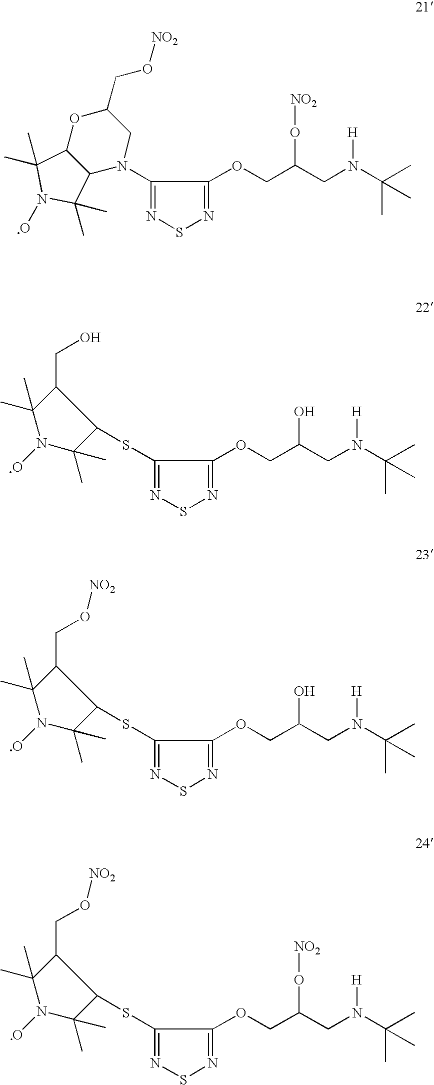

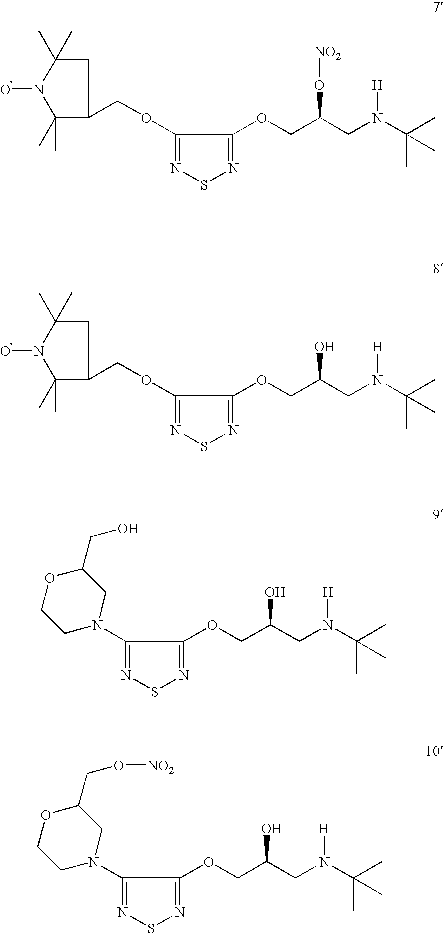

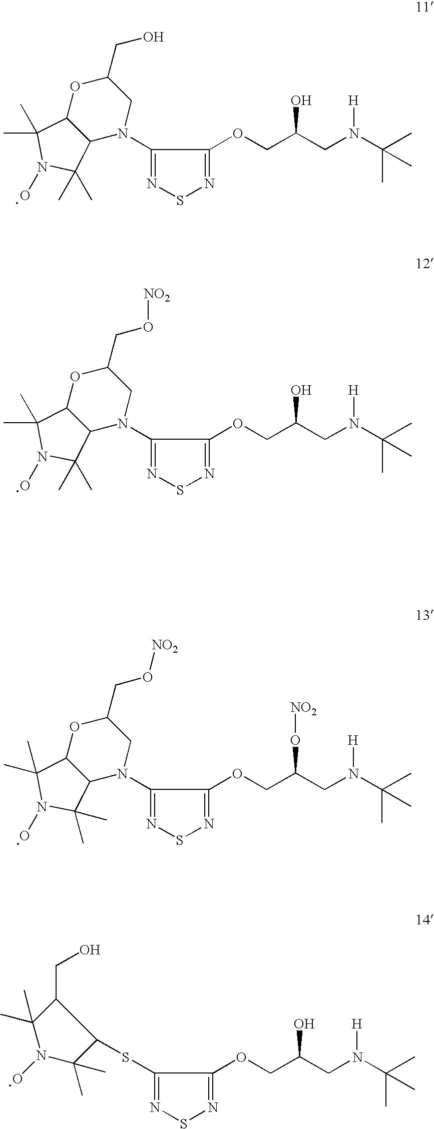

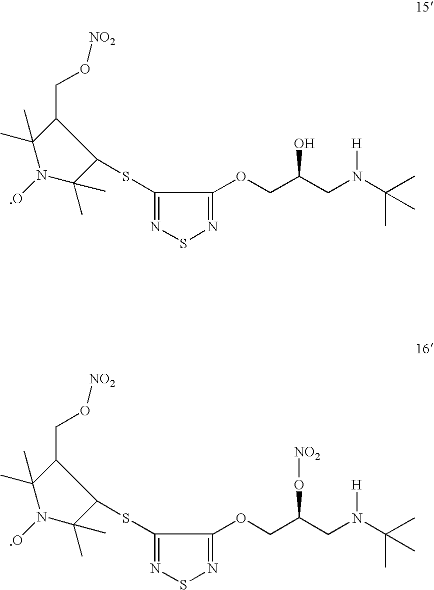

84. A pharmaceutical composition comprising a compound according to claim 78, or a solvate, optical isomer, or salt thereof.

85. A pharmaceutical composition according to claim 84, further comprising a component selected from a carrier, binding agent, stabilizer, adjuvant, diluent, excipient, surfactant, odorant, and a second pharmaceutically active agent.

86. A kit for administering a multifunctional β-adrenergic receptor antagonist compound comprising
i) a dosage amount of at least one compound having β-blocker activity and ROS-scavenging activity,
ii) instructions for use; and
iii) optionally, means for the delivery of said compound.
